(19) United States
(12) Patent Application Publication
(10) Pub. No.: US 2026/0083724 A1
(43) Pub. Date: Mar. 26, 2026

(54) COMPOUNDS TARGETING PAX3::FOXO1 FUSION PROTEIN

(71) Applicant: Georgetown University, Washington, DC (US)

(72) Inventors: Aykut Üren, Rockville, MD (US); Jeffrey A. Toretsky, Silver Spring, MD (US)

(73) Assignee: Georgetown University, Washington, DC (US)

(21) Appl. No.: 19/341,825

(22) Filed: Sep. 26, 2025

Related U.S. Application Data
(60) Provisional application No. 63/699,556, filed on Sep. 26, 2024.

(51) Int. Cl. A61K 31/4709; A61P 35/00

(52) U.S. Cl. CPC A61K 31/4709; A61P 35/00

(57) ABSTRACT

Compounds comprising a PAX3::FOXO1 fusion protein binding moiety, a bivalent linker, and an E3 ubiquitin ligase binding moiety. The compounds can be used to reduce PAX3::FOXO1 protein levels in a subject with fusion-positive rhabdomyosarcoma (FP-RMS), to ubiquitinate a PAX3::FOXO1 fusion protein in a cell, to treat FP-RMS in a subject in need thereof, and/or to treat a disease caused by the overexpression of PAX3::FOXO1 protein in a subject in need thereof.

### CROSS-REFERENCE TO RELATED APPLICATIONS

This application claims benefit of U.S. Provisional Application No. 63/699,556, filed on Sep. 26, 2024, which is incorporated herein by reference in its entirety.

### SEQUENCE LISTING

The instant application contains a Sequence Listing, which has been submitted electronically in XML format and is hereby incorporated by reference in its entirety. Said XML copy, created on Sep. 26, 2025, is named Georgtown_050_US1-Sequence_Listing, and is 4,670 bytes in size.

### BACKGROUND OF THE INVENTION

Rhabdomyosarcoma (RMS) is a cancer of skeletal muscle tissue and is the most common soft-tissue sarcoma in children and adolescents, responsible for 3% of all childhood malignant tumors [1]. There are approximately 350 new cases of RMS each year [2].

RMS tumors can be categorized based on whether they harbor a chromosomal translocation that encode for a fusion protein (fusion-positive) or do not harbor such a chromosomal location (fusion-negative). The implication of the difference is significant, as patients with fusion-positive RMS (FP-RMS) tumors have a lower survival rate than those with fusion-negative RMS (FN-RMS) tumors [3, 4].

The two most common subtypes of RMS are embryonal rhabdomyosarcoma (ERMS) and alveolar rhabdomyosarcoma (ARMS). ARMS is associated with a lower survival rate [5], with approximately 80% of ARMS cases being fusion-positive, and is characterized by tumor-specific chromosomal translocations [t (2; 13) or t (1; 13)] that generates a PAX3::FOXO1 or a PAX7::FOXO1 gene fusion [6-8]. PAX3::FOXO1 is the more common fusion protein, occurring in approximately 70% of fusion-positive RMS (FP-RMS) [9]. The PAX3::FOXO1 and PAX7::FOXO1 fusion proteins retain the DNA binding domains of the PAX3 and PAX7 proteins and the transcriptional activation domain of FOXO1, and are ten to 100-fold more transcriptionally active at PAX3/7 DNA binding sites than either wild-type PAX3 or PAX7 [6, 7, 10-12].

The PAX3::FOXO1 fusion protein in particular has been studied for its role in tumorigenesis. For example, expression of PAX3::FOXO1 in a regulatable myoblast model was found to cause colony formation in vitro and tumor formation in vivo, while reduced expression of this fusion protein inhibited both phenotypes [13]. Further, PAX3::FOXO1 reduction in a xenograft model sensitized tumors to radiation [14]. These studies demonstrate that the PAX3::FOXO1 fusion protein is a key regulator of fusion-positive RMS oncogenesis.

Consequently, direct inhibitors of PAX3::FOXO1 have been developed and studied for their potential use as a therapy. Several drugs that inhibit PAX3::FOXO1 responsive reporter constructs or phenocopy the loss of the PAX3::FOXO1 gene expression profile have been reported, including thapsigargin, entinostat, fenretinide, JQ1, SAHA, and tideglusib [15-20]. Other molecules such as rapamycin and ponatinib tested in RMS models target PAX3::FOXO1-activated signaling cascades [21, 22]. However, none of these drugs directly bind to PAX3::FOXO1 to specifically inhibit its function.

Therefore, there is a need in the art for direct inhibitors of the PAX3::FOXO1 fusion protein that can be used in patients suffering from ARMS.

### SUMMARY OF INVENTION

The present invention relate to proteolysis-targeting chimeras (PROTAC) compounds that target the PAX3::FOXO1 fusion protein, their use, and methods of manufacturing the compounds.

In one aspect, the present invention provides compounds of formula (I):

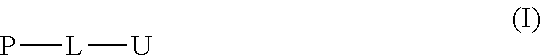

or a pharmaceutically acceptable salt thereof, in which:
P is a PAX3::FOXO1 fusion protein binding moiety;
L is a bivalent linker; and
U is an E3 ubiquitin ligase binding moiety.

The PAX3::FOXO1 fusion protein binding moiety comprises an agent selected from Table 1 or a derivative thereof, or any pharmaceutically salt of an agent selected from Table 1 or a derivative thereof. In some embodiments, the PAX3::FOXO1 fusion protein binding moiety comprises an agent selected from NSC697468, alcuronium chloride, NSC2805, didanosine, carboplatin, and piperacetazine, or a derivative thereof. In certain embodiments, the PAX3::FOXO1 fusion protein binding moiety comprises a derivative of piperacetazine. In particular embodiments, the derivative of piperacetazine is selected from C6.12, C6.16, C6.21, C6.14, C6.15, C6.18a, C6.18b, and any pharmaceutically salt thereof. In certain embodiments, the PAX3::FOXO1 fusion protein binding moiety comprises NSC697468, C6.12, C6.15, or C6.18b.

In some embodiments, the E3 ubiquitin ligase binding moiety comprises a ligand that binds to Von Hippel-Lindau tumor suppressor protein (VHL). In certain embodiments, the E3 ubiquitin ligase binding moiety comprises a ligand selected from VH032, VH101, VH298, and derivatives thereof.

In some embodiments, the E3 ubiquitin ligase binding moiety comprises a ligand that binds to cereblon (CRBN). In certain embodiments, the E3 ubiquitin ligase binding moiety comprises a ligand selected from thalidomide, pomalidomide, lenalidomide, 4-fluoro-thalidomide, and derivatives thereof.

In some embodiments, the E3 ubiquitin ligase binding moiety comprises a ligand that binds to cellular inhibitor of apoptosis protein (cIAP). In certain embodiments, the E3 ubiquitin ligase binding moiety comprises a ligand selected from methylbestatin, derivatives of methylbestatin, and LCL161 derivative.

In some embodiments, the E3 ubiquitin ligase binding moiety comprises a ligand that binds to murine double minute 2 (MDM2). In certain embodiments, the E3 ubiquitin ligase binding moiety comprises a ligand selected from nutlin, idasanutlin, and derivatives thereof.

In some embodiments, the E3 ubiquitin ligase binding moiety comprises a ligand that binds to S-phase kinase-associated protein 1 (SKP-1). In certain embodiments, the E3 ubiquitin ligase binding moiety comprises EN884 or a derivative thereof. In particular embodiments, the E3 ubiquitin ligase binding moiety comprises a derivative of EN884 selected from AD-5-49, AD-5-47a, and AD-5-47b.

In some embodiments, the bivalent linker comprises one or more alkyl chains, polyethylene glycol (PEG) chains, extended glycol chains, or a combination thereof. In certain embodiments, the bivalent linker comprises about 3 to 50 atoms in length.

In another aspect, the present invention provides a pharmaceutical composition comprising a compound of formula (I) of the present invention, and a pharmaceutically acceptable carrier.

In another aspect, the present invention provides a method of reducing PAX3::FOXO1 protein levels in a subject with FP-RMS, in which the method comprises administering the pharmaceutical composition of the present invention to the subject. In some embodiments, the subject has FP-alveolar RMS (FP-ARMS).

In a further aspect, the present invention provides a method of ubiquitinating a PAX3::FOXO1 fusion protein in a cell, in which the method comprises administering the pharmaceutical composition of present invention to the cell. In some embodiments, the cell is a FP-RMS tumor cell. In certain embodiments, the cell is a FP-ARMS tumor cell.

In another aspect, the present invention provides a method of treating FP-RMS in a subject in need thereof, in which the method comprises administering the pharmaceutical composition of the present invention to the subject. In some embodiments, the FP-RMS is FP-ARMS.

In yet another aspect, the present invention provides a method of treating a disease caused by the overexpression of PAX3::FOXO1 protein in a subject in need thereof, in which the method comprises administering the pharmaceutical composition of the present invention to the subject. In certain embodiments, the disease is biphenotypic sinonasal sarcoma.

### BRIEF DESCRIPTION OF THE FIGURES

FIG. 1 shows results demonstrating purification of recombinant PAX3::FOXO1 protein from bacteria, as described in Example 1. Panel A shows PAX3::FOXO1 protein expressed in Escherichia coli (E. coli). The most prominent bands following induction with isopropyl-beta-D-thiogalactopyranoside (IPTG) appeared just above the 100 kDa molecular weight marker on an 8% polyacrylamide gel stained with Coomassie blue. F17-F23 represent seven different fractions purified by column chromatography using the His tag on the carboxy terminal of the protein. Panel B shows that the same purified fractions in Panel A were used in a Western blot analysis to confirm protein identity using an anti-PAX3 antibody. Panel C shows DNA-binding specificity evaluated by measuring binding of recombinant PAX3::FOXO1 and EWS::FLI1 proteins to double-stranded DNA oligonucleotides with binding sequences specific to cither protein. PAX3::FOXO1 or EWS::FLI1 protein (a tumor-specific fusion oncoprotein found in Ewing sarcoma) was immobilized on a CM5 chip and the indicated oligonucleotides were injected over the surface at a 1.25 to 80 nmol/L dose range. Panel D shows the quality of the PAX3::FOXO1 protein evaluated by measuring its binding affinity to a double-stranded DNA oligonucleotide. PAX3::FOXO1 was immobilized on a CM5 chip and the oligonucleotide was injected over the surface at six concentrations (62.5, 12.5, 2.5, 0.5, 0.1, 0.02 nmol/L). Binding affinity was calculated by using a 1:1 binding model in BiaEvaluation software (χ2:0.0424, U-value: 2). Lighter lines indicate actual datapoints and black lines indicate curve fit used for affinity analysis.

FIG. 2 shows results of screening small-molecule libraries for PAX3::FOXO1 binding, as described in Example 1. Panel A shows binding of individual compounds from Prestwick or DTP libraries, presented as fold difference of PAX3::FOXO1 binding compared with the negative control protein (CD99). Each dot represents an individual compound. The horizontal line indicates the 5-fold binding threshold used for the selection of primary hits. Piperacetazine is labeled with a star. Panel B shows inhibition of luciferase activity in fusion-positive RMS cell line RH30 with PAX3::FOXO1-responsive 6XPRS reporter and renal cell carcinoma cell line SN12C with phosphoglycerate kinase (PGK) reporter (negative control). Responses are normalized to dimethyl sulfoxide (DMSO) control. Compounds in the top left quadrant are considered potential PAX3::FOXO1 inhibitors, piperacetazine is labeled with a star. Panel C shows PAX3::FOXO1-responsive platelet-derived growth factor receptor alpha (PDGFRA) luciferase reporter transfected into RH30 and RD cell lines and treated with the top six hits identified by the secondary screening as shown in Panel B. The cells were lysed for luciferase assay 48 hours posttreatment, and the luciferase signal was quantified. (****, P<0.0001; ***, P<0.001; **, P<0.01; *, P<0.05; Student t test; ns, P>0.05). Panel D shows the structure of piperacetazine. Panel E shows direct binding of piperacetazine to PAX3::FOXO1 protein analyzed by surface plasmon resonance (SPR). Piperacetazine was injected at 1.25, 2.5, 5, and 10 μmol/L concentrations in triplicates. A representative experiment is shown. An average equilibrium dissociation constant (K_{D}) of 3.8 μmol/L was calculated from six experiments (Inset). Panel F shows RH30 cells lysates incubated with 30 μmol/L piperacetazine or DMSO and heated to the indicated temperatures. Lysates were centrifuged to separate precipitated proteins and soluble protein levels were measured by Western blot analysis using FOXO1 antibody (in each blot, top band: PAX3::FOXO1, bottom band: FOXO1). Panel G shows quantification of the three replicate experiments provided in a line graph by plotting mean and SE values.

FIG. 3 shows results demonstrating that piperacetazine inhibits endogenous PAX3::FOXO1 activity in multiple cell lines, as described in Example 1. Panel A shows the effect of piperacetazine treatment on cell viability measured using the CellTiter-Blue assay (lines with square marks). The effect of piperacetazine treatment on PAX3::FOXO1 activity was measured via luciferase assay (lines with circular marks), using the PAX3::FOXO1-responsive pGL3-PDGFRA luciferase reporter (RH30, RH41, RH28, U66788, RD), and PGK reporter (SN12C, negative control). Panel B shows, on the left, luciferase activity measured in HEK293T cells transfected with PAX3::FOXO1-responsive ASS1P reporter and an empty or PAX3::FOXO1 expression vector. Cells were treated with DMSO or piperacetazine for 48 hours. Matching Western blots are shown under each bar. Panel B also shows, on the right, the same experimental setup repeated with EWS::FLI1-responsive NR0B1 luciferase reporter and an EWS::FLI1 expression vector or empty vector. The cells were treated with the same concentration of piperacetazine or DMSO for 48 hours, and the normalized luciferase readouts and accompanying Western blots are shown. (*, P<0.0001; Student t test; ns, P>0.05)

FIG. 4 shows results demonstrating that piperacetazine inhibits PAX7::FOXO1 activity, as described in Example 1. A luciferase assay was performed to measure the effect of piperacetazine on PAX7::FOXO1 activity. HEK293T cells were co-transfected with a pGL4.19-ASS1P luciferase reporter and a PAX7::FOXO1 pcDNA3 construct and treated for 48 hours with DMSO or 10 μM piperacetazine. At the end of the experiment, the cells were counted and lysed for the luciferase assay and for BCA protein quantification for normalization. The remaining cells were collected and lysed for western blot analysis. (****, p<0.0001, Student's t test).

FIG. 5 shows the chemical processes for synthesizing PROTAC Compounds A (Panel A), B (Panel B), C (Panel C), D (Panel D), and G (Panel E), as described in Example 2.

FIG. 6 shows results demonstrating cell survival of RH30 and TC71 cells exposed to PROTAC Compounds A (Panel A), B (Panel B), C (Panel C), D (Panel D), and G (Panel E) at a concentration ranging from 0.045 to 100 μM or 48 hours, as described in Example 2. The horizonal line indicates the half maximal inhibitory concentration (IC₅₀).

FIG. 7 shows Western blot analysis results of PAX3::FOXO1 degradation at eight and 24 in RH30 cells treated with PROTAC Compounds A, B, C, D, and G, as described in Example 2. The RH30 cells were grown to 60 to 80% confluence and treated either with the PROTAC compounds at a concentration of 10 mM (Panel A), or with PROTAC Compound B at concentrations of 30, 10, and 2 μM (Panel B). Beta-actin was used as a loading control.

FIG. 8 shows analysis results of PAX3::FOXO1 degradation in RH30 cells treated with PROTAC Compound 01-A1, as described in Example 3. Western blot images are shown for RH30 cells treated with Compound 01-A1 at a concentration of 3 μM (Panel A); RH30 cells treated with Compound 01-A1 at a concentration of 10 μM and with MG132 at a concentration of 1 μM (Panel B); and RH30 cells treated with 50 μg/ml of CHX or treated with 50 μg/ml of CHX and Compound 01-A1 at a concentration of 10 μM (Panel C). Panel D shows proteins levels of the RH30 cells treated with 50 μg/ml of CHX or treated with 50 μg/ml of CHX and Compound 01-A1 at a concentration of 10 μM.

FIG. 9 shows analysis results of PAX3::FOXO1 degradation in RH30 cells or RH41 cells treated with PROTAC Compound 01-E1, as described in Example 3. Western blot images are shown for RH30 cells treated with Compound 01-E1 at a concentration of 3 μM (Panel A); RH30 cells treated with Compound 01-E1 at a concentration of 500 nM, 1, μM, 3 μM, and 10 μM (Panel B); RH30 cells treated with Compound 01-E1 or treated with C6.18 (Panel C); and RH30 cells treated with Compound 01-E1 at a concentration of 10 μM and with MG132 at a concentration of 1 μM (Panel D).

FIG. 10 shows analysis results of PAX3::FOXO1 degradation in RH30 cells treated with PROTAC Compound 07-A, as described in Example 4. Western blot images are shown for RH30 cells treated with Compound 07-A at a concentration of 3 μM (Panel A); RH30 cells treated with Compound 07-A at a concentration of 10 μM (Panel B); and RH30 cells treated with 50 μg/ml of CHX or treated with 50 μg/ml of CHX and Compound 07-A at a concentration of 10 μM (Panel D). Panel C shows proteins levels of the RH30 cells treated with Compound 07-A versus DMSO as a control, and Panel E shows proteins levels of the RH30 cells treated with 50 μg/ml of CHX or treated with 50 μg/ml of CHX and Compound 07-A at a concentration of 10 μM.

FIG. 11 shows analysis results of PAX3::FOXO1 degradation in RH30 cells treated with PROTAC Compound 07-E, as described in Example 4. Western blot images are shown for RH30 cells treated with Compound 07-E at a concentration of 3 μM (Panel A); RH30 cells treated with Compound 07-E at a concentration of 10 μM (Panel B); RH30 cells treated with Compound 07-E at a concentration of 10 μM and with MG132 at a concentration of 1 μM or 10 μM (Panel D); and RH30 cells treated with 50 μg/ml of CHX or treated with 50 μg/ml of CHX and Compound 07-E at a concentration of 10 μM (Panel E). Panel C shows proteins levels of the RH30 cells treated with Compound 07-E versus DMSO as a control, and Panel F shows proteins levels of the RH30 cells treated with 50 μg/ml of CHX or treated with 50 μg/ml of CHX and Compound 07-E at a concentration of 10 μM.

FIG. 12 shows results of analyzing binding of Compounds G-3A, G-3B, and G-3C to PAX3::FOXO1 protein, as described in Example 5. The analysis was performed using SPR. Binding sensorgram are shown for G-3A (Panel A), G-3B (Panel B), and G-3C (Panel C).

FIG. 13 shows analysis results of PAX3::FOXO1 degradation in RH30 cells treated with PROTAC Compounds G-3A, G-3B, and G-3C, as described in Example 5. Western blot images are shown for RH30 cells treated with G-3A (Panel A), G-3B (Panel B), and G-3C (Panel C) at concentrations ranging from 100 nM to either 30 μM (for Compounds G-3A and G-3C) or to 3 μM (for Compound G-3B). Panel D shows proteins levels of the RH30 cells treated with each of the PROTAC compounds.

FIG. 14 shows analysis results of PAX3::FOXO1 degradation in RH30 cells treated with PROTAC Compounds G-3A, G-3B, and G-3C, and with MG132 at a concentration of 1 μM, as described in Example 5. Panel A shows Western blot image for RH30 cells treated with Compound G-3A at concentrations of 500 nM, 1 μM, and 3 μM; with Compound G-3B at a concentration of 1 μM; and with Compound G-3C at a concentration of 3 μM. Panel B shows proteins levels of the treated RH30 cells.

### DETAILED DESCRIPTION

The practice of the present invention can employ, unless otherwise indicated, conventional techniques of protein engineering, organic chemistry, biochemistry, protein chemistry, cell biology, oncology, and clinical practice, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a”, “an”, and “the” include plural referents, unless the context clearly dictates otherwise. The terms “a” (or “an”) as well as the terms “one or more” and “at least one” can be used interchangeably.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments.

As used herein, the term “or” is to be interpreted as an inclusive “or” meaning any one or any combination. Therefore, “A, B, or C” means any of the following: A; B; C; A and B; A and C; B and C; A, B, and C. An exception to this definition will occur only when a combination of elements, functions, steps or acts are in some way inherently mutually exclusive.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value (i.e., intermediate) encompassed by the range, including integers and fractions. For example, a stated range of 5-10 is also a disclosure of 5, 6, 7, 8, 9, and 10 individually, and of 5.2, 7.5, 8.7, and so forth.

Unless otherwise indicated, the terms “at least” or “about” preceding a series of elements is to be understood to refer to every element in the series. The term “about” preceding a numerical value includes ±10% of the recited value. For example, a concentration of about 1 mg/ml includes 0.9 mg/ml to 1.1 mg/ml. Likewise, a concentration range of about 1% to 10% (w/v) includes 0.9% (w/v) to 11% (w/v).

A “subject” or “individual” or “patient” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc. In preferred embodiments, a subject refers to a human.

An “ubiquitin ligase binding moiety” as used herein refers to a moiety (e.g., a small molecule) that binds to a ubiquitin ligase. In certain embodiments, the ubiquitin ligase binding moiety is an E3 ubiquitin ligase binding moiety.

The term “pharmaceutical composition” refers to a preparation that is in such form as to permit the biological activity of the active ingredient to be effective, and which contains no additional components that are unacceptably toxic to a subject to which the composition would be administered. Such composition can be sterile and can comprise a pharmaceutically acceptable carrier.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. In certain embodiments, a subject is successfully “treated” for a disease or disorder if the subject shows total, partial, or transient alleviation or elimination of at least one symptom or measurable physical parameter associated with the disease or disorder.

Terms such as “inhibit or “inhibiting” or “block” or “blocking” or “suppress” or “suppressing” or “reduce” or “reducing” are used interchangeably and refer to any statistically significant decrease in a given biological activity, including full blocking of the activity. For example, “inhibition” can refer to a decrease of about 10%, 20%, 30%, 40%, 50%, 60%, 70%, 80%, 90% or 100% in biological activity.

An “effective amount” as used herein refers to that amount of a compound or pharmaceutical composition which, when administered, is sufficient to carry out a specifically stated purpose. In the context of a method of treatment for a disease or disorder described herein, the amount of a compound or pharmaceutical composition which constitutes an “effective amount” may vary depending on the compound, the disorder and its severity, and the age of the subject to be treated.

### Compounds of the Present Invention

Provided herein are compounds of formula (I):

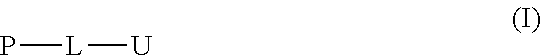

or a pharmaceutically acceptable salt thereof, in which:
P is a PAX3::FOXO1 fusion protein binding moiety;
L is a bivalent linker; and
U is an E3 ubiquitin ligase binding moiety.

The compounds of the present invention may be characterized as PROTACs, which are bifunctional small molecules that induce proximity between an E3 ubiquitin ligase and a target protein of interest, resulting in ubiquitination of the protein of interest and its subsequent degradation by a proteasome. Advantages associated with PROTACs include that they can deplete intracellular levels of the targeted protein, that they can work catalytically (i.e., can be recycled so that one PROTAC molecule can turn over multiple molecules of the protein of interest), and that target protein degradation by PROTACs can suppress resistant mutation and/or upregulation of the protein of interest [23]. In the present invention, the protein of interest is the PAX3::FOXO1 fusion protein.

As used herein, “PAX3::FOXO1 fusion protein,” “PAX3::FOXO1 protein,” and “PAX3::FOXO1” all refer to a fusion protein of PAX3 and FOXO1, and can be used interchangeably.

In the present invention, the PAX3::FOXO1 fusion protein binding moiety comprises a moiety that has demonstrated an ability to bind to the PAX3::FOXO1 fusion protein. Such an ability may be based on binding assays known in the art, for example, SPR [24], isothermal titration calorimetry [25], microscale thermophoresis [26], bio-layer interferometry [27], differential scanning fluorometry [28], fluorescence polarization [29], or nuclear magnetic resonance [30].

In embodiments of the invention, the PAX3::FOXO1 fusion protein binding moiety comprises an agent selected from Table 1, or a derivative thereof. In some embodiments, the PAX3::FOXO1 fusion protein binding moiety comprises a derivative of piperacetazine. Examples of derivatives of piperacetazine include, but are not limited to, C6.12 (CAS Registry No. 3733-38-8), C6.16 (CAS Registry No. 1186202-30-1), C6.21 (CAS Registry No. 58-38-8), C6.14, C6.15, C6.18a, and C6.18b. In certain embodiments, the PAX3::FOXO1 fusion protein comprises an agent selected from NSC697468, alcuronium chloride, NSC2805, didanosine, carboplatin, piperacetazine, C6.12, C6.16, C6.21, C6.14, C6.15, C6.18a, and C6.18b. In particular embodiments, the PAX3::FOXO1 fusion protein binding moiety comprises NSC697468, piperacetazine, C6.12, C6.15, or C6.18b.

In embodiments of the invention, the PAX3::FOXO1 fusion protein binding moiety may be a pharmaceutically acceptable salt of the agent selected from Table 1 or derivative thereof. For example, the PAX3::FOXO1 fusion protein binding moiety may be a pharmaceutically acceptable salt of C6.12, C6.16, C6.21, C6.14, C6.15, C6.18a, or C6.18b. In certain embodiments, the pharmaceutically acceptable salt may be HCl.

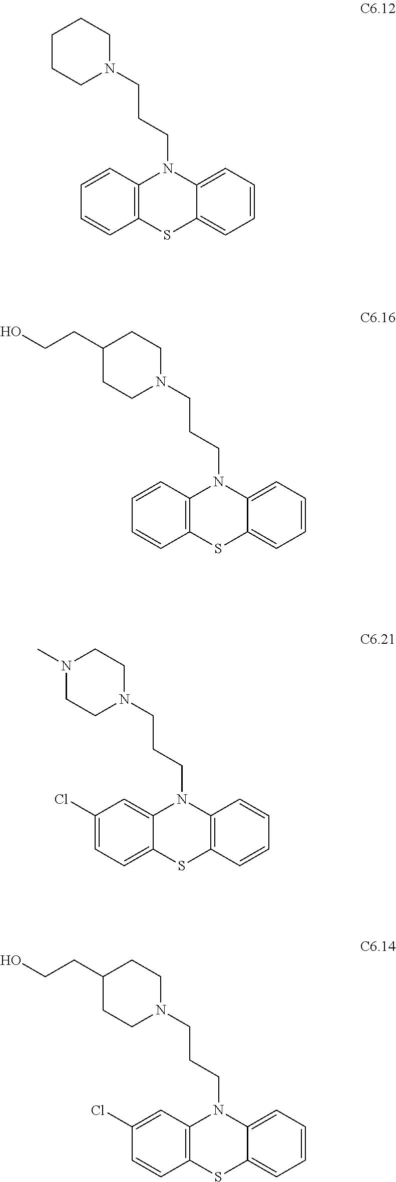 [SMILES: c1ccc2c(c1)Sc1ccccc1N2CCCN1CCCCC1 ; OCCC1CCN(CCCN2c3ccccc3Sc3ccccc32)CC1 ; CN1CCN(CCCN2c3ccccc3Sc3ccc(Cl)cc32)CC1 ; OCCC1CCN(CCCN2c3ccccc3Sc3ccc(Cl)cc32)CC1]

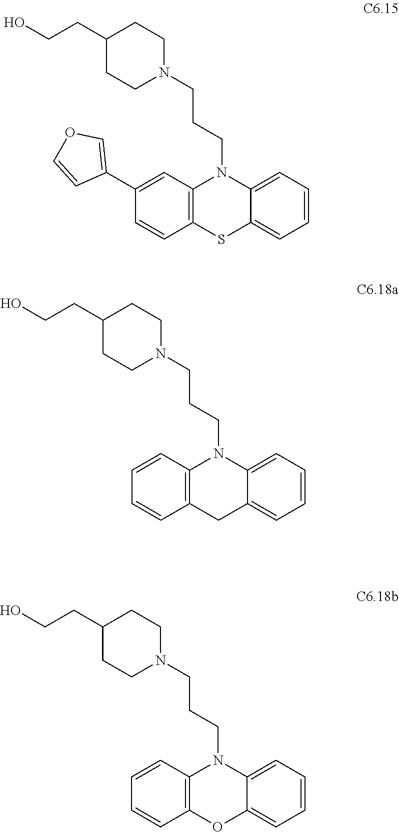 [SMILES: OCCC1CCN(CCCN2c3ccccc3Sc3ccc(-c4ccoc4)cc32)CC1 ; OCCC1CCN(CCCN2c3ccccc3Cc3ccccc32)CC1 ; OCCC1CCN(CCCN2c3ccccc3Oc3ccccc32)CC1]

The E3 ubiquitin ligase binding moiety comprises a ligand that binds to the substrate binding domain (SBD) of an E3 ubiquitin ligase. In the present invention, the E3 ubiquitin ligase binding moiety may comprise an E3 ubiquitin ligase ligand known in the art. In preferred embodiments, the E3 ubiquitin ligase binding moiety may comprise a E3 ubiquitin ligase ligand known in the art for use with PROTACs.

In some embodiments, the E3 ubiquitin ligase binding moiety may comprise a ligand that bind to an E3 ubiquitin ligase such as VHL, CRBN, CIAP, MDM2, or SKP-1.

Examples of VHL ligands for use in the present invention include, but are not limited to VH032, VH101, VH298, and derivatives thereof.

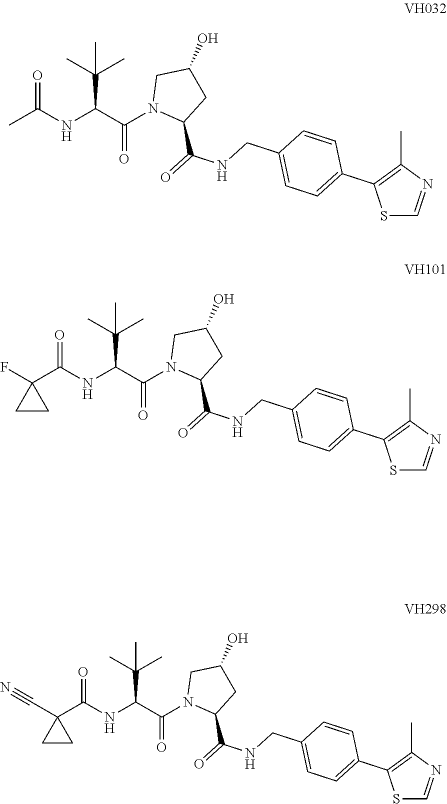 [SMILES: CC(=O)N[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)C2(F)CC2)C(C)(C)C)cc1 ; Cc1ncsc1-c1ccc(CNC(=O)[C@@H]2C[C@@H](O)CN2C(=O)[C@@H](NC(=O)C2(C#N)CC2)C(C)(C)C)cc1]

Examples of CRBN ligands for use in the present invention include, but are not limited to, thalidomide, pomalidomide, lenalidomide, 4-fluoro-thalidomide, and derivatives thereof.

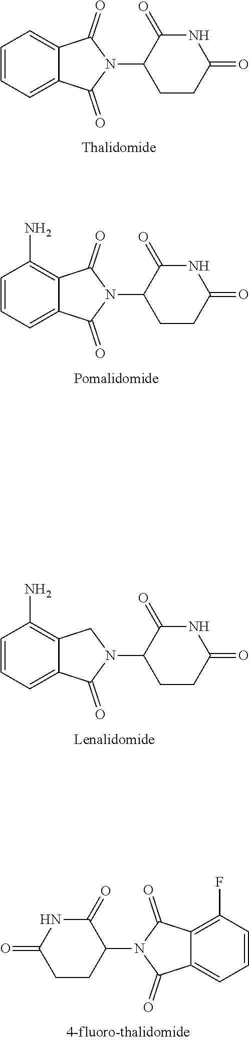 [SMILES: O=C1CCC(N2C(=O)c3ccccc3C2=O)C(=O)N1 ; Nc1cccc2c1C(=O)N(C1CCC(=O)NC1=O)C2=O ; Nc1cccc2c1CN(C1CCC(=O)NC1=O)C2=O ; O=C1CCC(N2C(=O)c3cccc(F)c3C2=O)C(=O)N1]

Examples of cIAP ligands for use in the present invention include, but are not limited to, methylbestatin and derivatives thereof, and LCL161 derivative.

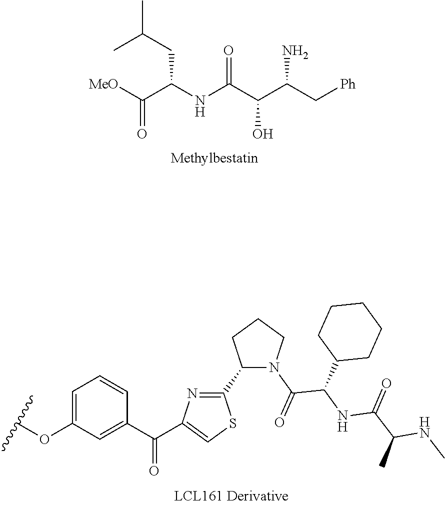 [SMILES: COC(=O)[C@H](CC(C)C)NC(=O)[C@@H](O)[C@H](N)Cc1ccccc1 ; *Oc1cccc(C(=O)c2csc([C@@H]3CCCN3C(=O)[C@@H](NC(=O)[C@H](C)NC)C3CCCCC3)n2)c1]

Examples of MDM2 ligands for use in the present invention include, but are not limited to, nutlin, idasanutlin, and derivatives thereof.

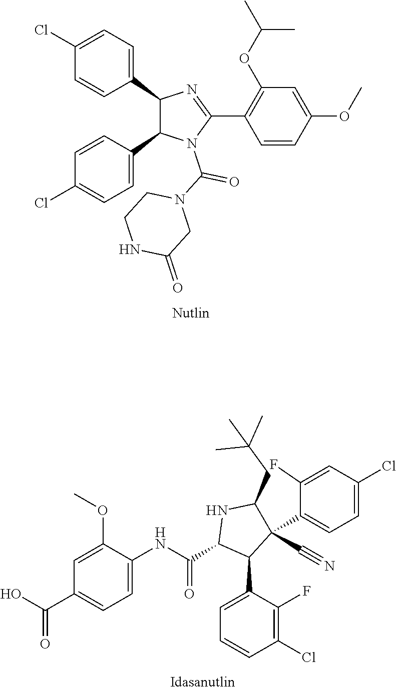 [SMILES: COc1ccc(C2=N[C@H](c3ccc(Cl)cc3)[C@H](c3ccc(Cl)cc3)N2C(=O)N2CCNC(=O)C2)c(OC(C)C)c1 ; COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

Examples of SKP-1 ligands for use in the present invention include, but are not limited to, EN884, and derivatives thereof. Examples of derivatives of EN884 include, but are not limited to, AD-5-49, AD-5-47a, and AD-5-47b [31].

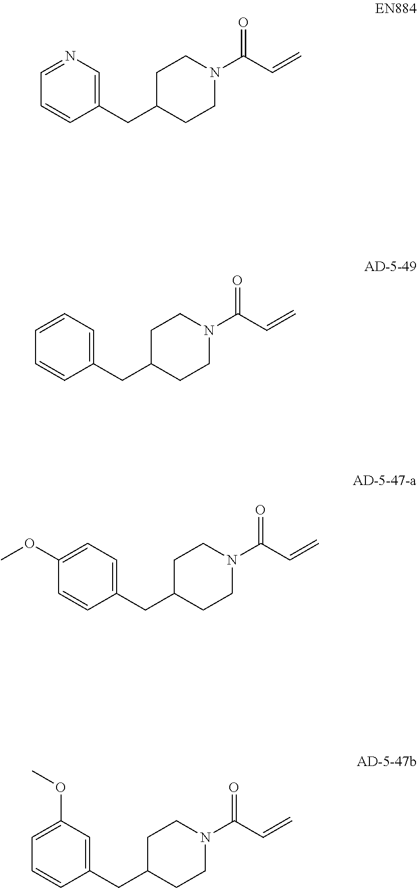

Additional examples of a E3 ubiquitin ligase binding moiety for use in the present invention include ligands that bind to Kelch-like ECH-associated protein 1 (KEAP1) such as bardoxolone methyl; ligands that bind to damage-specific DNA binding protein 1 (DDB1)-CUL4 associated factor 15 (DCAF15) such as indisulam, E7820, or chloroquinoxaline sulfonamide; and ligands that bind to RING-type zinc-finger protein 114 (RNF114) such as nimbolide.

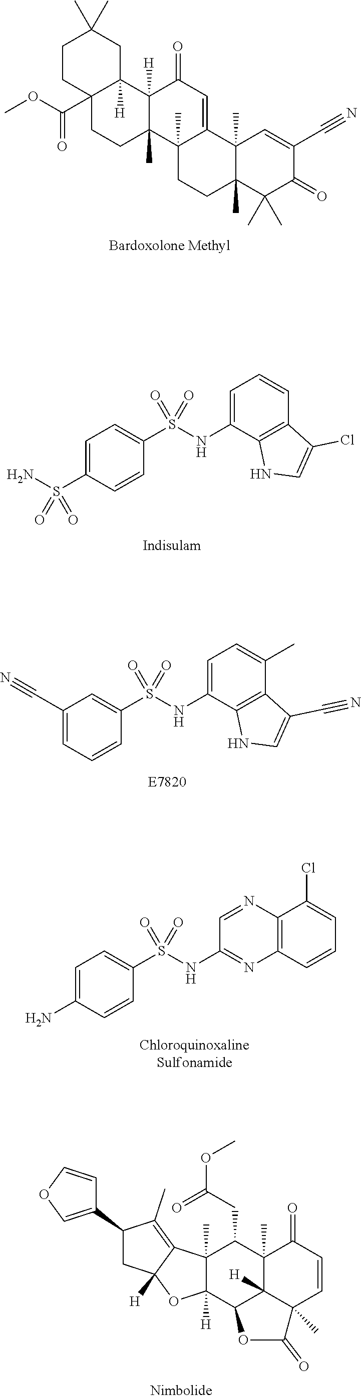

In particular embodiments, the E3 ubiquitin ligase binding moiety comprises 4-fluoro-thalidomide, pomalidomide, or AD-5-47a.

In the present invention, the bivalent linker may comprise a linker design known in the art. In preferred embodiments, the bivalent linker may comprise a linker design known in the art for use with PROTACs.

In some embodiments, the bivalent linker may comprise alkyl, PEG, and/or extended glycol chains. In certain embodiments, the linker may further comprise, or may alternatively comprise, an alkyne motif, triazole motif, piperazine motif, piperidine motif, or a combination thereof.

In some embodiments, the linker may be between about 3 and 50 atoms in length, or between about 5 and 40 atoms in length, or between about 7 and 30 atoms in length.

In certain embodiments, the bivalent linker may comprise a structure selected from the following:

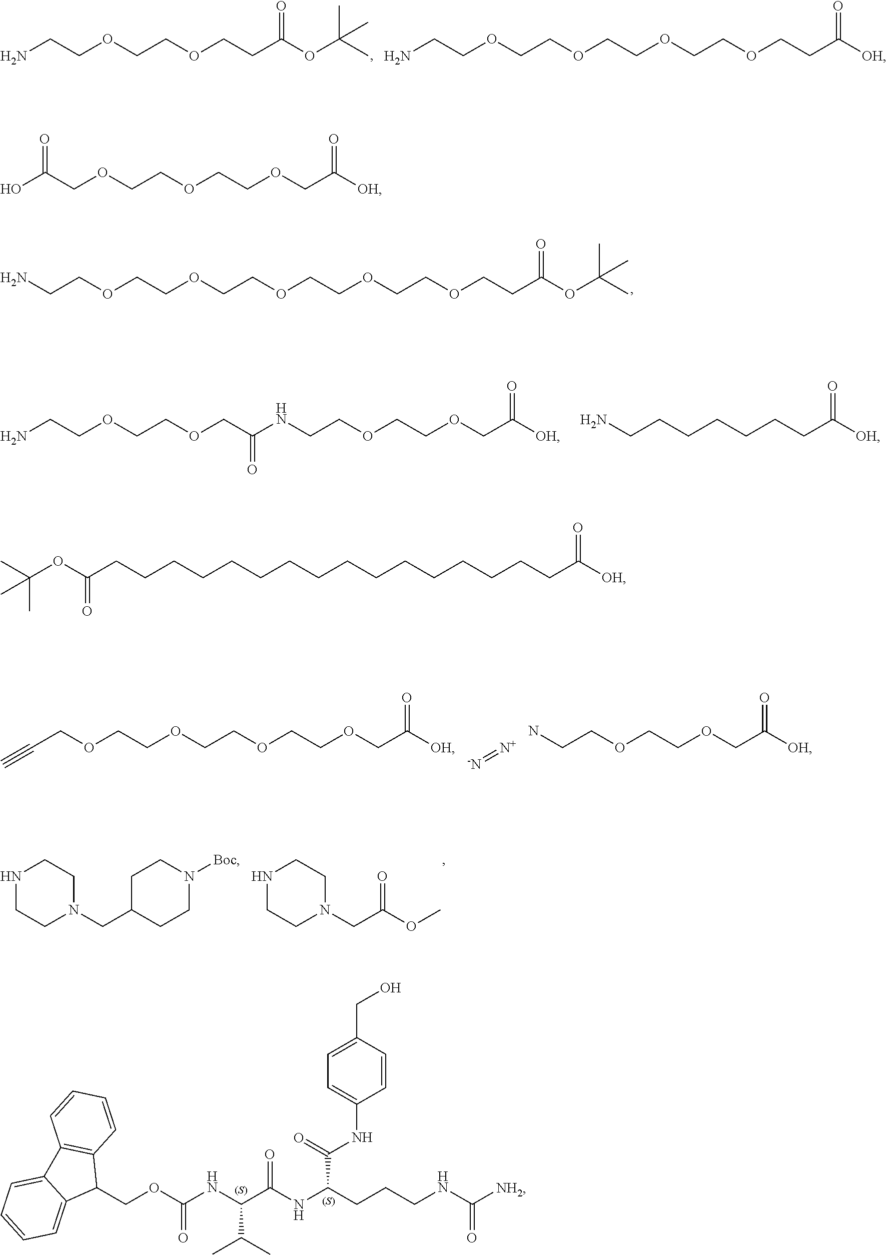

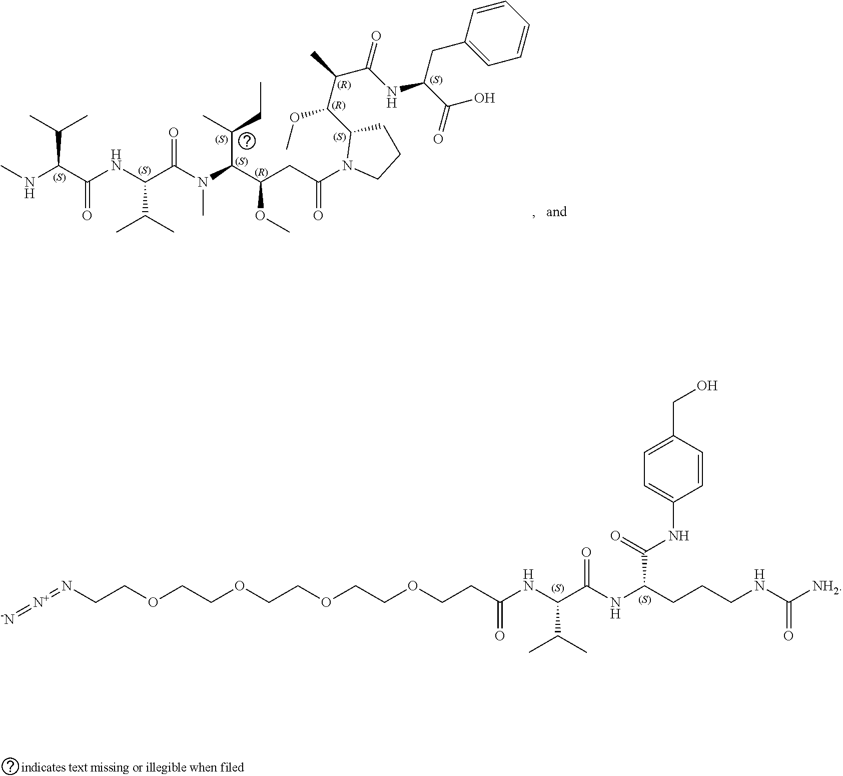

It is within the capability of one of ordinary skill in the art to design a bivalent linker for use in the present invention without undue experimentation.

In certain embodiments, the compound of the present invention comprises NSC697468 as the PAX3::FOXO1 fusion protein binding moiety, and pomalidomide as the E3 ubiquitin ligase binding moiety. In particular embodiments, the compound is selected from Compound A, Compound B, Compound C, Compound D, and Compound G.

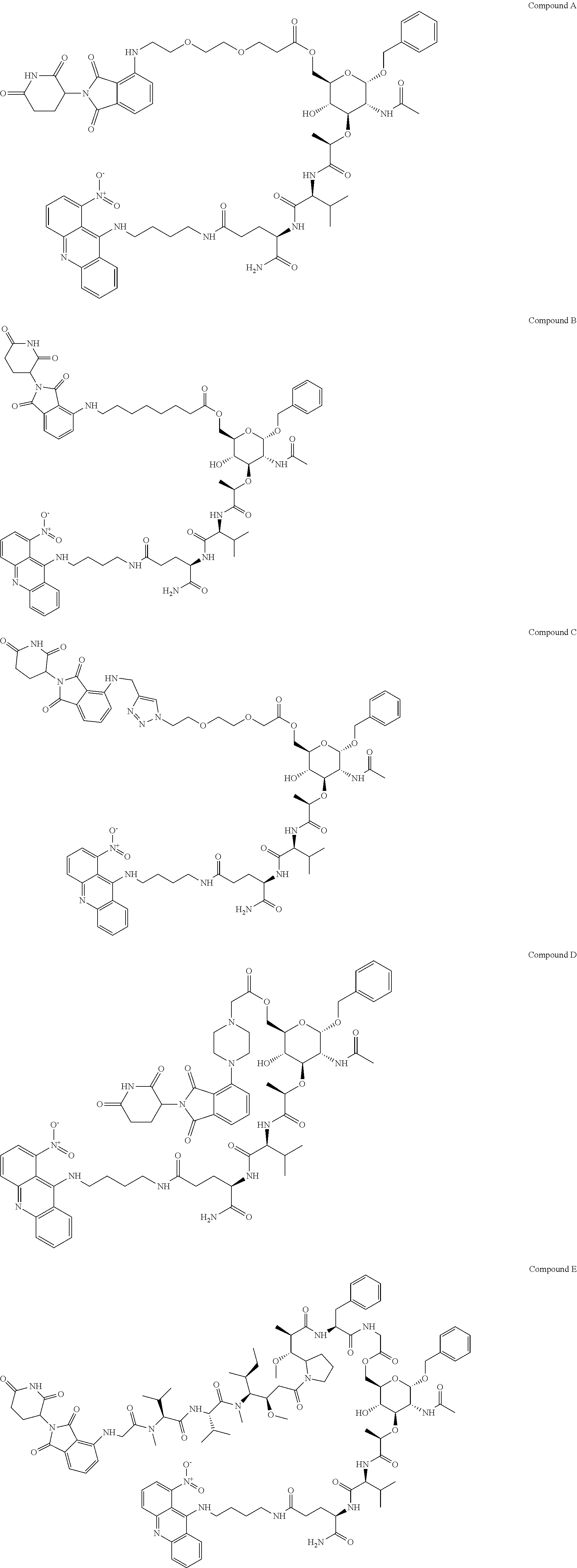

In certain embodiments, the compound of the present invention comprises C6.18b as the PAX3::FOXO1 fusion protein binding moiety, and pomalidomide as the E3 ubiquitin ligase binding moiety. In particular embodiments, the compound is selected from Compound 01-A1, Compound 01-B1, Compound 01-C1, Compound 01-D1, and Compound 01-E1.

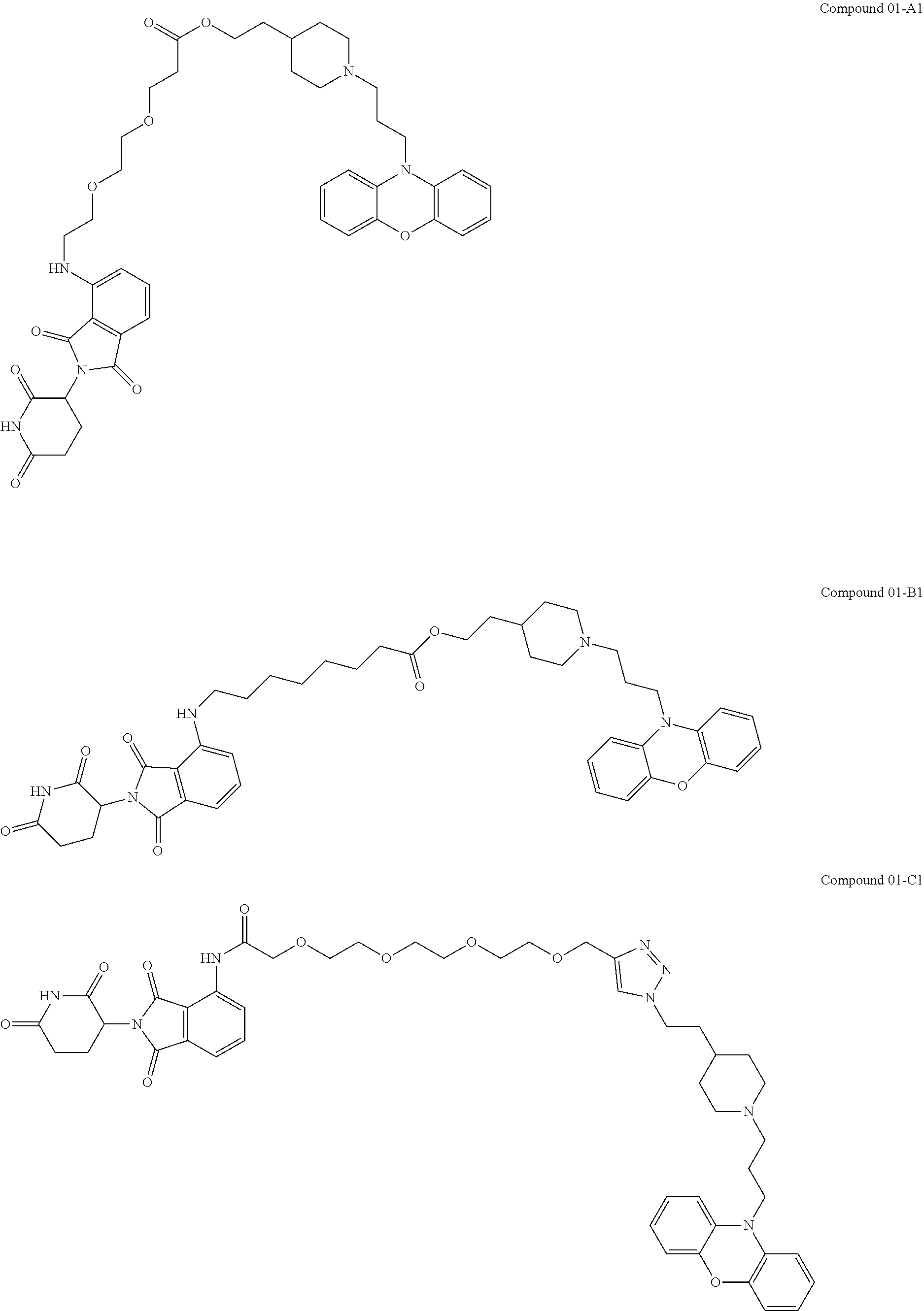 [SMILES: O=C1CCC(N2C(=O)c3cccc(NCCOCCOCCC(=O)OCCC4CCN(CCCN5c6ccccc6Oc6ccccc65)CC4)c3C2=O)C(=O)N1 ; O=C1CCC(N2C(=O)c3cccc(NCCCCCCCC(=O)OCCC4CCN(CCCN5c6ccccc6Oc6ccccc65)CC4)c3C2=O)C(=O)N1 ; O=C1CCC(N2C(=O)c3cccc(NC(=O)COCCOCCOCCOCc4cn(CCC5CCN(CCCN6c7ccccc7Oc7ccccc76)CC5)nn4)c3C2=O)C(=O)N1]

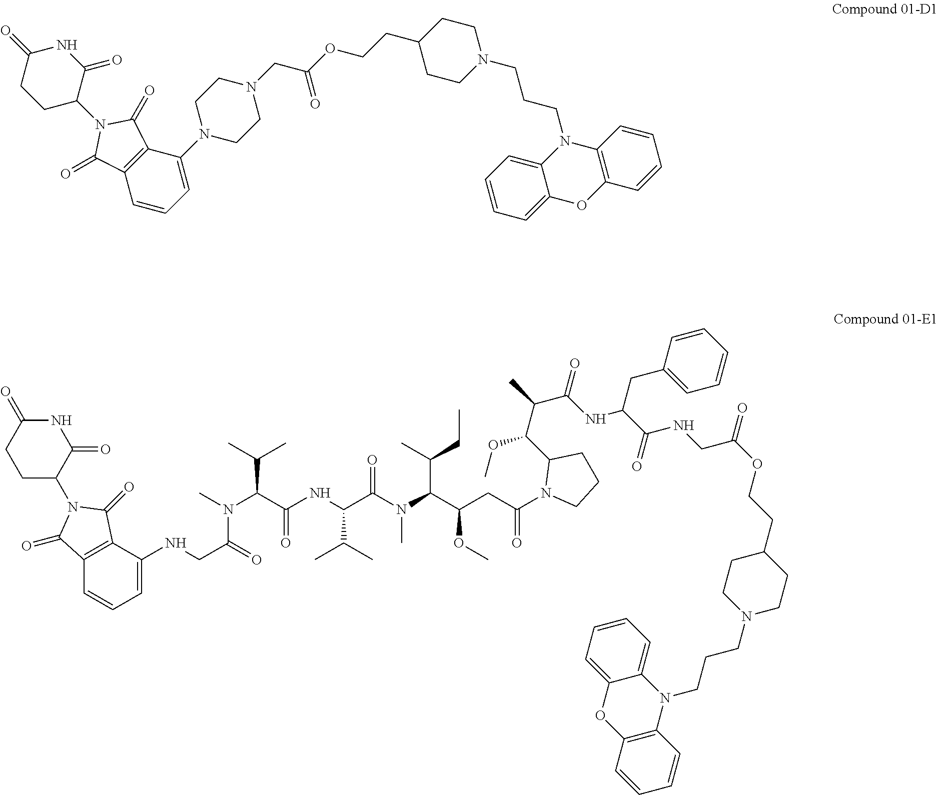 [SMILES: O=C1CCC(N2C(=O)c3cccc(N4CCN(CC(=O)OCCC5CCN(CCCN6c7ccccc7Oc7ccccc76)CC5)CC4)c3C2=O)C(=O)N1 ; CC[C@H](C)[C@@H]([C@@H](CC(=O)N1CCCC1[C@H](OC)[C@@H](C)C(=O)NC(Cc1ccccc1)C(=O)NCC(=O)OCCC1CCN(CCCN2c3ccccc3Oc3ccccc32)CC1)OC)N(C)C(=O)[C@@H](NC(=O)[C@H](C(C)C)N(C)C(=O)CNc1cccc2c1C(=O)N(C1CCC(=O)NC1=O)C2=O)C(C)C]

In certain embodiments, the compound of the present invention comprises a PAX3::FOXO1 fusion protein binding moiety selected from C6.12, C6.16, C6.21, C6.14, and C6.15; an alkyl linker; and pomalidomide as the E3 ubiquitin ligase binding moiety. In particular embodiments, the compound is selected from Compound 07-A, Compound 07-B, Compound 07-C, Compound 07-D, and Compound 07-E.

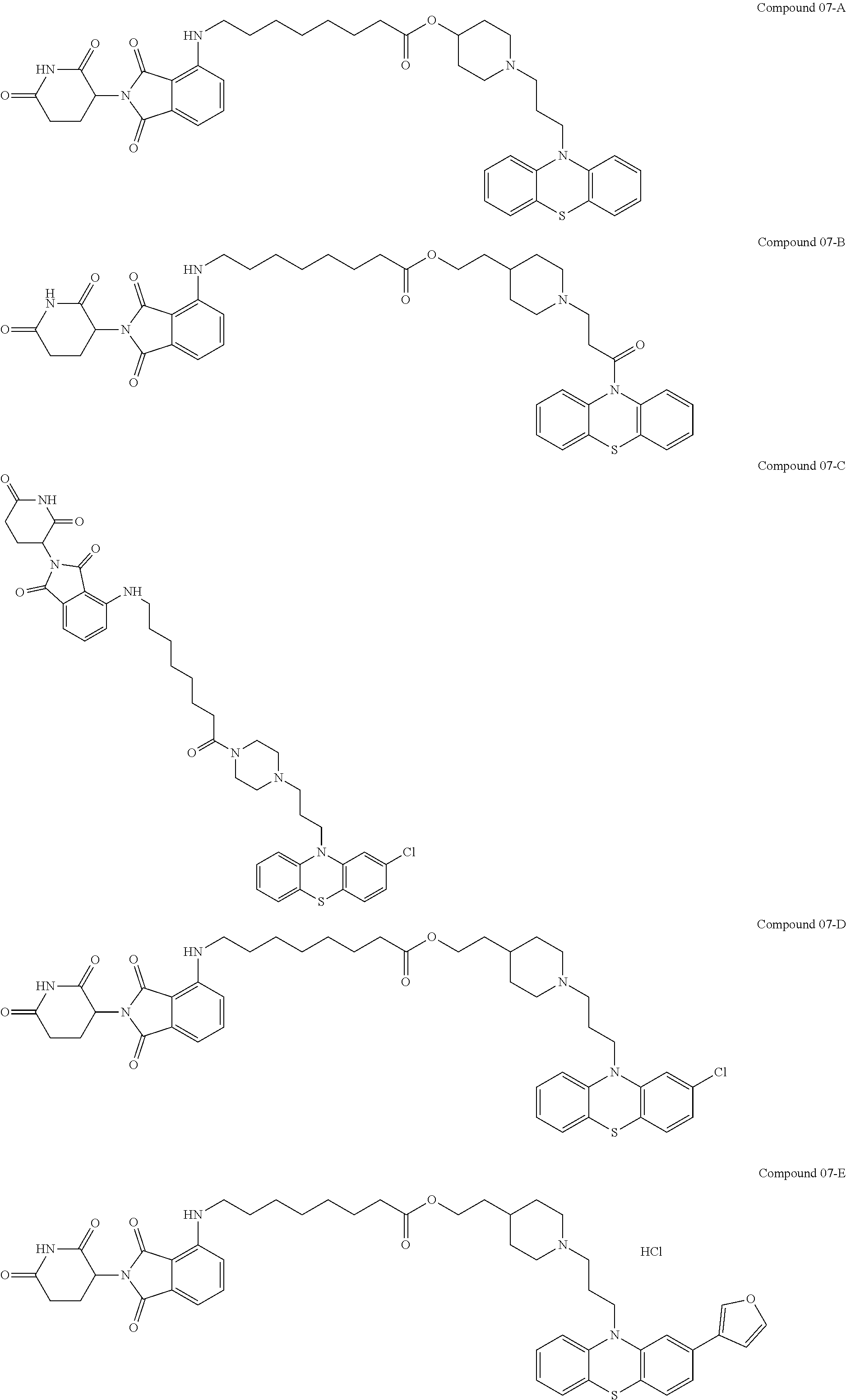

In certain embodiments, the compound of the present invention comprises a PAX3::FOXO1 fusion protein binding moiety selected from C6.12, C6.15, and C6.18b; and AD-5-47a as the E3 ubiquitin ligase binding moiety. In particular embodiments, the compound is selected from Compound G-3A, Compound G-3B, and Compound G-3C.

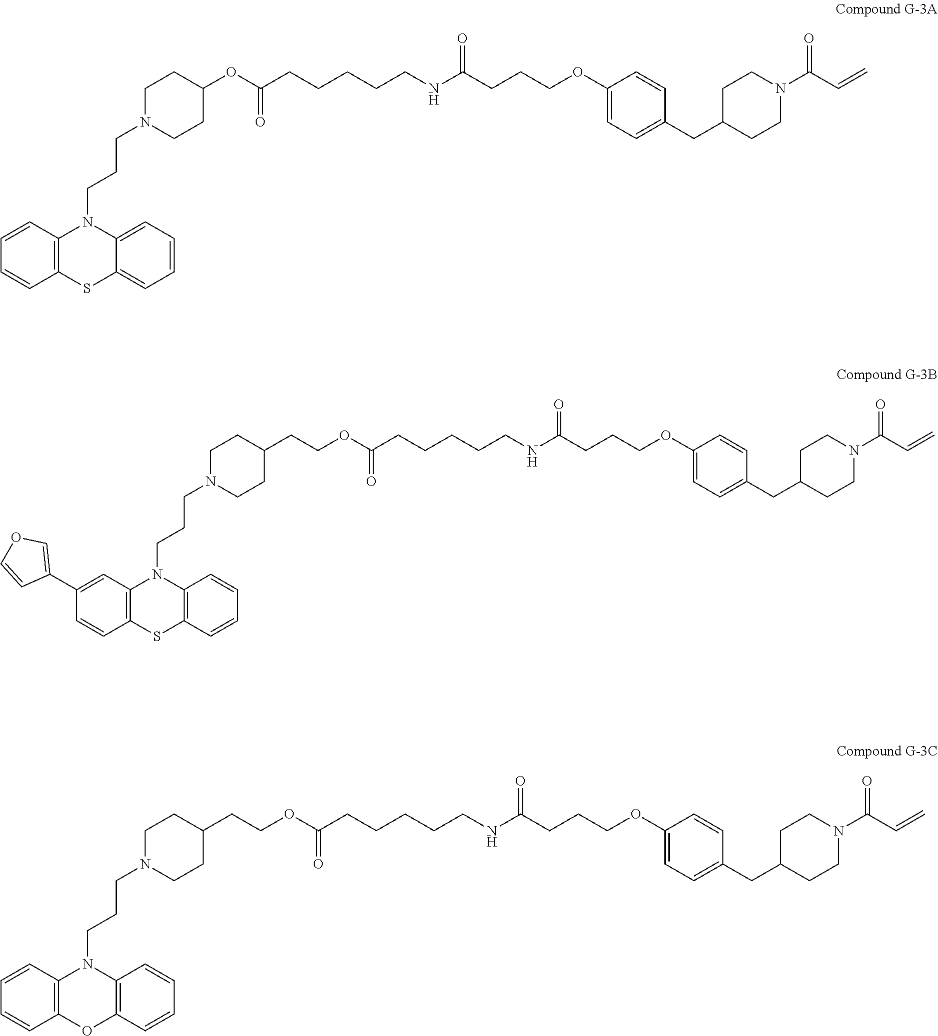 [SMILES: C=CC(=O)N1CCC(Cc2ccc(OCCCC(=O)NCCCCCC(=O)OC3CCN(CCCN4c5ccccc5Sc5ccccc54)CC3)cc2)CC1 ; C=CC(=O)N1CCC(Cc2ccc(OCCCC(=O)NCCCCCC(=O)OCCC3CCN(CCCN4c5ccccc5Sc5ccc(-c6ccoc6)cc54)CC3)cc2)CC1 ; C=CC(=O)N1CCC(Cc2ccc(OCCCC(=O)NCCCCCC(=O)OCCC3CCN(CCCN4c5ccccc5Oc5ccccc54)CC3)cc2)CC1]

In some embodiments, the compound of the present invention further comprises a cell-delivering moiety. As used herein, a “cell-delivering moiety” refers to any structure that facilitates the delivery of the compound or promotes transduction of the composition into a target cell. A cell-delivering moiety may be derived from a virus protein or peptide, or may be a hydrophobic compound capable of penetrating cell membranes.

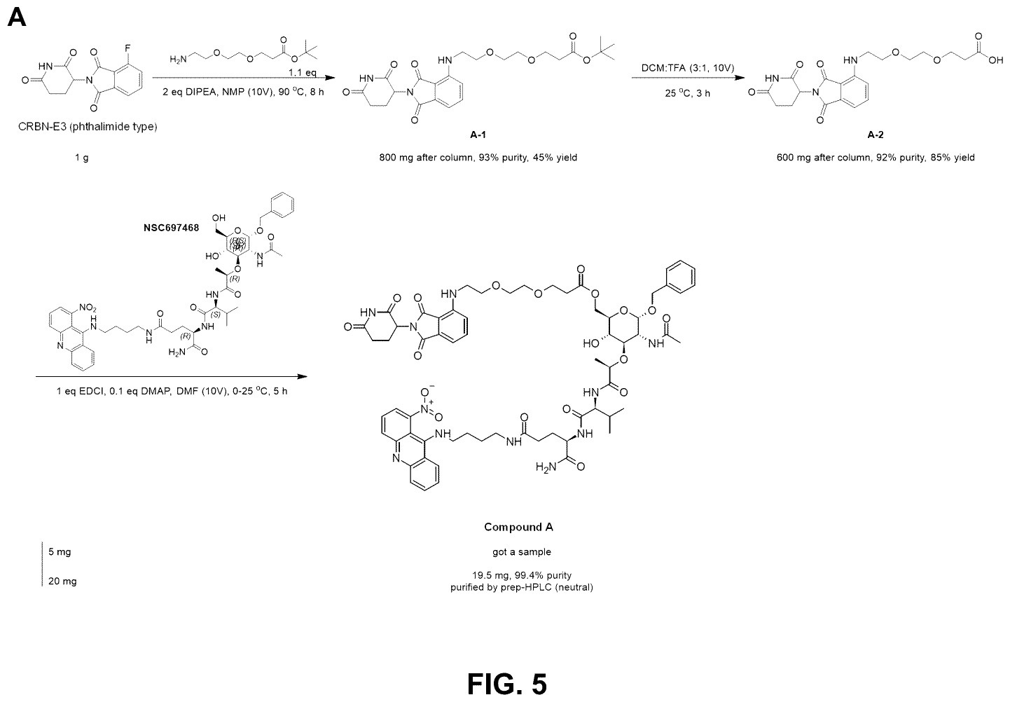

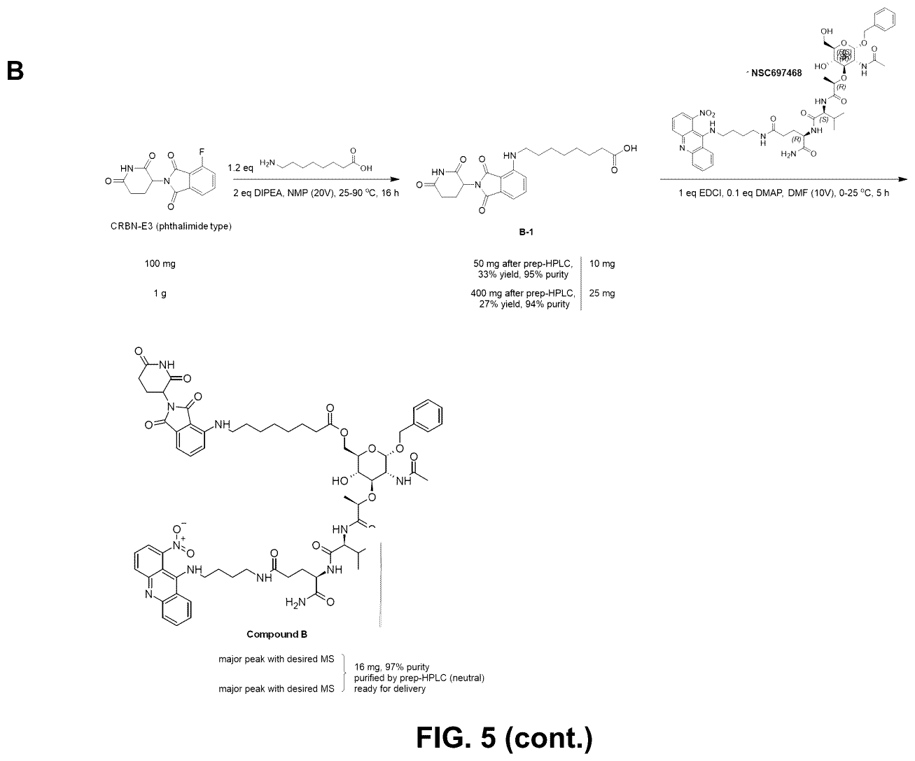

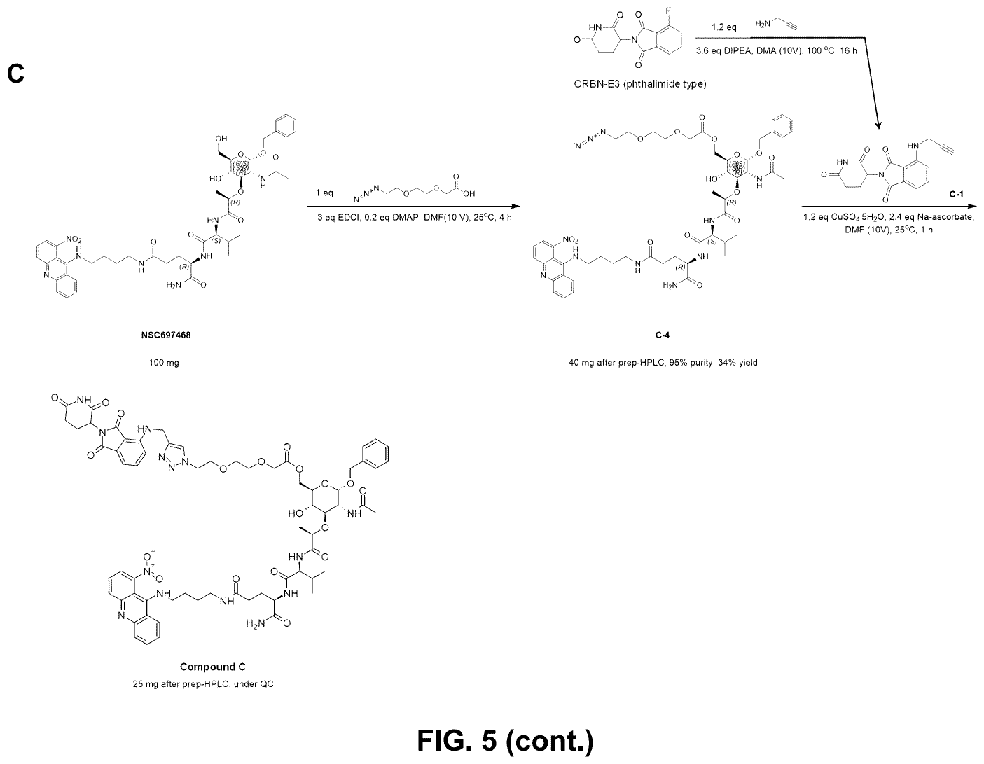

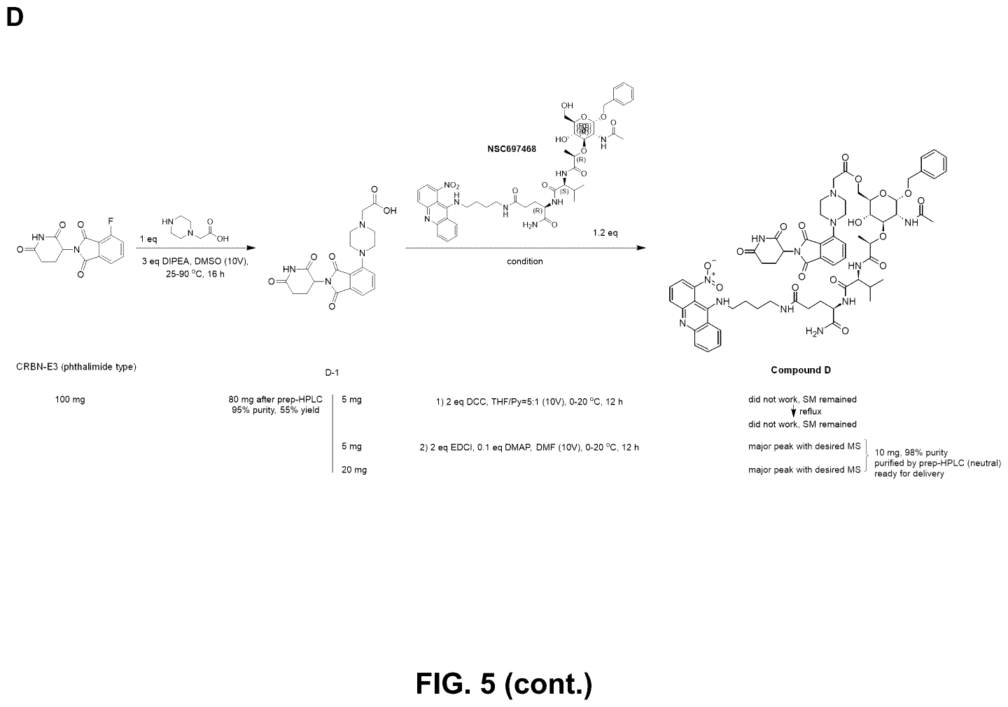

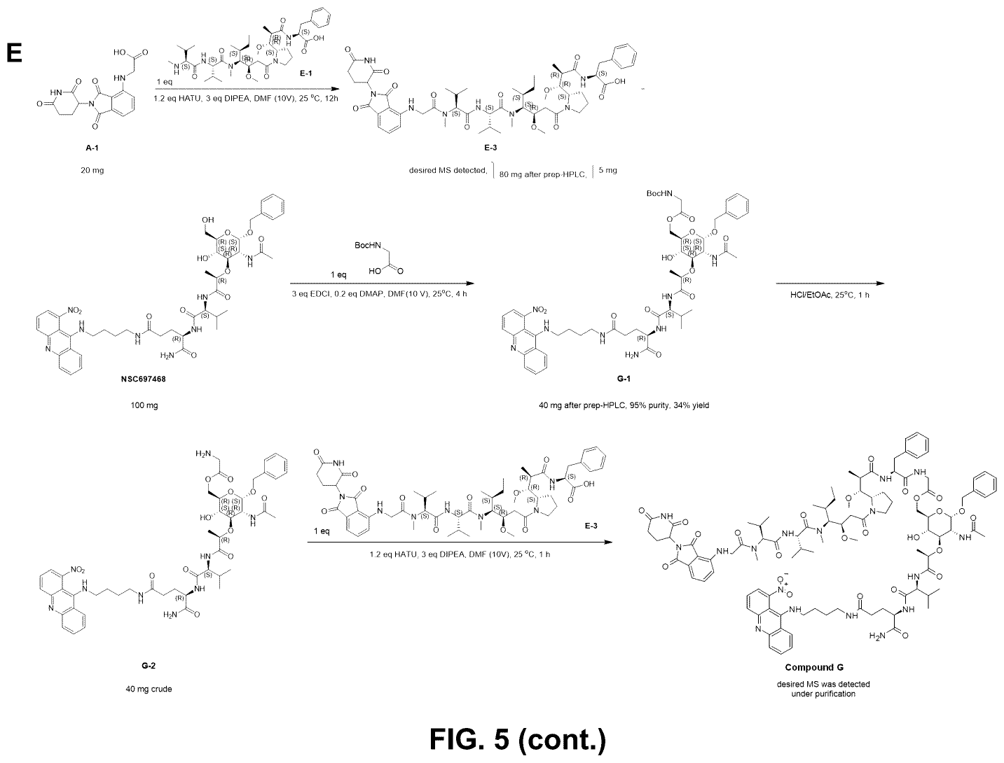

Synthesis of the compounds of the invention is within the purview of one of skill in the art. In particular, in certain embodiments, the compounds of the disclosure may be prepared by a modular “Click” chemistry-based approach. Synthesis of the compounds may also involve chemical modification of native side chains, incorporation of unnatural amino acids, or the use of enzymatic reactions or sequence tags. Examples of how compounds may be synthesized are demonstrated in Example 2 and shown in FIG. 5.

### Pharmaceutical Compositions, Kits, and Administration

In another aspect, the present invention provides a pharmaceutical composition comprising a compound of the present invention and a pharmaceutically acceptable carrier. Examples of a pharmaceutically acceptable carrier include, but are not limited to, water or physiological saline.

In some embodiments, the pharmaceutical composition may further comprise one or more diluents, excipients, or other additives. For example, the pharmaceutical composition may comprise one or more of a buffer (e.g., acetate, phosphate or citrate buffer), a surfactant (e.g., polysorbate), a stabilizing agent (e.g., polyol or amino acid), a preservative (e.g., sodium benzoate), and/or other conventional solubilizing or dispersing agents.

The pharmaceutical compositions may be formulated for oral, intravenous, intramuscular, subcutaneous, transdermal, or intraperitoneal administration routes of administration. In embodiments in which the pharmaceutical composition is formulated for oral administration, the pharmaceutical composition may be in the form of, for example, a tablet, capsule, suspension or liquid.

In a further aspect, the present invention provides kits comprising a compound of the present invention and/or pharmaceutical composition as provided herein and, optionally, instructions for use. The kit can further contain at least one additional reagent, and/or one or more additional active agent. Kits typically include a label indicating the intended use of the contents of the kit. In this context, the term “label” includes any writing or recorded material supplied on or with the kit, or that otherwise accompanies the kit.

The compounds of the present invention may be used in combination with one or more other treatments of diseases or conditions for which compounds of this disclosure or the other drugs may have utility. Examples of treatment include surgery, chemotherapy, radiation, and immunotherapy. In embodiments in which the treatment is chemotherapy, the chemotherapy may comprise a VAC regimen, consisting of vincristine, actinomycin D, and cyclophosphamide; or the IVA regimen, consisting of ifosfamide, vincristine, and actinomycin D.

### Uses of Compounds of the Present Invention

In one aspect, the compounds and/or pharmaceutical compositions of the present invention is for use in reducing PAX3::FOXO1 protein levels in a subject with FP-RMS. Accordingly, an aspect of the present invention provides methods of reducing PAX3::FOXO1 protein levels in a subject with FP-RMS. These methods may comprise administering to the subject a compound or a pharmaceutical composition of the present invention. In some embodiments, the subject has FP-ARMS.

The PAX3::FOXO1 protein levels may be measured as a concentration of PAX3::FOXO1 protein in a biological sample from the subject, such as from the blood or urine of the subject, or a tissue or cell sample, such as from the RMS tumor, using protein measurement methods known in the art (e.g., Western blot analysis, immunohistochemistry, flow cytometry, enzyme linked immunosorbent assays, etc.). The PAX3::FOXO1 protein level may be reduced from a level previously measured in the subject, such as before administration of the compounds and/or pharmaceutical compositions of the present invention.

In another aspect, the compounds and/or pharmaceutical compositions of the present invention is for use in ubiquitinating a PAX3::FOXO1 fusion protein in a cell. In a further aspect, the present invention provides methods of ubiquitinating a PAX3::FOXO1 fusion protein in a cell. The method may comprise administering a compound or a pharmaceutical composition of the present invention to the cell. In some embodiments, the cell is a FP-RMS tumor cell. In certain embodiments, the cells is a RP-ARMS tumor cell.

In another aspect, the compounds and/or pharmaceutical compositions of the present invention is for use in treating FP-RMS in a subject in need thereof. Accordingly, an aspect of the present invention provides methods of treating FP-RMS in a subject in need thereof. These methods may comprise administering to the subject a compound or a pharmaceutical composition of the present invention. In some embodiments, the FP-RMS is FP-ARMS.

In yet another aspect, the compounds and/or pharmaceutical compositions of the present invention is for use in treating a disease caused by the overexpression of PAX3::FOXO1 protein in a subject in need thereof. Accordingly, a further aspect of the present invention provides methods of treating a disease caused by the overexpression of PAX3::FOXO1 protein in a subject in need thereof. The method may comprise administering to the subject a compound or a pharmaceutical composition of the present invention. In some embodiments, the disease is biphenotypic sinonasal sarcoma.

Response to administration of the compounds and/or pharmaceutical composition may compare one or more measures of efficacy after a treatment regimen, as compared to baseline, e.g., prior to administration. A baseline assessment is preferably performed within 24, 48, or 72 hours, or within one, two, three, or four weeks prior to the first administration. In certain embodiments, a baseline assessment is performed within 24 hours prior to the first administration.

Efficacy of treatment can be evaluated by one or more known measures. For example, subjects administered the compounds and/or pharmaceutical composition of the present invention can experience outcomes including extended survival, improved progression-free survival, improved duration of response, longer remission, reduced risk of relapse, and/or improved tumor response to treatment with the combination therapy of the invention, compared with the same outcome(s) in subjects not subjected to methods of the invention, i.e., control subjects. An outcome in a subjects treated by a method of the invention can be compared, for example, to the median outcome in a population of control subjects. The population of control subjects can be administered, for example, a regimen selected from the group consisting of a placebo, surgery, radiation, chemotherapy, immunotherapy, hormone-based therapy, or targeted therapy.

“Tumor burden” is the total mass or total size of cancerous tissue in a subject's body. Tumor response can be evaluated by measures including objective response rate, disease control rate, and duration of response. These parameters can be determined, for example, by revised Response Evaluation Criteria in Solid Tumors (RECIST 1.1) [32]. Objective response rate assesses reduction of tumor size, for example, tumor diameter, which can be determined by clinical examination and/or imaging. Where a subject has multiple tumors, tumor size can optionally be expressed as the average diameter of all tumors or by the sum of diameters of all tumors. Superficial tumors can be measured clinically, for instance, using calipers or by photography and ruler measurement. Imaging methods include computed tomography (CT), typically with contrast; X-ray; magnetic resonance imaging (MRI); and positron emission tomography (PET), such as (18) F-fluorodeoxyglucose PET. In some embodiments, CT is utilized to assess tumor response. In other embodiments, MRI, such as gadolinium-enhanced MRI, is utilized to assess tumor response. Accordingly, in one aspect, the invention provides a method of reducing tumor burden, i.e., tumor mass and/or tumor size, in a subject, the method comprising administering to the subject a compound and/or pharmaceutical composition of the present invention. Reduction in tumor burden is measured relative to baseline.

In certain embodiments, particularly those in which assessment is by RECIST 1.1, disease control rate defines the level of tumor response as complete response (CR), which is the disappearance of tumor(s); partial response (PR), which is a decrease of at least 30%, in the size of tumor(s); stable disease, in which there is no change in the size of tumor(s); or disease progression, which is an increase of at least 20%, in tumor size and/or new lesions.

Duration of response is the length of time from the achievement of a response until disease progression, i.e., the period in which a tumor does not grow or spread, or death. Duration of response in subjects receiving combination therapy of the invention can be, for example, at least 4, 6, 8, 10, or 12 weeks, at least 4, 6, 8, 10, 12, 16, 18, or 24 months, or at least 3, 4, or 5 years. Accordingly, in one aspect, the invention provides a method of increasing the duration of response in a subject, the method comprising administering to the subject a compound and/or pharmaceutical composition of the present invention. Increase in duration of response is measured relative to the median duration of response in a control population.

Survival can be assessed as overall survival, i.e., the length of time a subject lives, or as progression-free survival, i.e., the length of time a subjects is treated without progression or worsening of the disease. Survival can be measured from the date of diagnosis or from the date that treatment commences. Overall survival, median overall survival, progression-free survival, and median progression-free survival can be calculated, for example, by Kaplan-Meier analysis, based on the response to treatment. Accordingly, in one aspect, the invention provides a method of increasing overall survival in a subject, the method comprising administering to the subject a compound and/or pharmaceutical composition of the present invention. Increase in overall survival is measured relative to the median overall survival in a control population. In another aspect, the invention provides a method of increasing progression-free survival in a subject, the method comprising administering a compound and/or pharmaceutical composition of the present invention. Increase in progression-free survival is measured relative to the median progression-free survival in a control population.

In some embodiments, a subject is successfully treated according to the methods of the invention if the subject experiences or displays at least one of the following outcomes after administration of a compounds and/or pharmaceutical composition of the present invention:
(i) undetectability of the tumor (or at least one tumor, if multiple tumors are present at baseline);
(ii) at least about 10%, 20%, 30%, 40%, 50%, 60%, 70%, 80%, or 90% reduction in tumor size compared to baseline;
(iii) no significant increase (e.g., less than 20%) in tumor size compared to baseline;
(iv) significantly increased duration of response, optionally compared with median duration of response of a population of control subjects;
(v) significantly increased progression-free survival, optionally compared with median progression-free survival of a population of control subjects;
(vi) significantly increased overall survival, optionally compared with median overall survival of a population of control subjects.

The compound or pharmaceutical composition of the present invention may be administered in an effective amount.

In embodiments of the invention, administration of the compound or pharmaceutical composition of the present invention may be in vitro, ex vivo, or in vivo.

In embodiments in which the compound or pharmaceutical composition is administered to a subject, the compound or pharmaceutical composition may be administered, for example, orally, intravenously, intramuscularly, subcutaneously, transdermally, or intraperitoneally.

### EXAMPLES

### Example 1

A study was conducted that screened small molecule libraries to identify compounds that were capable of directly binding to PAX3::FOXO1 protein using surface plasmon resonance technology [33].

### Methods

### Chemicals

Unless stated otherwise, all chemicals and reagents were purchased from MilliporeSigma. Compound libraries for screening were obtained from the Developmental Therapeutics Program (DTP) of the National Cancer Institute and from the Prestwick Chemicals. The following compounds selected for further study were commercially obtained: carboplatin and didanosine (SelleckChem catalog nos. S1215 and S1702), alcuronium chloride (MilliporeSigma catalog no. 15180-03-07), piperacetazine (MedChemExpress catalog no. HY-B1152), and NSC2805 (Glixx Labs).

### PAX3::FOXO1 Purification

Full-length PAX3::FOXO1 (GenBank accession code: AAC50053.1) was cloned into pET104.1 DEST plasmid with a carboxy terminal 12 His tag and an amino terminal internal HA tag. Recombinant protein expression was induced by isopropyl-β-D-thiogalactopyranoside in Escherichia coli strain BL-21 (DE3). Cell lysates were prepared using the BugBuster® MasterMix (MilliporeSigma catalog no. 71456-4). Purification was done using a HiTrap Chelating HP 1 mL column in AKTA Pure 25 Explorer (Cytiva). The column was washed with water, charged with 100 mmol/L nickel sulfate and washed again with water. The cell pellet was dissolved in the running buffer (20 mmol/L sodium phosphate buffer pH 7.4, containing 500 mmol/L NaCl, 40 mmol/L imidazole), which was also used for equilibrating the column before sample application and consequent wash. Recombinant protein was eluted from the column with a linear gradient to running buffer containing 1 mol/L imidazole.

### SPR

Recombinant PAX3::FOXO1 and negative control CD99 proteins were immobilized onto a Biacore CM5 sensor chip by amine coupling in a Biacore 4000 instrument (Cytiva Life Sciences). Proteins were immobilized on the same CM5 chip in neighboring spots of the same flow cell at two different surface densities. High-density (HD) PAX3::FOXO1 surfaces had 2,700-4,200 RU protein. Low-density (LD) PAX3::FOXO1 surfaces had 400-700 RU protein. HD CD99 surfaces had 1,800-3,200 RU protein. LD CD99 surfaces had 500-800 RU protein. The theoretical binding capacity of Rₘₐₓ was calculated for each compound based on the amount of ligand captured on each spot and the molecular weights of each compound (Rₘₐₓ=R_{L}×MW of analyte/MW of ligand). Rₘₐₓ calculations were made with a 1:1 binding expectation. Compounds were injected individually at 50 μmol/L, and the binding sensorgrams were recorded. A double reference subtraction method was used to eliminate any bulk refractive index contribution to the measured binding signal, which was achieved by having an empty reference spot without any protein in all four flow cells of the CM5 chip and by injecting buffer alone over all surfaces. The signal coming from the empty reference spots and the buffer alone injections were subtracted from all compound signals to account for background noise. A solvent correction was applied to the sensorgrams to account for possible minor mismatch in DMSO content in between running buffer and small-molecule dilutions in the running buffer. Compounds were eliminated from further consideration if they (i) gave a higher signal on LD surface than HD surface for PAX3::FOXO1, (ii) bound to the HD PAX3::FOXO1 surface with greater than 200% of the expected Rₘₐₓ value, or (iii) bound to the HD PAX3::FOXO1 surface with less than 50% of the expected Rₘₐₓ value. The remaining compounds were compared for their binding signal on an HD PAX3::FOXO1 surface with an HD CD99 surface. Because the expected Rₘₐₓ value for each analyte is proportional to the molecular weight ratios and the surface density, the binding values were first normalized and expressed as % of Rₘₐₓ. Then, the % of Rₘₐₓ of PAX3::FOXO1 binding was divided by % of Rₘₐₓ of CD99 binding, and the compounds were ranked on the basis of this fold ratio. Any compound that showed 5-fold or higher binding to PAX3::FOXO1 was considered a primary hit. Validation binding experiments with small molecules and DNA were done in a Biacore T-200 system. PAX3::FOXO1 and EWS::FLI1 were immobilized on CM5 chips via standard amine coupling reaction. PBS-P (20 mmol/L phosphate buffer at pH 7.4, 137 mmol/L NaCl, 2.7 mmol/L KCl, 0.05% v/v surfactant P20) was used as the immobilization running buffer. For small-molecule studies, PBS-P was supplemented with 5% v/v DMSO. The following oligonucleotides were used in SPR studies:

For PAX3::FOX01:
(SEQ ID NO: 1)
5′-ACCGTGACTAATTTAATTAGTCACG-3′
(SEQ ID NO: 2)
3′-CGTGACTAATTAAATTAGTCACGGT-5′
For EWS::FLI1:
(SEQ ID NO: 3)
5′-ACCGGAAGGAAGGAAGGAAGGAAGGAAGGAAGTG-3′
(SEQ ID NO: 4)
3′-TGGCCTTCCTTCCTTCCTTCCTTCCTTCCTTCAC-5

### Cellular Thermal Shift Assay (CETSA)

Collected cell lysates were treated with 30 μmol/L piperacetazine or DMSO (negative control) for 30 minutes at room temperature and heated for 3 minutes at the indicated temperatures, followed by a 3-minute incubation at room temperature. The samples were then centrifuged at 16,000×g for 30 minutes at 4° C. Protein levels in the remaining supernatant were evaluated by immunoblotting. Quantification was done using Image Studio Lite (LI-COR Biosciences).

### Cell Culture

Murine U66788, U48484, U37125, and U57810 RMS cells were originally established from transgenic mouse models of ARMS and ERMS and grown in DMEM supplemented with 10% FBS. The RH28 cell line used in this study was a subclone selected for resistance to L-PAM. RH28, RD (ATCC catalog no. CCL-136), HEK293T (ATCC catalog no. CCL-3216), K7M2, K12, U2OS, MG63.3, HeLa (ATCC catalog no. CCL-2, and SN12C cells were grown in DMEM supplemented with 10% FBS. STA-ET-7.2, RH30 (ATCC catalog no. CRL-2061), and RH41 cells were grown in RPMI supplemented with 10% FBS, as well as A4573, which was additionally supplemented with 1% HEPES. Dbt-MYCN/indP3F cells were grown in Ham's/F-10 medium supplemented with 15% FBS, 50 μg/ml uridine, 1 mmol/l sodium pyruvate, and 1 mmol/L creatine. Cell lines were routinely tested for the presence of Mycoplasma using the MycoAlert system according to manufacturer's instructions (Lonza catalog no. LT07-318) and fingerprinted for identity confirmation.

### Luciferase Reporter Assays

Stable RH30 PAX3::FOXO1-responsive 6XPRS-Luc and SN12C PGK-responsive luciferase reporter cells were seeded in 96-well plates at 10,000 cells per well and allowed to grow for 24 hours. Cells were treated with test compounds at a concentration of 10 μmol/L in DMSO. Luciferase activity was measured after 48 hours of treatment. In a separate assay, cell lines were transfected with a pGL3-PDGFRA PAX3::FOXO1-responsive luciferase construct using X-tremeGENE 9 DNA Transfection Reagent (Roche catalog no. XTG9-RO) 24 hours prior to seeding, then seeded in 96-well plates at 5,000-40,000 cells per well and treated with piperacetazine or vehicle control. Luciferase activity was measured after 48 hours of drug treatment. HEK293T cells were used in a third luciferase assay, in which cells were transfected with PAX3::FOXO1-responsive pGL4.19-ASS1P luciferase reporter and either an empty vector or PAX3::FOXO1 construct, then treated with DMSO or piperacetazine. After 48 hours, the cells were lysed for the luciferase assay, and the same number of cells were lysed for protein quantification via Pierce bicinchoninic acid assay (Thermo Fisher Scientific catalog no. 23225). The luciferase assay readouts were then normalized to the total protein amounts. In all experiments, luciferase activity was quantified using a luciferase assay system (Promega catalog no. E1501) according to manufacturer's instructions.

### Immunoblotting

Immunoblotting was performed using anti-FOXO1 (Cell Signaling Technology catalog no. 2880), MYOD1 (Cell Signaling Technology catalog no. 13812), MYOG (Novus catalog no. NB100-56510), B7-H3 (CD276) (Cell Signaling Technology catalog no. 14058), PAX3 (Cell Signaling Technology catalog no. 12412), phospho-FOXO1 (pSer256; Cell Signaling Technology catalog no. 9461), and actin-HRP (Abcam catalog no. ab20272) antibodies. Whole-cell lysates from cells grown to near confluence were subjected to SDS-PAGE and transferred to a Low Fluorescence polyvinylidene difluoride Membrane (Bio-Rad catalog no. 1704275). The membranes were then subject to blocking in 5% nonfat dry milk in 1×TTBS (Tween-Tris-buffered saline; 20 mmol/L Tris-HCl, pH 7.5, 150 mmol/L NaCl, 0.5% Tween 20) for 1 hour. Primary antibodies were added to the membrane in 5% BSA in 1×TTBS for 2 hours. The membrane was then washed three times in 1×TTBS, and horseradish peroxidase-linked anti-rabbit or anti-mouse secondary antibody (Thermo Fisher Scientific catalog no. NA934; catalog no. NA9311ML) in 5% nonfat dry milk was added for one hour. The blots were then washed three times in 1×TTBS and developed using Millipore Immobilon Western chemiluminescent horseradish peroxidase substrate per the manufacturer's instructions (Millipore catalog no. WBKLS0500). Chemiluminescence was detected using a LI-COR Odyssey Fc imaging system. Densitometry values were generated using Image Studio Lite software.

### Cell Viability Assays

Cells were seeded on 96-well plates at a density of 5,000-10,000 cells/well in 100 μL of media. Piperacetazine or vehicle control were added after 24 hours, and cell viability was measured at 48 hours posttreatment using the CellTiter-Blue assay and measuring fluorescence following manufacturer's instructions (Promega catalog no. G808A) on a BioTek Synergy H4 plate reader.

### Statistical Analysis

Statistical analysis was performed using GraphPad Prism version 9.0. Statistical significance was defined as p<0.05.

### Results

Small Molecules Binding to PAX3::FOXO1 Protein were Identified by SPR.

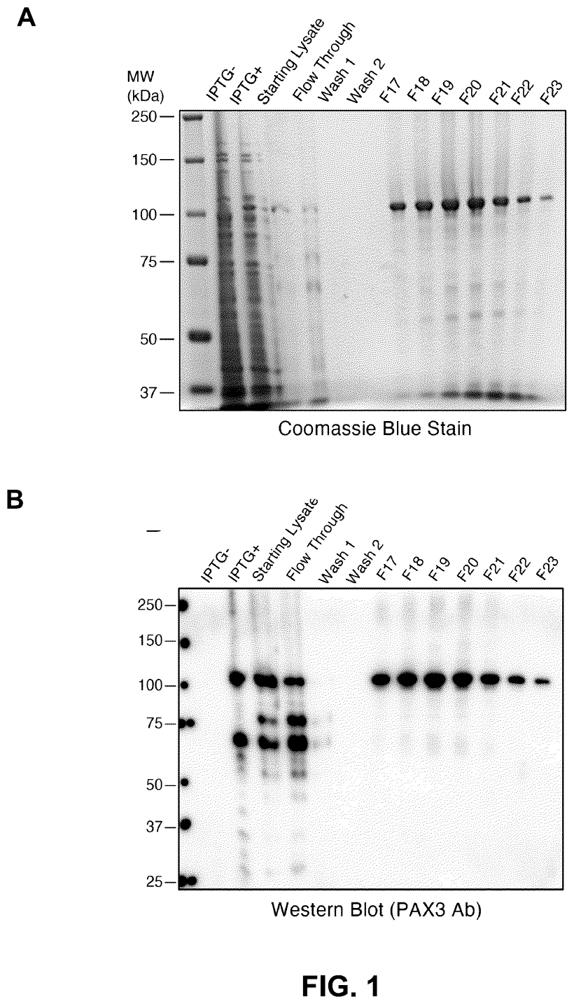

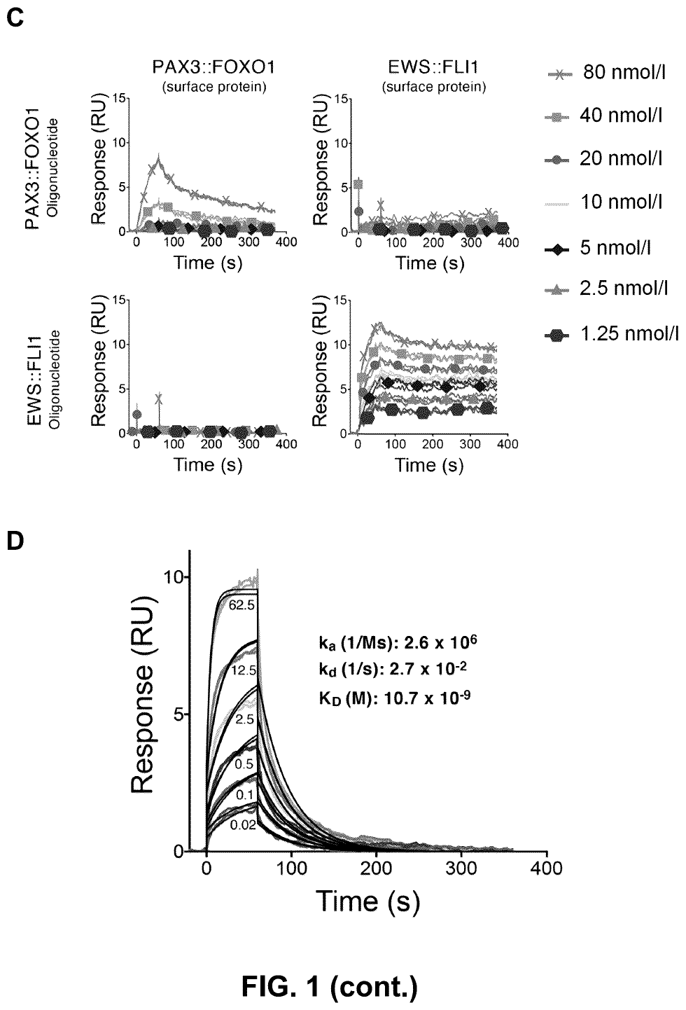

To screen for small-molecule binders of PAX3::FOXO1 protein, recombinant PAX3::FOXO1 was expressed in bacteria, and the protein was purified via column chromatography as described in Methods above. Recombinant PAX3::FOXO1 protein was purified to >90% purity at 50 μg/ml concentration (FIG. 1, Panel A). The identity of the recombinant PAX3::FOXO1 protein was confirmed by Western blot analysis using an anti-PAX3 antibody (FIG. 1, Panel B). DNA binding specificity of the purified protein was confirmed by comparing its binding with oligonucleotides specific for PAX3::FOXO1 or EWS::FLI1 using SPR. Purified PAX3::FOXO1 and EWS::FLI1 proteins were immobilized on neighboring flow cells of a CM5 chip by amine coupling and fusion protein-specific DNA oligonucleotides were injected over both protein-coated surfaces at a range of concentrations while the interactions were measured in real time. PAX3::FOXO1 protein bound to a PAX3::FOXO1-specific oligonucleotide but not an EWS::FLI1-specific one, while the reverse was true of EWS::FLI1 protein (FIG. 1, Panel C). The kinetics parameters of the purified recombinant PAX3::FOXO1 protein binding to its specific double-stranded DNA oligonucleotide were derived. A representative result is provided in FIG. 1, Panel D. The experiment was repeated six times, yielding an average K_{D} value of 6.3 nmol/L (SE 2.3 nmol/L). Therefore, it was concluded that the recombinant protein had a properly folded DNA binding domains and was deemed to be suitable for small molecule screening experiments.

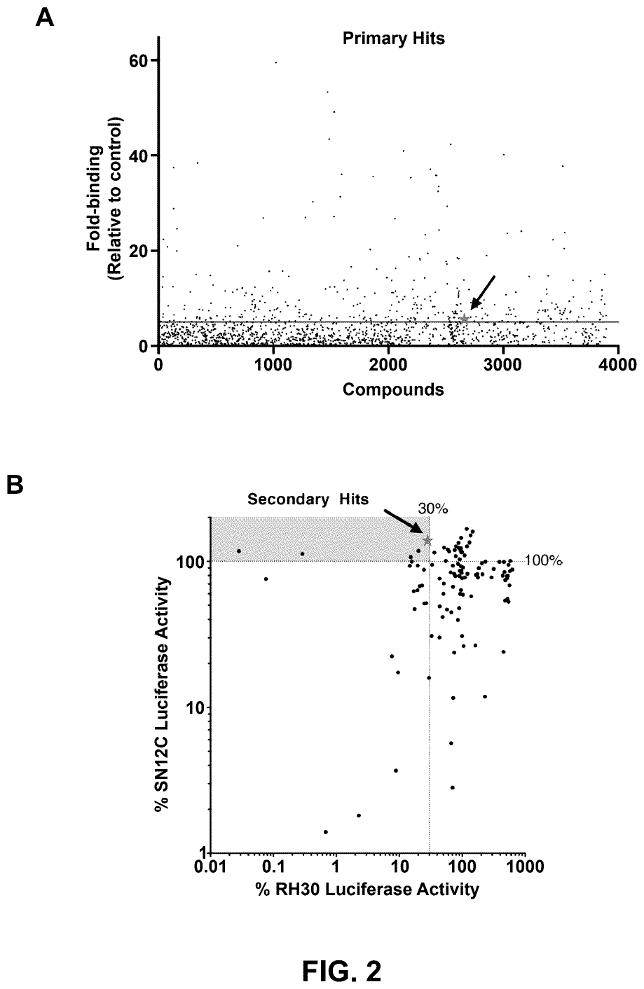

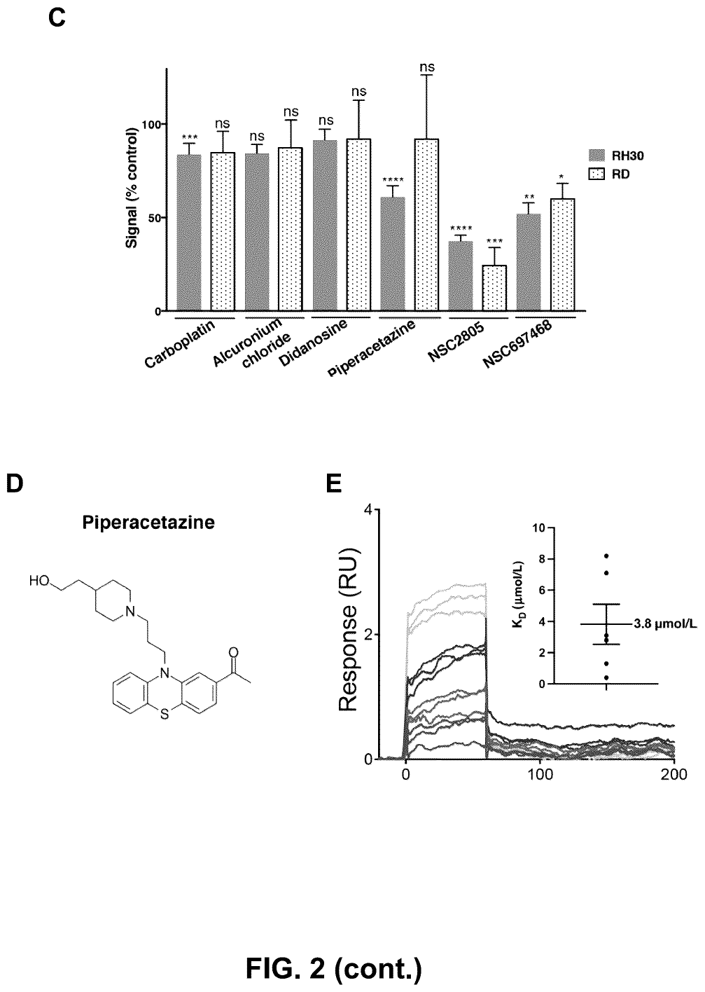

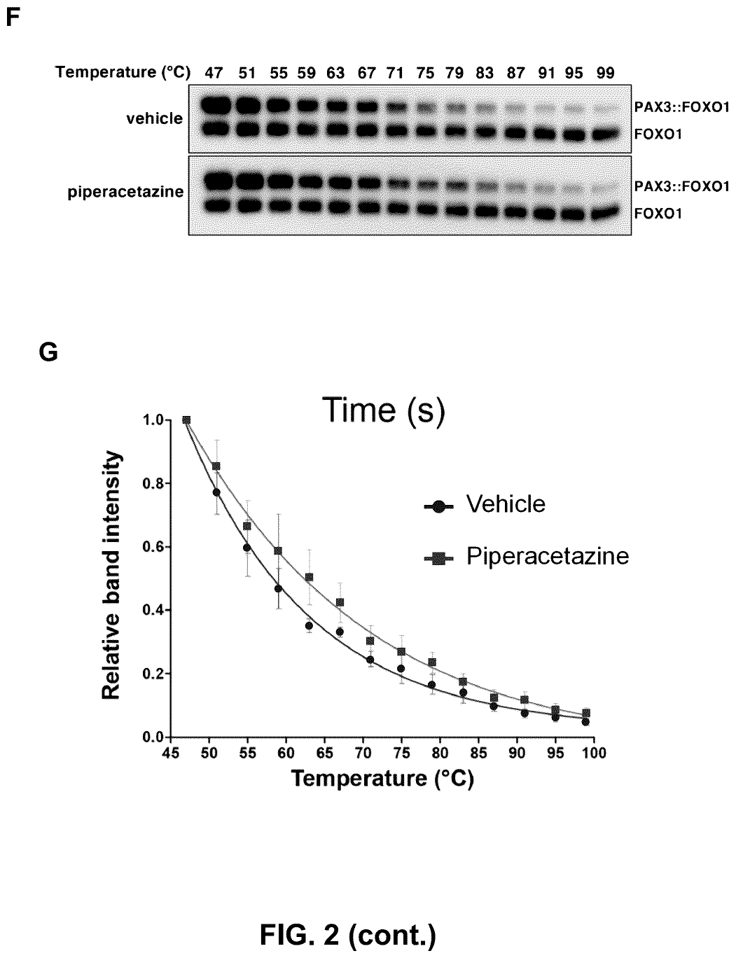

SPR was used for screening two small molecule libraries in a Biacore 4000 instrument. A total of 3,894 compounds covering a wide spectrum of structure classes from the Prestwick Chemical Company and the Developmental Therapeutics Program of the National Cancer Institute (diversity set, mechanistic set, natural products set) were screened. The binding of each compound to recombinant PAX3::FOXO1 protein was compared with the binding to another intrinsically disordered recombinant protein with a carboxy terminal His tag, CD99, which was used as a negative control. On the basis of a stringent hit selection criteria as explained in the Methods above, 119 compounds capable of binding to PAX3::FOXO1 were selected, which were advanced to a secondary screening assay based on PAX3::FOXO1 activity (FIG. 2, Panel A; Table 1).

The secondary screen that evaluated inhibition of PAX3::FOXO1 transcription initiation used the FP-RMS cell line RH30. PAX3::FOXO1 activates 6XPRS promoter luciferase construct allowing for easy quantification of its function. As a negative control, the renal cell carcinoma cell line SN12C that was stably transfected with a human PGK promoter luciferase construct was used. Both cell lines were treated with each one of the 119 primary hits at a 10 μmol/L concentration for 24 hours and luciferase activity was then measured. Compounds passed the secondary screen if they were capable of inhibiting PAX3::FOXO1-responsive luciferase activity more than 70% of the control without inhibiting PGK-responsive luciferase. Six secondary hits were identified as potential inhibitors of PAX3::FOXO1 (FIG. 2, Panel B; Table 2). For further validation of PAX3::FOXO1 inhibitory activity, the six secondary hits were evaluated with a different PAX3::FOXO1-responsive promoter. The FP-RMS cell line RH30 and FN-RMS cell line RD were transfected with the PAX3::FOXO1-responsive PDGFRA luciferase reporter. Cells were treated with 10 μmol/L concentration of compounds for 48 hours and luciferase activity was measured (FIG. 2, Panel C). In this experiment, piperacetazine showed the most significant PAX3::FOXO1-specific response. Alcuronium chloride and didanosine did not inhibit the PDGFRA luciferase reporter. Carboplatin inhibition was less significant than inhibition by piperacetazine. Both NSC2805 and NSC697468 inhibited the PDGFRA luciferase reporter in FP-RMS cell line RH30, but they also inhibited the same reporter in FN-RMS cell line RD at a similar level and were therefore considered nonspecific. Therefore, piperacetazine was selected for further evaluation (FIG. 2, Panel D). To calculate the binding affinity of piperacetazine to PAX3::FOXO1 protein, SPR experiments were performed using a Biacore T200 instrument. Piperacetazine was injected at multiple different concentrations over the PAX3::FOXO1 protein bound to a CM5 chip surface. An average binding affinity (K_{D}) of 3.8 μmol/L (1.2 μmol/L S.E.) was detected between the recombinant PAX3::FOXO1 protein and piperacetazine after six independent experiments. A representative binding sensorgram is presented in FIG. 2, Panel E.

To confirm that piperacctazine was capable of binding to endogenous PAX3::FOXO1 protein in RMS cells, a CETSA was performed. RH30 cell lysates were incubated with 30 μmol/L piperacetazine to saturate binding sites, or DMSO for 30 minutes and then exposed to an increasing range of temperatures for three minutes. Proteins that aggregated because of heat were separated by centrifugation, and the proteins remaining in solution were then analyzed by Western blot analysis. The CETSA allowed for the determination that piperacetazine bound and stabilized endogenous PAX3::FOXO1, which remained in solution at higher temperatures (FIG. 2, Panel F). The experiment was repeated three times and band intensities for each temperature were quantified by densitometric analysis (FIG. 2, Panel G). The shift to the right was observed for PAX3::FOXO1 protein (top band in each blot, FIG. 2, Panel F) but not the wild-type FOXO1 (bottom band in each blot, FIG. 2, Panel F). This finding suggests that piperacetazine is capable of binding to endogenous PAX3::FOXO1 protein in the presence of its natural binding partners in RMS cells.

### Piperacetazine Inhibits the Transcriptional Activity of Endogenous PAX3::FOXO1 on Luciferase Reporters in Multiple Fusion-Positive RMS Cells.

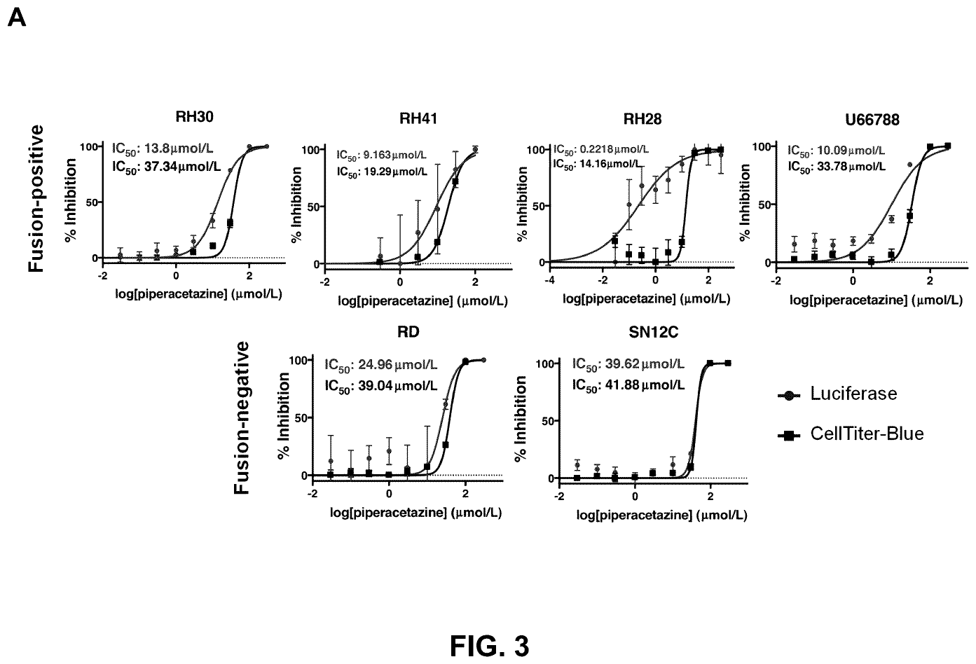

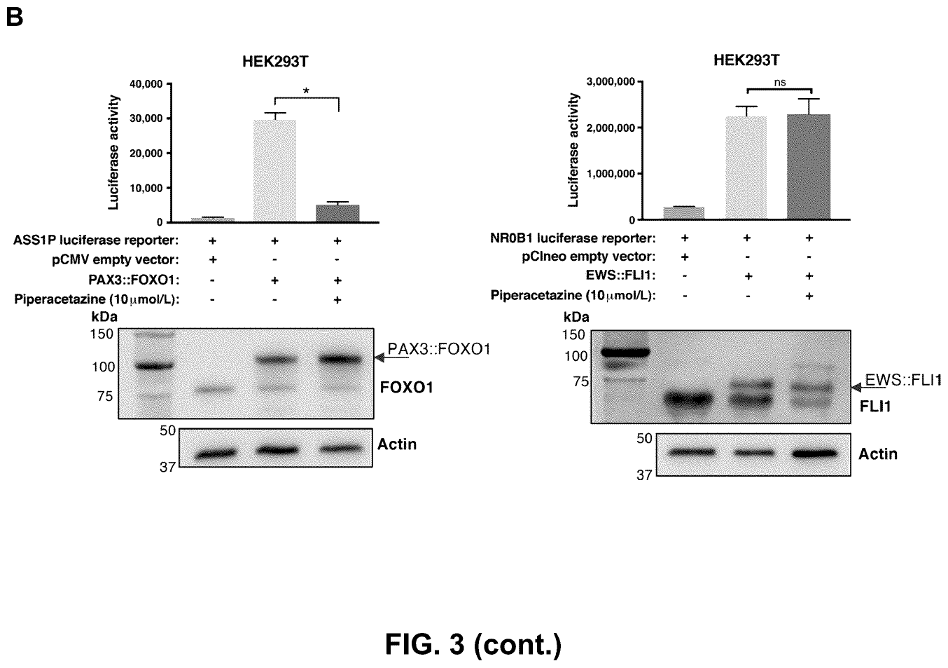

To confirm that piperacetazine specifically inhibits endogenous PAX3::FOXO1's transcriptional activity in FP-RMS cell lines, the pGL3-PDGFRA reporter construct was used. This reporter construct was transfected into four FP-RMS cell lines, (RH30, RH28/L-PAM, RH41, U66788) and one FN-RMS (RD) cell line. The non-RMS cell line SN12C with the PGK promoter luciferase construct was also included as an additional negative control. The cells were treated with a range of piperacetazine concentrations, and after 48 hours, relative luciferase activity was measured by luminometer, while cell viability was measured by fluorescence using the CellTiter Blue assay (FIG. 3, Panel A). In all four FP-RMS cell lines, luciferase activity was inhibited at a lower dose of piperacetazine than what was required to kill the cells, indicating a PAX3::FOXO1 specific activity. In contrast, in cells lacking PAX3::FOXO1, piperacetazine inhibited luciferase activity at doses that also induced cell death, indicating a nonspecific toxicity. This was observed as overlapping luciferase and cell viability curves. The consistent and reproducible shift of luciferase curves compared to cell viability curves in all four FP-RMS cell lines supports the hypothesis that piperacetazine inhibits transcriptional activity of PAX3::FOXO1 protein.

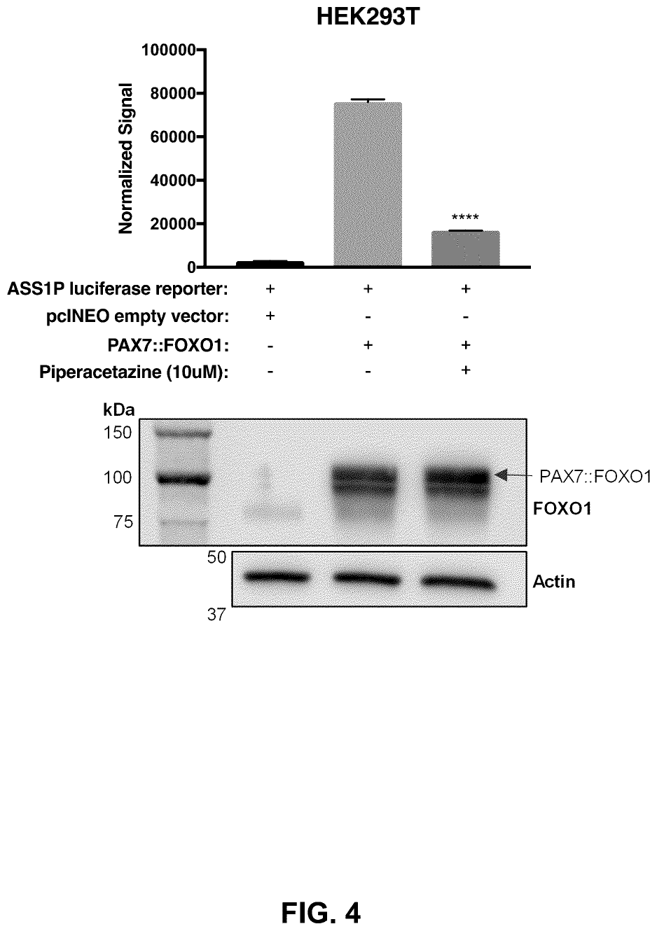

Further validation of piperacetazine as a specific PAX3::FOXO1 inhibitor occurred using a PAX3::FOXO1-responsive ASS1P luciferase reporter. An empty vector or PAX3::FOXO1 construct was cotransfected with the ASS1P luciferase reporter into HEK293T cells. The luciferase activity was evaluated after 48 hours of DMSO or piperacetazine treatment (FIG. 3, Panel B, left). PAX3::FOXO1 induced a high luciferase signal that decreased significantly with piperacetazine treatment. This response to piperacetazine treatment was not seen when the HEK293T cells were transfected with a construct for a different fusion protein EWS::FLI1 and an EWS::FLI1-responsive NR0B1 luciferase reporter as a negative control (FIG. 3, Panel B, right). These results further support the hypothesis that the transcriptional activity of PAX3::FOXO1 is selectively inhibited by piperacetazine. The inhibitory effect of piperacetazine on the PAX7::FOXO1 fusion protein was also evaluated in the same experimental system (FIG. 4). Expression of PAX7::FOXO1 activated the ASS1P luciferase reporter in HEK293T cells, which was significantly reduced by 48 hours of piperacetazine treatment, demonstrating that piperacetazine may also inhibit the transcriptional activity of PAX7::FOXO1.

TABLE 1
Compounds capable of binding to PAX3::FOXO1, as demonstrated in Example 1.

| | | Molecular |
|---|---|---|
| Common Name | IUPAC Name | Formula |
| carboplatin | cis-Diammine(1,1- | C₆H₁₄N₂O₄Pt |
| (NSC241240) | cyclobutanedicarboxylato)platinum(II) | |
| dasatinib | N-(2-chloro-6-methylphenyl)-2-[[6-[4-(2- | C₂₂H₂₆ClN₇O₂S |
| (NSC732517) | hydroxyethyl)piperazin-1-yl]-2-methylpyrimidin-4- | |
| | yl]amino]-1,3-thiazole-5-carboxamide | |
| lenvatinib | 4-[3-chloro-4-(cyclopropylcarbamoylamino)phenoxy]- | C₂₁H₁₉ClN₄O₄ |
| (NSC755980) | 7-methoxyquinoline-6-carboxamide | |
| rucaparib phosphate | 6-fluoro-2-[4-(methylaminomethyl)phenyl]-3,10- | C₁₉H₂₁FN₃O₅P |
| (NSC756644) | diazatricyclo[6.4.1.04, 13]trideca-1,4,6,8(13)-tetraen-9- | |
| | one; phosphoric acid | |
| idarubicin | (7S,9S)-9-acetyl-7-[(2R,4S,5S,6S)-4-amino-5-hydroxy- | C₂₆H₂₈ClNO₉ |
| hydrochloride | 6-methyloxan-2-yl]oxy-6,9,11-trihydroxy-8,10-dihydro- | |
| (NSC256439) | 7H-tetracene-5,12-dione; hydrochloride | |
| irinotecan | [(19S)-10,19-diethyl-19-hydroxy-14,18-dioxo-17-oxa- | C₃₃H₃₉ClN₄O₆ |
| hydrochloride | 3,13- | |
| (NSC616348) | diazapentacyclo[11.8.0.02,11.04,9.015,20]henicosa- | |
| | 1(21),2,4(9),5,7,10,15(20)-heptaen-7-yl] 4-piperidin-1- | |
| | ylpiperidine-1-carboxylate; hydrochloride | |
| NSC34210 | N-butan-2-yl-3-methylbutanamide | C₉H₁₉NO |
| tris-p-tolylsulfonium | tris(4-methylphenyl)sulfanium | C₂₁H₂₁S+ |
| (NSC157930) | | |
| NSC60530 | triazolo[5,1-f][1,2,4]triazine-4,6-diamine | C₄H₅N₇ |
| sulisobenzonum | 2-(4-hydroxy-2-oxo-3H-1,3-thiazol-5-yl)acetic acid | C₅H₅NO₄S |
| (NSC60548) | | |
| NSC63314 | 5-ethyl-2-[(E)-2-methylpent-1-enyl]pyridine | C₁₃H₁₉N |
| 2-benzoylpyrrole | phenyl(1H-pyrrol-2-yl)methanone | C₁₁H₉NO |
| (NSC75585) | | |
| 1-phenyl-5- | 2-phenylpyrazol-3-amine | C₉H₉N₃ |
| aminopyrazole | | |
| (NSC75786) | | |
| NSC80807 | (Z)-4-oxo-4-(1,3-thiazol-2-ylamino)but-2-enoic acid | C₇H₆N₂O₃S |
| NSC83345 | 4-phenyl-4,5,6,7-tetrahydro-1H-imidazo[4,5-c]pyridine | C₁₂H₁₃N₃ |
| NSC84200 | 3-[(dimethylamino)methyl]bicyclo[2.2.1]heptan-2-ol | C₁₀H₁₉NO |
| NSC299187 | 1-hydroxy-4-[2-(2- | C₁₈H₁₈N₂O₄ |
| | hydroxyethylamino)ethylamino]anthracene-9,10-dione | |
| NSC109813 | 2,5,8-trimethyl-1H-quinolin-4-one | C₁₂H₁₃NO |
| NSC2805 | 2-(2,5-dihydroxy-4-methylphenyl)-5-methylbenzene- | C₁₄H₁₄O₄ |
| | 1,4-diol | |
| NSC3001 | 2-[1-(carboxymethyl)-3-methylcyclohexyl]acetic acid | C₁₁H₁₈O₄ |
| NSC8179 | (5-phenyl-1H-1,2,4-triazol-3-yl)urea | C₉H₉N₅O |
| NSC8481 | 2-(4-tert-butylphenoxy)acetic acid | C₁₂H₁₆O₃ |
| NSC10416 | 9-Phenylcarbazole | C₁₈H₁₃N |
| quinacrine | 4-N-(6-chloro-2-methoxyacridin-9-yl)-1-N,1-N- | C₂₃H₃₂C₁₃N₃O |
| dihydrochloride | diethylpentane-1,4-diamine; dihydrochloride | |
| (NSC14229) | | |
| NSC513815 | 5-pyridin-3-yl-1,3,4-thiadiazol-2-amine | C₇H₆N₄S |
| NSC660300 | 1-(2-amino-4-nitrophenyl)piperidine-2,6-dione | C₁₁H₁₁N₃O₄ |
| NSC211336 | N-[3-(2-chloro-4-nitrophenoxy)propyl]acetamide | C₁₁H₁₃ClN₂O₄ |
| NSC305329 | 3-(benzimidazol-2-ylideneamino)-2H-isoindol-1-ol | C₁₅H₁₀N₄O |
| NSC623093 | 2,2,2-trifluoro-N-[2-[5-oxo-2,4- | C₂₅H₁₇F₃N₆O₂S₃ |
| | bis(phenylcarbamothioyl)-3-sulfanylidene-1,2,4-triazin- | |
| | 6-yl]phenyl]acetamide | |
| NSC27305 | 9-[6-(hydroxymethyl)-2,2-dimethyl-3a,4,6,6a- | C₁₃H₁₆N₄O₄S |
| | tetrahydrofuro[3,4-d][1,3]dioxol-4-yl]-3H-purine-6- | |
| | thione | |
| NSC629659 | 2-(1,3-benzothiazol-2-yl)-6-phenyl-4,5- | C₁₇H₁₃N₃OS |
| | dihydropyridazin-3-one | |
| NSC61642 | 6,7-bis(4-aminophenyl)pteridine-2,4-diamine | C₁₈H₁₆N₈ |
| boldine (NSC65689) | (6aS)-1,10-dimethoxy-6-methyl-5,6,6a,7-tetrahydro-4H- | C₁₉H₂₁NO₄ |
| | dibenzo[de, g]quinoline-2,9-diol | |
| NSC167410 | 2-(3,4-dihydroxyphenyl)-6,8-dihydroxy-3-(4,5,6- | C₂₁H₂₀O₁₁ |
| | trihydroxy-3-methyloxan-2-yl)oxychromen-4-one | |
| hydroberberubin | 16-methoxy-5,7-dioxa-13- | C₁₉H₁₉NO₄ |
| (NSC123389) | azapentacyclo[11.8.0.02,10.04,8.015,20]henicosa- | |
| | 2,4(8),9,15(20),16,18-hexaen-17-ol | |
| methergine | N-(1-hydroxybutan-2-yl)-7-methyl-6,6a,8,9-tetrahydro- | C₂₀H₂₅N₃O₂ |
| (NSC186067) | 4H-indolo[4,3-fg]quinoline-9-carboxamide | |
| NSC632536 | 4-N-phenyl-1-N-(2,2,6,6-tetramethylpiperidin-4- | C₂₁H₂₉N₃ |
| | yl)benzene-1,4-diamine | |
| NSC204232 | 1-N,3-N-bis(3-nitrophenyl)benzene-1,3-dicarboxamide | C₂₀H₁₄N₄O₆ |
| 9-aminocamptothecin | (19S)-8-amino-19-ethyl-19-hydroxy-17-oxa-3,13- | C₂₀H₁₇N₃O₄ |
| (NSC603071) | diazapentacyclo[11.8.0.02,11.04,9.015,20]henicosa- | |
| | 1(21),2,4,6,8,10,15(20)-heptaene-14,18-dione | |
| polyoxomolybdate | | HMo₁₂Na₂O₄₀P |
| phosphate complex, | | |
| disodium salt | | |
| (NSC622116) | | |
| quinic acid | (3S,5S)-1,3,4,5-tetrahydroxycyclohexane-1-carboxylic | C₇H₁₂O₆ |
| (NSC1115) | acid | |
| aristolochic acid | 8-methoxy-6-nitronaphtho[2,1-g][1,3]benzodioxole-5- | C₁₇H₁₁NO₇ |
| (NSC11926) | carboxylic acid | |
| xanthine | 3,7-dihydropurine-2,6-dione | C₅H₄N₄O₂ |
| (NSC14664) | | |
| deuteroporphyrin | 3-[18-(2-carboxyethyl)-3,8,13,17-tetramethyl-22,23- | C₃₀H₃₀N₄O₄ |
| (NSC18298) | dihydroporphyrin-2-yl]propanoic acid | |
| aspergillic acid | 6-butan-2-yl-1-hydroxy-3-(2-methylpropyl)pyrazin-2- | C₁₂H₂₀N₂O₂ |
| (NSC22939) | one | |
| (−)-cephaeline | (1R)-1-[[(2S,3R,11bS)-3-ethyl-9,10-dimethoxy- | C₂₈H₄₀C₁₂N₂O₄ |
| dihydrochloride | 2,3,4,6,7,11b-hexahydro-1H-benzo[a]quinolizin-2- | |
| (NSC32944) | yl]methyl]-7-methoxy-1,2,3,4-tetrahydroisoquinolin-6-ol; | |
| | dihydrochloride | |
| NSC96932 | (2Z)-3-ethyl-2-[(E)-3-(3-ethyl-1,3-benzothiazol-3-ium- | C₂₂H₂₃IN₂S₂ |
| | 2-yl)-2-methylprop-2-enylidene]-1,3- | |
| | benzothiazole; iodide | |
| isolupinine, (D), N- | (5-oxido-2,3,4,6,7,8,9,9a-octahydro-1H-quinolizin-5- | C₁₀H₁₉NO₂ |
| oxide | ium-4-yl)methanol | |
| (NSC34552) | | |
| 3,4- | 1-[(3,4-dimethoxyphenyl)methyl]-6,7-dimethoxy-3,4- | C₂₂H₂₅NO₈ |
| dihydropapaverine | dihydroisoquinoline; oxalic acid | |
| oxalate (NSC35550) | | |
| NSC56737 | N-[(E)-(3-methoxyphenyl)methylideneamino]-2-[1- | C₂₂H₂₆N₄O₄S |
| | [(2Z)-2-[(3-methoxyphenyl)methylidene]hydrazinyl]-1- | |
| | oxopropan-2-yl]sulfanylpropanamide | |
| pseudotropine | (1S,5R)-8-methyl-8-azabicyclo[3.2.1]octan-3-ol | C₈H₁₅NO |
| (NSC43871) | | |
| thapsine acetate | acetic acid; 5-[2-(dimethylamino)ethyl]-7,14-dimethoxy- | C₂₂H₂₃NO₈ |
| (NSC76022) | 2,9-dioxatetracyclo[6.6.2.04,16.011,15]hexadeca- | |
| | 1(14),4(16),5,7,11(15),12-hexaene-3,10-dione | |
| NSC169676 | 2-[2-[4-[3-[2-(trifluoromethyl)phenothiazin-10- | C₂₄H₃₄C₁₃F₃N₄OS |
| | yl]propyl]piperazin-1- | |
| | yl]ethylamino]ethanol; trihydrochloride | |
| echinatine | [(7S,8R)-7-hydroxy-5,6,7,8-tetrahydro-3H-pyrrolizin-1- | C₁₅H₂₅NO₅ |
| (NSC89937) | yl]methyl (2S)-2-hydroxy-2-[(1S)-1-hydroxyethyl]-3- | |
| | methylbutanoate | |
| NSC177365 | 2-[3-[[4-[(3-nitroacridin-9- | C₂₃H₂₃N₇O₄S |
| | yl)amino]phenyl]sulfamoyl]propyl]guanidine | |
| NSC316157 | 1,4-dihydroxy-2-[2-(2- | C₁₈H₁₈N₂O₅ |
| | hydroxyethylamino)ethylamino]anthracene-9,10-dione | |
| NSC335142 | 5,11-dimethyl-6H-pyrido[4,3-b]carbazole-1- | C₁₈H₁₆ClN₃₀ |
| | carboxamide; hydrochloride | |
| NSC624169 | 2-[[2-[(2- | C₁₂H₂₁ClN₂S₄ |
| | aminoethyldisulfanyl)methyl]phenyl]methyldisulfanyl]e | |
| | thanamine; hydrochloride | |
| NSC288010 | 1-(4-chlorophenyl)-N-[2-(diethylamino)ethyl]-7- | C₂₅H₃₀C₁₂N₄O₂ |
| | methoxy-4,5-dihydrobenzo[g]indazole-3- | |
| | carboxamide; hydrochloride | |
| kasuagamycin | 1-(4-chlorophenyl)-N-[2-(diethylamino)ethyl]-7- | C₁₄H₂₅N₃O₉ |
| (NSC100858) | methoxy-4,5-dihydrobenzo[g]indazole-3- | |
| | carboxamide; hydrochloride | |
| pinaverium bromide | 4-[(2-bromo-4,5-dimethoxyphenyl)methyl]-4-[2-[2-(6,6- | C₂₆H₄₁Br₂NO₄ |
| | dimethyl-4- | |
| | bicyclo[3.1.1]heptanyl)ethoxy]ethyl]morpholin-4-ium | |
| | bromide | |
| irinotecan | (S)-4,11-diethyl-3,4,12,14-tetrahydro-4-hydroxy-3,14- | C₃₃H₄₅ClN₄O₉ |
| hydrochloride | dioxo1H-pyrano[3′,4′:6,7]-indolizino[1,2-b]quinolin-9- | |
| trihydrate | yl-[1,4′bipiperidine]-1′-carboxylate hydrochloride | |
| | trihydrate | |
| pantoprazole sodium | 5-(difluoromethoxy)-2-[(3,4-dimethoxypyridin-2- | C₁₆H₁₄F₂N₃NaO₄S |
| | yl)methylsulfinyl]benzimidazol-1-ide sodium | |
| acetylsalicylsalicylic | 2-(2-acetyloxybenzoyl)oxybenzoic acid | C₁₆H₁₂O₆ |
| acid | | |
| R(−) apomorphine | (6aR)-6-Methyl-5,6,6a,7-tetrahydro-4H- | C₃₄H₃₈C₁₂N₂O₅ |
| hydrochloride | dibenzo[de, g]quinoline-10,11-diol hydrochloride | |
| hemihydrate | hydrate | |
| didanosine | 9-[(2R,5S)-5-(hydroxymethyl)oxolan-2-yl]-3H-purin-6- | C₁₀H₁₂N₄O₃ |
| | one | |
| erlotinib | N-(3-Ethynylphenyl)-6,7-bis(2-methoxyethoxy)-4- | C₂₂H₂₃N₃O₄ |
| | quinazolinamine | |
| hydroxyacrine | 9-amino-1,2,3,4-tetrahydroacridin-4-ol (Z)-but-2- | C₁₇H₁₈N₃O₅ |
| maleate (R,S) | enedioic acid | |
| levamisole | (6S)-6-phenyl-2,3,5,6-tetrahydroimidazo[2,1- | C₁₁H₁₃ClN₂S |
| hydrochloride | b][1,3]thiazole hydrochloride | |
| meclofenamic acid | 2-[(2,6-Dichloro-3-methylphenyl)amino]benzoic acid | C₁₄H₁₂Cl₂NNaO₃ |
| sodium salt | sodium salt | |
| monohydrate | | |
| hycanthone | 1-(2-Diethylaminoethylamino)-4-(hydroxymethyl)-9- | C₂₀H₂₄N₂O₂S |
| | thioxanthenone | |
| methylergometrine | (6aR,9R)-N-[(2S)-1-hydroxybutan-2-yl]-7-methyl- | C₂₄H₂₉N₃O₆ |
| maleate | 6,6a,8,9-tetrahydro-4H-indolo[4,3-fg]quinoline-9- | |
| | carboxamide (Z)-but-2-enedioate | |
| amikacin hydrate | (2S)-4-Amino-N-{(1R,2S,3S,4R,5S)-5-amino-2-[(3- | C₂₂H₄₇N₅O₁₅ |
| | amino-3-deoxy-alpha-D-glucopyranosyl)oxy]-4-[(6- | |
| | amino-6-deoxy-alpha-D-glucopyranosyl)oxy]-3- | |
| | hydroxycyclohexyl}-2-hydroxybutanamide | |
| prochlorperazine | 2-chloro-10-[3-(4-methylpiperazin-1- | C₂₈H₃₂ClN₃O₈S |
| dimaleate | yl)propyl]phenothiazine (Z)-but-2-enedioic acid | |
| zuclopenthixol | 2-[4-[(3Z)-3-(2-chlorothioxanthen-9- | C₂₂H₂₇Cl₃N₃O₈S |
| dihydrochloride | ylidene)propyl]piperazin-1-yl]ethanol dihydrochloride | |
| sertraline | (1S,4S)-4-(3,4-dichlorophenyl)-N-methyl-1,2,3,4- | C₁₇H₁₇Cl₃N |
| | tetrahydronaphthalen-1-amine | |
| alcuronium chloride | 4,4′-Didemethyl-4,4′-di-propenyltoxiferin-1-dichloride | C₄₄H₅₀Cl₃N₄O₂ |
| sulfasalazine | 2-hydroxy-5-[(E)-2-{4-[(pyridin-2- | C₁₈H₁₄N₄O₅S |
| | yl)sulfamoyl]phenyl}diazen-1-yl]benzoic acid | |
| tobramycin | (2S,3R,4S,5S,6R)-4-amino-2-[(1S,2S,3R,4S,6R)-4,6- | C₁₈H₃₇N₅O₉ |
| | diamino-3-[(2R,3R,5S,6R)-3-amino-6-(aminomethyl)-5- | |
| | hydroxyoxan-2-yl]oxy-2-hydroxycyclohexyl]oxy-6- | |
| | (hydroxymethyl)oxane-3,5-diol | |
| paroxetine | (3S,4R)-3-(1,3-benzodioxol-5-yloxymethyl)-4-(4- | C₁₉H₂₁ClFNO₉ |
| hydrochloride | fluorophenyl)piperidine hydrochloride | |
| liothyronine | (2S)-2-amino-3-[4-(4-hydroxy-3-iodophenoxy)-3,5- | C₁₅H₁₂I₃NO₄ |
| | diiodophenyl]propanoic acid | |
| demecarium bromide | trimethyl-[3-[methyl-[10-[methyl-[3- | C₃₂H₅₂Br₂N₄O₄ |
| | (trimethylazaniumyl)phenoxy]carbonylamino]decyl]car | |
| | bamoylJoxyphenyl]azanium dibromide | |
| bephenium | benzyl-dimethyl-(2-phenoxyethyl)azanium 3- | C₂₈H₂₉NO₄ |
| hydroxynaphthoate | carboxynaphthalen-2-olate | |
| benserazide | 3-[6-(3-carboxy-2,4,6-triiodoanilino)-6- | C₂₀H₁₄I₆N₂O₆ |
| hydrochloride | oxohexanoyl]amino]-2,4,6-triiodobenzoic acid | |
| salmeterol | 2-(hydroxymethyl)-4-[1-hydroxy-2-[6-(4- | C₂₅H₃₇NO₄ |
| | phenylbutoxy)hexylamino]ethyl]phenol | |
| prazosin | [4-(4-amino-6,7-dimethoxyquinazolin-2-yl)piperazin-1- | C₁₉H₂₁ClN₅O₄ |
| hydrochloride | yl]-(furan-2-yl)methanone hydrochloride | |
| stavudine | 1-[(2R,5S)-5-(hydroxymethyl)-2,5-dihydrofuran-2-yl]- | C₁₀H₁₂N₂O₄ |
| | 5-methylpyrimidine-2,4-dione | |
| megestrol acetate | 17-(acetyloxy)-6-methyl-pregna-4,6-diene-3,20-dione | C₂₄H₃₂O₄ |
| imatinib | 4-[(4-methylpiperazin-1-yl)methyl]-N-[4-methyl-3-[(4- | C₂₉H₃₁N₇O |
| | pyridin-3-ylpyrimidin-2-yl)amino]phenyl]benzamide | |
| aclacinomycin A | methyl (1R,2R,4S)-4-[(2R,4S,5S,6S)-4-(dimethylamino)- | C₄₂H₅₃NO₁₅ |
| (NSC208734) | 5-[(2S,4S,5S,6S)-4-hydroxy-6-methyl-5-[(2R,6S)-6- | |
| | methyl-5-oxooxan-2-yl]oxyoxan-2-yl]oxy-6- | |
| | methyloxan-2-yl]oxy-2-ethyl-2,5,7-trihydroxy-6,11- | |
| | dioxo-3,4-dihydro-1H-tetracene-1-carboxylate | |
| epirubicin | (7S,9S)-7-[(2R,4S,5R,6S)-4-amino-5-hydroxy-6- | C₂₇H₃₀ClNO₁₁ |
| hydrochloride | methyloxan-2-yl]oxy-6,9,11-trihydroxy-9-(2- | |
| (NSC256942) | hydroxyacetyl)-4-methoxy-8,10-dihydro-7H-tetracene- | |
| | 5,12-dione; hydrochloride | |
| NSC332294 | (6aR,9R,10aR)-7,9-dimethyl-4-propyl-6,6a,8,9,10,10a- | C₂₃H₃₂N₂O₆ |
| | hexahydroindolo[4,3-fg]quinoline; (2R,3R)-2,3- | |
| | dihydroxybutanedioic acid | |
| albacarcin V | (6aR,9R,10aR)-7,9-dimethyl-4-propyl-6,6a,8,9,10,10a- | C₂₈H₂₈O₉ |
| (NSC354844) | hexahydroindolo[4,3-fg]quinoline; (2R,3R)-2,3- | |
| | dihydroxybutanedioic acid | |
| rebeccamycin | 5,21-dichloro-3-[(2R,3R,4R,5S,6R)-3,4-dihydroxy-6- | C₂₇H₂₁C₁₂N₃O₇ |
| (NSC359079) | (hydroxymethyl)-5-methoxyoxan-2-yl]-3,13,23- | |
| | triazahexacyclo[14.7.0.02,10.04,9.011,15.017,22]tricosa- | |
| | 1,4(9),5,7,10,15,17(22), 18,20-nonaene-12,14-dione | |
| imerubrine | 5,14,15,16-tetramethoxy-10- | C₂₀H₁₇NO₅ |
| (NSC785144) | azatetracyclo[7.7.1.02,8.013,17]heptadeca- | |
| | 1(16),2,4,7,9,11,13(17), 14-octaen-6-one | |
| homomoschatoline | 14,15,16-trimethoxy-10- | C₁₉H₁₅NO₄ |
| (NSC785149) | azatetracyclo[7.7.1.02,7.013,17]heptadeca- | |
| | 1(16),2,4,6,9,11,13(17),14-octaen-8-on | |
| dehydroanonaine | 3,5-dioxa-11- | C17H13NO2 |
| (NSC785156) | azapentacyclo[10.7.1.02,6.08,20.014,19]icosa- | |
| | 1(20),2(6),7,12,14,16,18-heptaene | |
| isoboldine | (6aS)-2,10-dimethoxy-6-methyl-5,6,6a,7-tetrahydro-4H- | C₁₉H₂₁NO₄ |
| (NSC785158) | dibenzo[de, g]quinoline-1,9-diol | |
| nandigerine | (12S)-18-methoxy-3,5-dioxa-11- | C₁₈H₁₇NO₄ |
| (NSC785160) | azapentacyclo[10.7.1.02,6.08,20.014,19]icosa- | |
| | 1(20),2(6),7,14(19), 15,17-hexaen-17-ol | |
| O- | (12S)-17,18-dimethoxy-11-methyl-3,5-dioxa-11- | C₂₀H₂₁NO₄ |
| methylbulbocapnine | azapentacyclo[10.7.1.02,6.08,20.014,19]icosa- | |
| (NSC785162) | 1(20),2(6),7,14(19), 15,17-hexaene | |
| O-methylsciadenine | (11R,26S)-4,5,19,20-tetramethoxy-10,25-dimethyl-2,17- | C₃₈H₄₂N₂O₆ |
| (NSC785174) | dioxa-10,25- | |
| | diazaheptacyclo[26.2.2.213,16.13,7.118,22.011,36.026, | |
| | 33]hexatriaconta- | |
| | 1(31),3(36),4,6,13,15,18(33),19,21,28(32),29,34- | |
| | dodecaene | |
| N-methyl | (12S)-18-methoxy-11-methyl-3,5-dioxa-11- | C₁₉H₁₉NO₄ |
| hernangerin | azapentacyclo[10.7.1.02,6.08,20.014,19]icosa- | |
| (NSC785182) | 1(20),2(6), 7,14(19), 15,17-hexaen-17-ol | |
| grisabine | (1R)-1-[[4-[5-[[(1S)-7-hydroxy-6-methoxy-2-methyl- | C₃₇H₄₂N₂O₆ |
| (NSC785183) | 3,4-dihydro-1H-isoquinolin-1-yl]methyl]-2- | |
| | methoxyphenoxy]phenyl]methyl]-6-methoxy-2-methyl- | |
| | 3,4-dihydro-1H-isoquinolin-7-ol | |
| 1,10-dihydroxy-2- | 2-methoxy-6-methyl-5,6,6a,7-tetrahydro-4H- | C₁₈H₁₉NO₃ |
| methoxyaporphine | dibenzo[de, g]quinoline-1,10-diol | |
| (NSC785186) | | |
| N- | (6aS)-1,2,10-trimethoxy-6-methyl-5,6,6a,7-tetrahydro- | C₂₀H₂₃NO₄ |
| methyllaurotetanine | 4H-dibenzo[de, g]quinolin-9-ol | |
| (NSC785189) | | |
| NSC697468 | 2-[2-[2-[5-acetamido-3-hydroxy-2-(hydroxymethyl)-6- | C₄₅H₅₈N₈O₁₂ |
| | phenylmethoxyoxan-4-yl]oxypropanoylamino]-3- | |
| | methylbutanoyl]amino]-N′-[4-[(1-nitroacridin-9- | |
| | yl)amino]butyl]pentanediamide | |
| NSC690634 | N,N-bis(1-nitroacridin-9-yl)propane-1,3-diamine | C₂₉H₂₂N₆O₄ |
| glycylaminophenylbe | 2-[(2-aminoacetyl)amino]-N-[[4-(5-bromopyrimidin-2- | C₂₁H₂₀BrClN₆O₄ |
| nzoylurea (HCl salt) | yl)oxy-3- | |
| (NSC654259) | methylphenyl]carbamoyl]benzamide; hydrochloride | |
| NSC635833 | methyl 4-(2,3-dimethylanilino)-2,4-dioxo-3-(3-oxo-4H- | C₂₁H₂₀N₂O₅S |
| | 1,4-benzothiazin-2-yl)butanoate | |
| NSC698031 | 1-[5-[(E)-2-[4-(dimethylamino)phenyl]ethenyl]-6- | C₂₄H₂₂N₆O₂S |
| | methyl-1,2,4-triazin-3-yl]-6-hydroxy-3-phenyl-2- | |
| | sulfanylidenepyrimidin-4-one | |
| etomidate | ′ethyl 3-(1-phenylethyl)imidazole-4-carboxylate | C₁₄H₁₆N₂O₂ |
| piperacetazine | 1-[10-[3-[4-(2-hydroxyethyl)piperidin-1- | C₂₄H₃₀N₂O₂S |
| | yl]propyl]phenothiazin-2-yl]ethanone | |
| metformin | 1-carbamimidamido-N,N-dimethylmethanimidamide | C₄H₁₂ClN₅ |
| hydrochloride | | |
| meticrane | 6-methylthiochromane-7-sulfonamide 1,1-dioxide | C₁₀H₁₃NO₄S₂ |
| mefenamic acid | 2-(2,3-dimethylphenyl)aminobenzoic acid | C₁₅H₁₅NO₂ |
| sulpiride | (±)-5-(aminosulfonyl)-N-[(1-ethylpyrrolidin-2- | C₁₅H₂₃N₃O₄S |
| | yl)methyl]-2-methoxybenzamide | |
| albendazole | methyl N-(6-propylsulfanyl-1H-benzimidazol-2- | C₁₂H₁₅N₃O₂S |
| | yl)carbamate | |
| quinacrine | 6-Chloro-9-(4-diethylamino-1-methylbutyl)amino-2- | C₂₃H₃₆C₁₃N₃O₃ |
| dihydrochloride | methoxyacridine dihydrochloride hydrate | |
| hydrate | | |

TABLE 2
Compounds capable of inhibiting PAX3::FOXO1-responsive
luciferase activity more than 70% of the control.

| | % Activity of | % Activity of | | Fold-Difference |
|---|---|---|---|---|
| | PAX3::FOXO1 | PGK (Relative | % Rₘₐₓ | PAX3::FOXO1 vs |
| Compound | (Relative to Control | to Control | PAX3::FOXO1 | Control Binding |
| Name | Treatment) | Treatment) | (SPR) | (SPR) |
| NSC697468 | 0.02823246 | 117.5048 | 137.50 | 17.20 |
| alcuronium | 15.77796 | 99.78218 | 55.45 | 13.87 |
| chloride | | | | |
| NSC2805 | 20.22917 | 117.8538 | 78.36 | 12.38 |
| didanosine | 15.17979 | 106.8316 | 62.86 | 6.70 |
| carboplatin | 0.287854 | 112.4189 | 180.54 | 6.19 |
| piperacetazine | 28.18571 | 136.0233 | 177.28 | 5.54 |

### Example 2

A study was conducted in which five PROTAC compounds, comprising NSC697468 as the PAX3::FOXO1 fusion protein binding moiety and pomalidomide as the E3 ubiquitin ligase binding moiety, were evaluated for their cytotoxicity in RH30 cells (human RMS cell line that expresses PAX3::FOXO1 fusion protein) and TC71 cells (Ewing sarcoma cell line that does not express PAX3::FOXO1 fusion protein), and for their efficacy in degrading PAX3::FOXO1 in RH30 cells in vitro. The PROTAC compounds are Compounds A, B, C, D, and G described in the Compounds of the Present Invention section, infra.

The processes used to synthesize the PROTAC compounds are shown in FIG. 5.

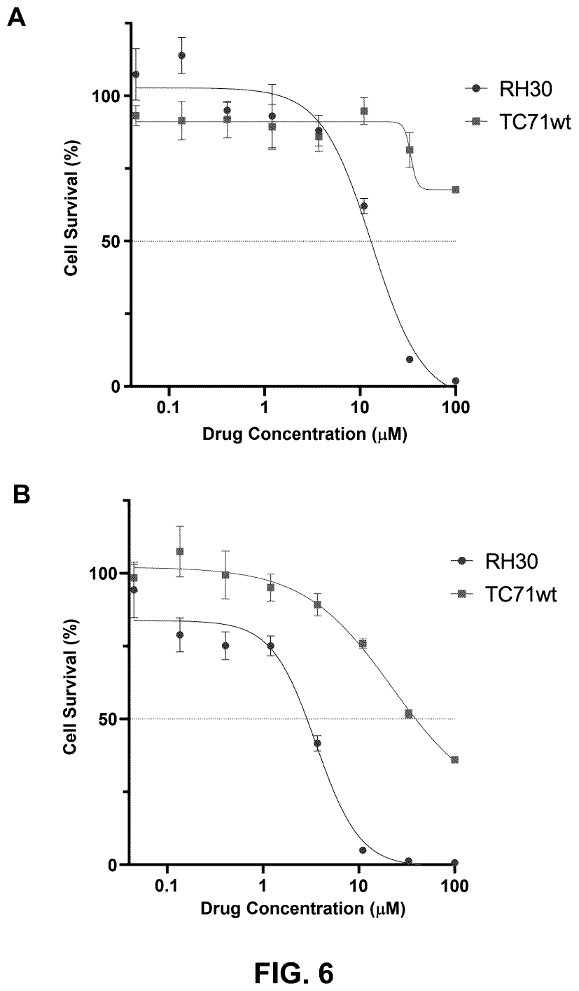

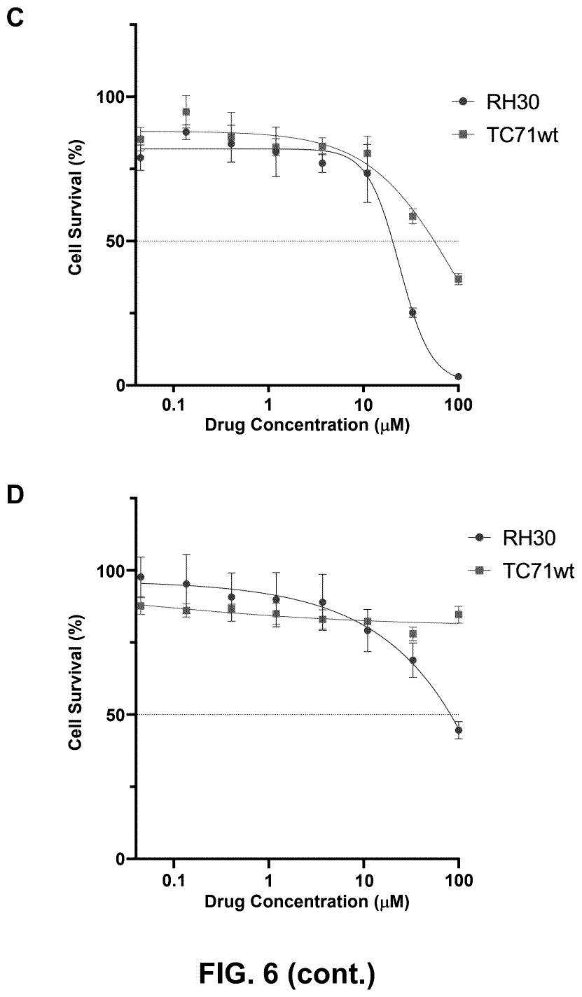

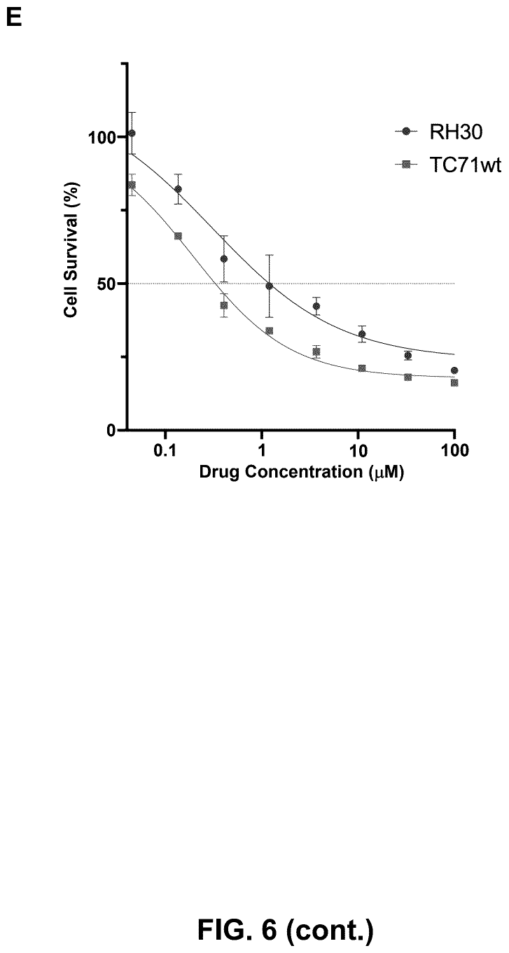

The RH30 and TC71 cells were exposed to the PROTAC compounds for 48 hours at concentrations ranging from 0.045 to 100 μM. The IC₅₀ was determined a cell viability assay. The results are shown in Table 3 and in FIG. 6, and indicate that, with one exception, the PROTAC compounds were generally more cytotoxic to the RH30 cells than to the TC71 cells. Also, for at least PROTAC Compounds B and C, a concentration range of about 1 to 50 μM may be cytotoxic to RH30 cells while being safe for TC71 cells.

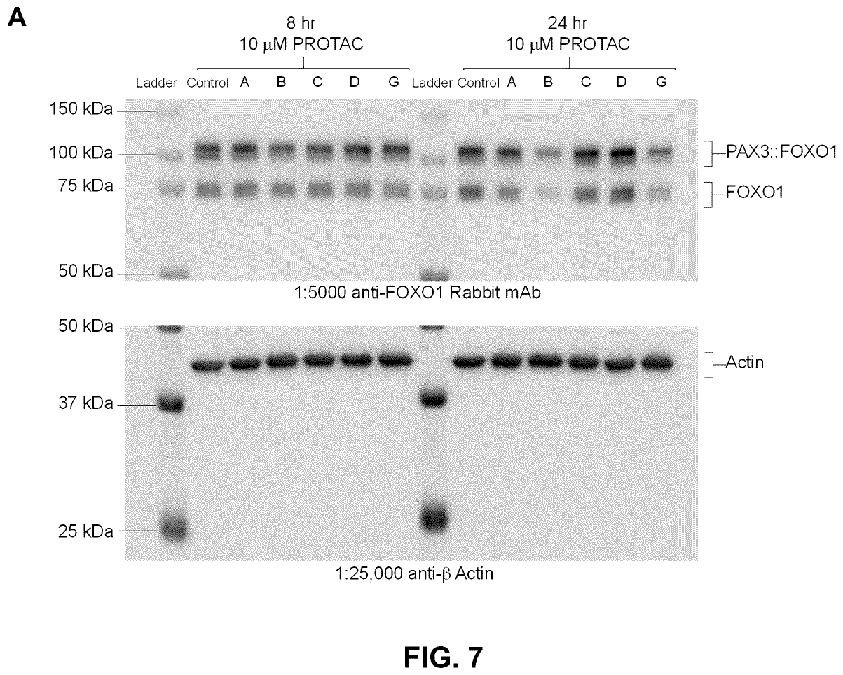

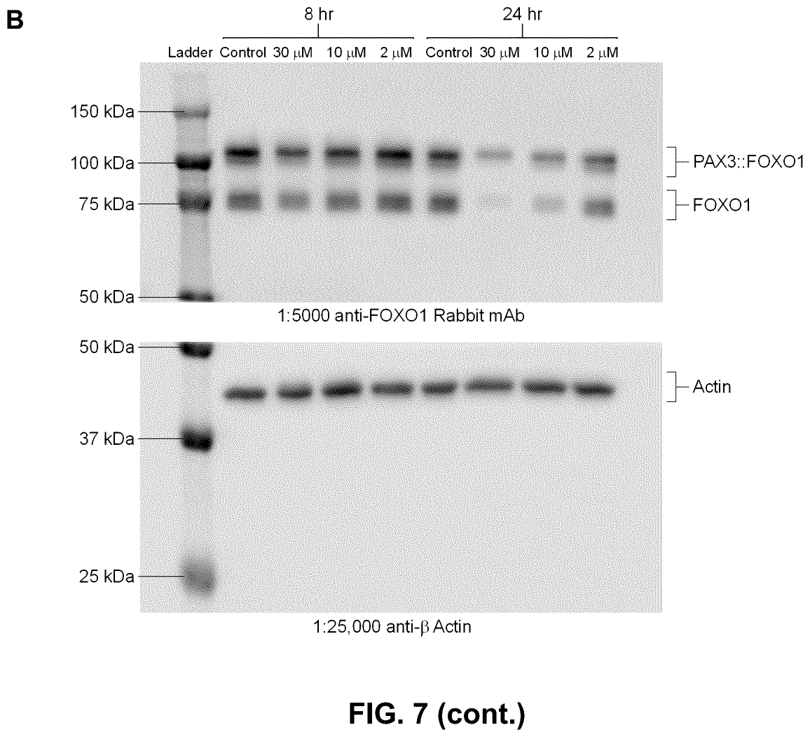

The efficacy of the PROTAC compounds on PAX3::FOXO1 degradation at 8 and 24 hours was evaluated using Western blot analysis. RH30 cells grown to 60 to 80% confluence in six-well plates and treated with the compounds in a RPMI medium with 0.2% dimethyl sulfoxide at a concentration of 10 μM. About 8.75 μg of the protein in 17.5 μL of lysate per sample was loaded in 15-well 10% acrylamide gels. Images of the Western blots are provided in FIG. 7, Panel A, which shows that PROTAC Compound B caused the greatest amount of degradation of both PAX3::FOXO1 and FOXO1 at 24 hours.

Compound B was further explored by monitoring differences in PAX3::FOXO1 degradation at 8 and 24 hrs from different concentrations of the compound. RH30 cells grown to 60 to 80% confluence in six-well plates and treated with the Compound B in a RPMI medium with 0.2% dimethyl sulfoxide at a concentrations 30, 10, and 2 μM. About 8.75 μg of the protein in 17.5 μL of lysate per sample was loaded in 15-well 10% acrylamide gels. Images of the Western blots are provided in FIG. 6, Panel B, which showed a dose dependency of Compound B on PAX3::FOXO1 and FOXO1 degradation at 24 hours.

TABLE 3
Cell survival IC₅₀ of RH30 and TC71 cells exposed
to PROTAC Compounds A, B, C, D, and G for 48 hours.

IC₅₀ Value (μM)

| PROTAC Compound | RH30 | TC71 |
|---|---|---|
| A | 13.31 | — |
| B | 3.57 | 22.14 |
| C | 23.99 | 96.2 |
| D | — | — |
| G | 0.31 | 0.19 |

### Example 3

A study was conducted in which two PROTAC compounds, comprising C6.18 as the PAX3::FOXO1 fusion protein binding moiety and pomalidomide as the E3 ubiquitin ligase binding moiety, were evaluated for their cytotoxicity in RH30 cells and RH41 cells (human ARMS cell line that expresses PAX3::FOXO1) following 48h treatment. The PROTAC compounds are Compounds 01-A1 and 01-E1 described in the Compounds of the Present Invention section, infra.

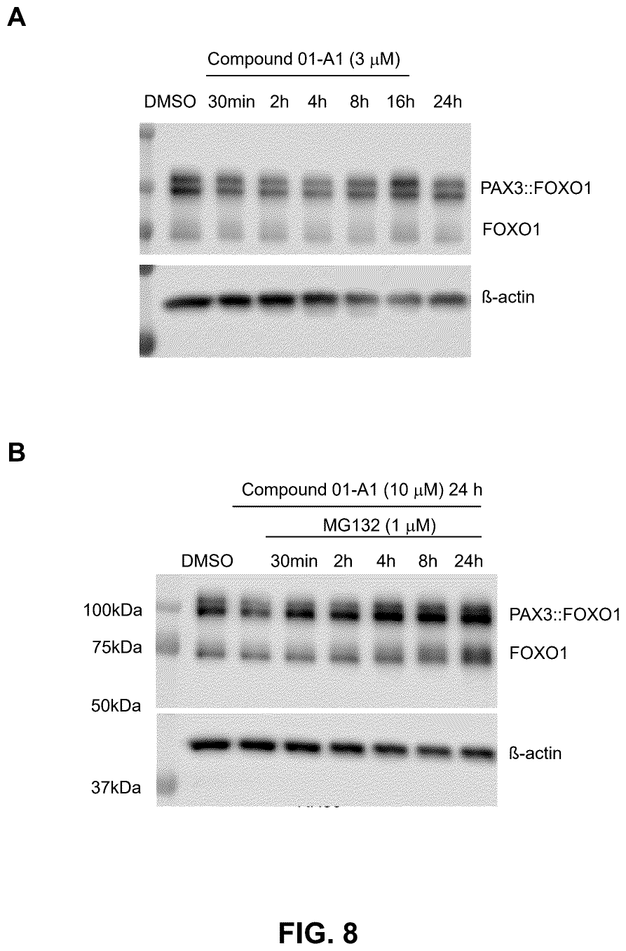

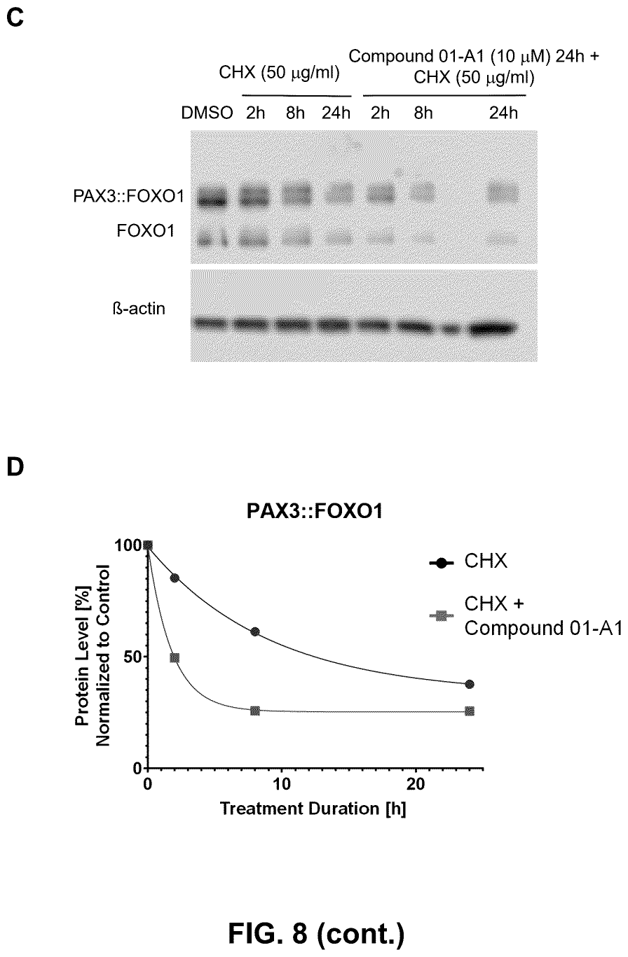

Compound 01-A1 was analyzed using three assays. In the first assay, RH30 cells were exposed to the PROTAC compound for up to 24 hours at concentration of 3 μM. The results of the Western blot analysis are shown in FIG. 8, Panel A. and indicate that the compound degrades PAX3::FOXO1. In the second assay, RH30 cells were treated with the PROTAC compound for 24 hours at concentration of 10 μM; and with MG132, which inhibits the proteasome and therefore stabilizes polyubiquitinated proteins, at a concentration of 1 μM for 30 minutes, 2 hours, 4 hours, 8 hours, and 24 hours. The results are shown in FIG. 8, Panel B, and confirm that, when treated with Compound 01-A1, PROTAC-mediated degradation of PAX3::FOXO1 is through the proteosome in the cell. The third assay was a cycloheximide (CHX) chase assay, in which RH30 cells were pre-treated with 50 μg/ml of CHX for 2 hours, 8 hours, or 24 hours, prior to treating with Compound 01-A1. CHX blocks new protein synthesis. Results are shown in FIG. 8, Panels C and D, which demonstrate that treatment with the compound resulted in degradation of both PAX3::FOXO1 and was not due to decreased protein synthesis. This is also reflected in the half-life measurement of PAX3::FOXO1 in CHX and Compound 01-A1 treated cells, which was 6.324 and 1.228 hours, respectively. This shows that when new protein synthesis was blocked and PAX3::FOXO1 decay was observed, Compound 01-A1 accelerates PAX3::FOXO1 degradation and reduces its half-life.

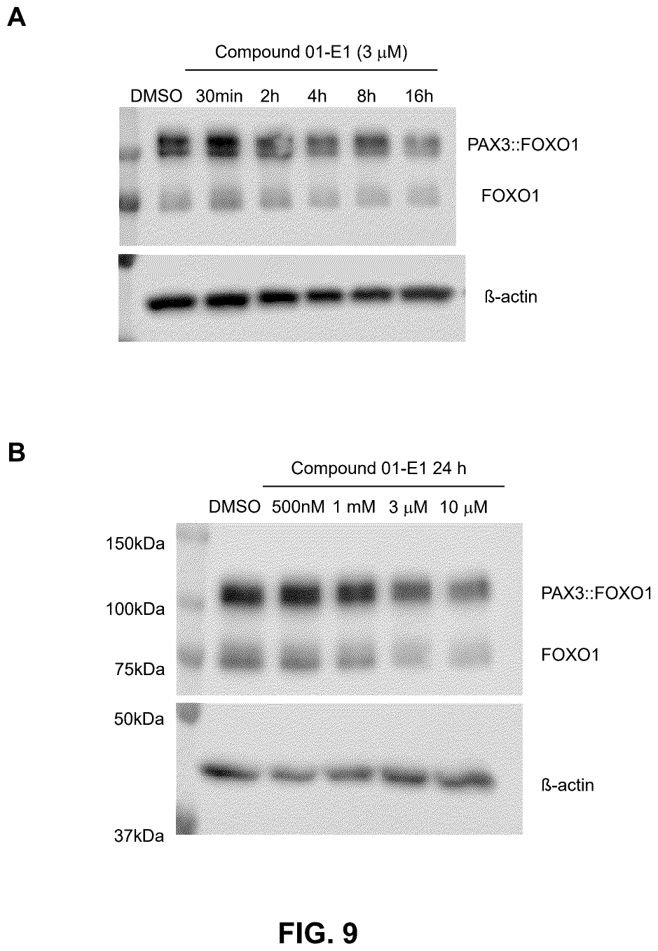

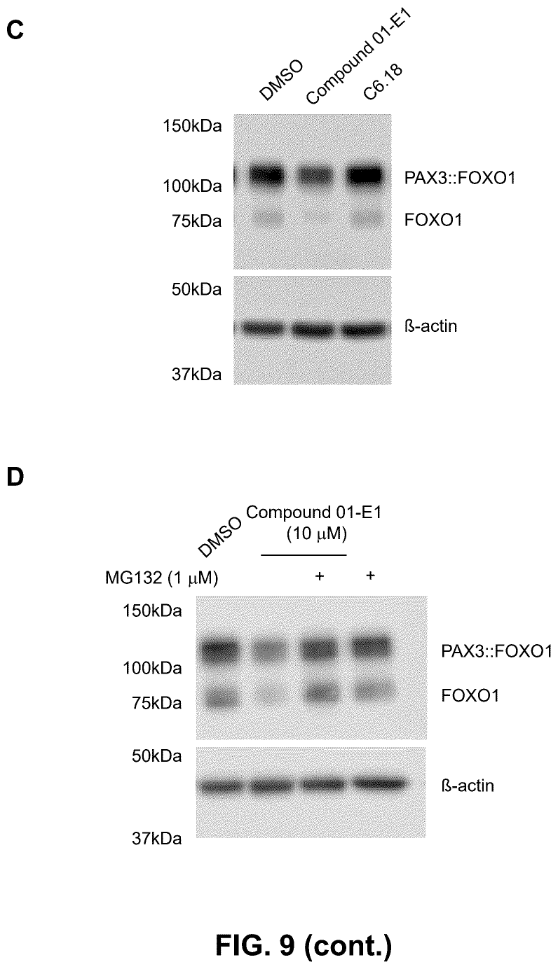

Compound 01-E1 was also analyzed using three assays. In the first assay, RH30 cells were exposed to the PROTAC compound for up to 24 hours at concentration of 3 μM, and for 24 hours at concentrations of 500 nM, 1 μM, 3 μM, and 10 μM. The results of the Western blot analysis are shown in FIG. 9, Panel A and B, and indicate that degradation of PAX3::FOXO1 is greater over time and at higher concentrations. In the second assay, RH41 cells were treated with the PROTAC compound and with the C6.18, the PAX3::FOXO1 fusion protein binding moiety only. The results of the Western blot analysis shown in FIG. 9, Panel C, indicate that exposure to C6.18 alone did not degrade the PAX3::FOXO1 fusion protein, and confirms the effect of the PROTAC structure. In the third assay, RH30 cells were treated with the PROTAC compound at concentration of 10 μM and with MG132 at a concentration of 1 μM. The results are shown in FIG. 9, Panel D, and confirm that, when treated with Compound 01-E1, degradation of PAX3::FOXO1 is through the proteosome in the cell.

### Example 4

A study was conducted in which two PROTAC compounds, comprising C6.12 or C6.15 as the PAX3::FOXO1 fusion protein binding moieties and pomalidomide as the E3 ubiquitin ligase binding moiety, were evaluated for their ability to degrade PAX3::FOXO1 protein in RH30 cells. The PROTAC compounds are Compounds 07-A and 07-E described in the Compounds of the Present Invention section, infra.

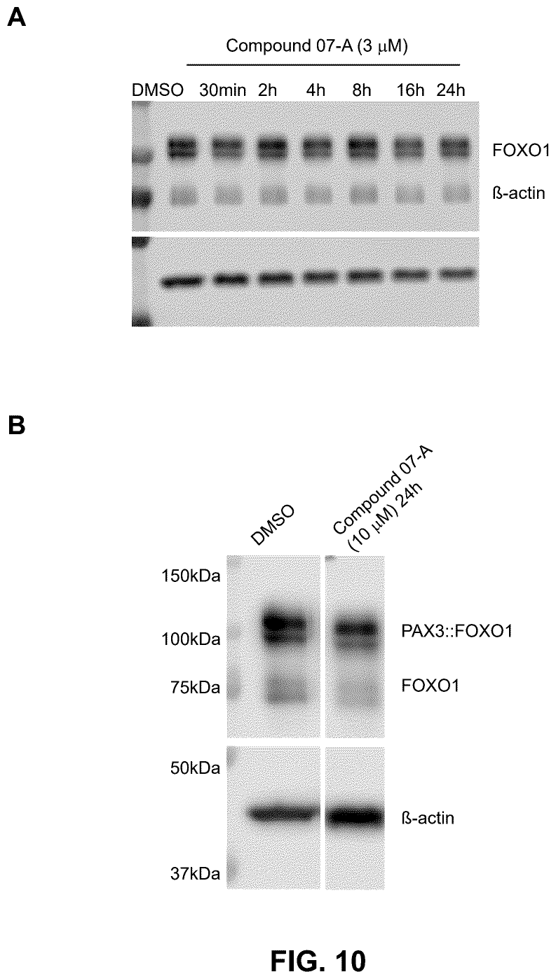

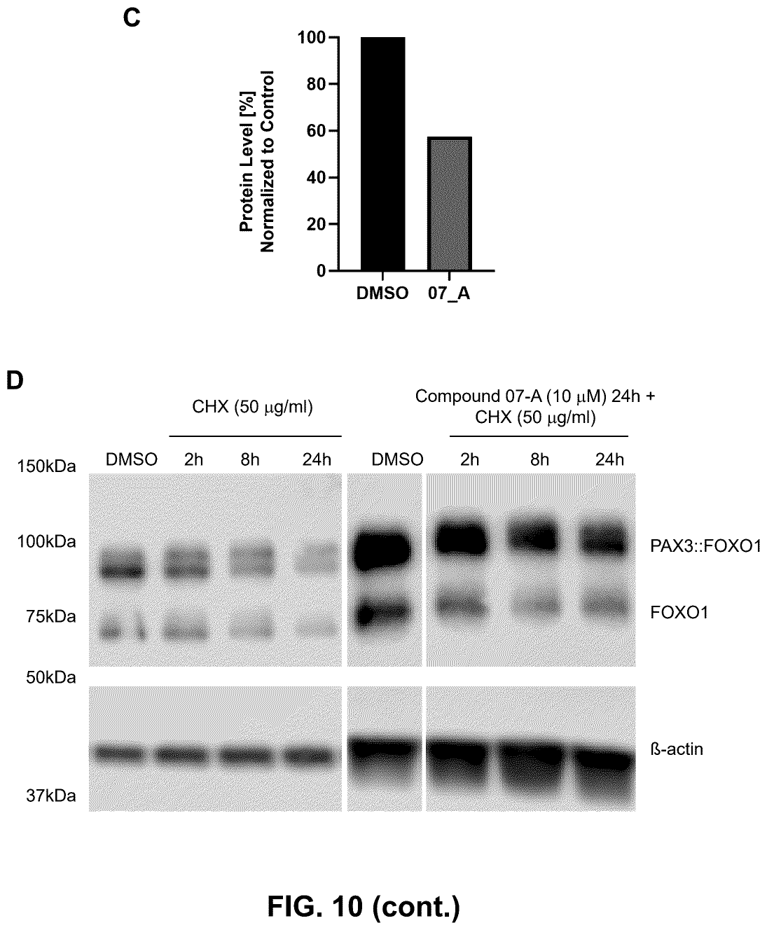

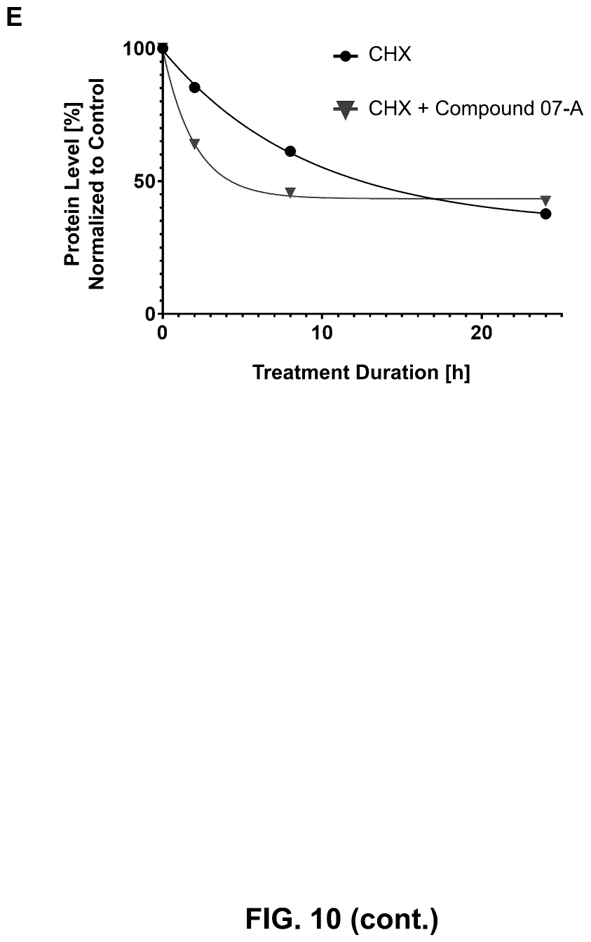

Compound 07-A was analyzed using two assays. In the first assay, RH30 cells were exposed to the PROTAC compound for up to 24 hours at concentration of 3 μM, and for 24 hours at a concentration of 10 μM. The results of the Western blot analysis are shown in FIG. 10, Panels A and B, and indicate that the compound degrades PAX3::FOXO1. Also, quantification of the PAX3::FOXO1 fusion protein in the cells show that protein level is about 40% less in cells treated by Compound 07-A as compared to DMSO control (see FIG. 10, Panel C). The second assay was a CHX chase assay, in which RH30 cells were pre-treated with 50 μg/ml of CHX for 2 hours, 8 hours, or 24 hours prior to treating with Compound 07-A at concentration of 10 μM. Results are shown in FIG. 10, Panels D and E, which demonstrate that treatment with the compound resulted in degradation of both PAX3::FOXO1 and was not due to decreased protein synthesis. This is also reflected in the half-life measurement of PAX3::FOXO1 in CHX and Compound 07-A-treated cells, which was 6.324 and 1.326 hours, respectively. These results demonstrate that when new protein synthesis was blocked and PAX3::FOXO1 decay was observed, Compound 07-A accelerates PAX3::FOXO1 degradation and reduces its half-life.

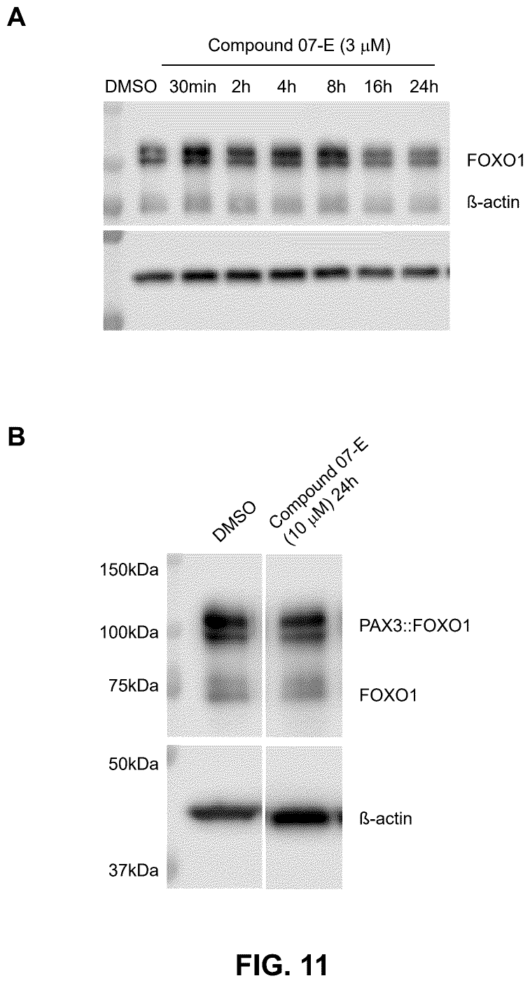

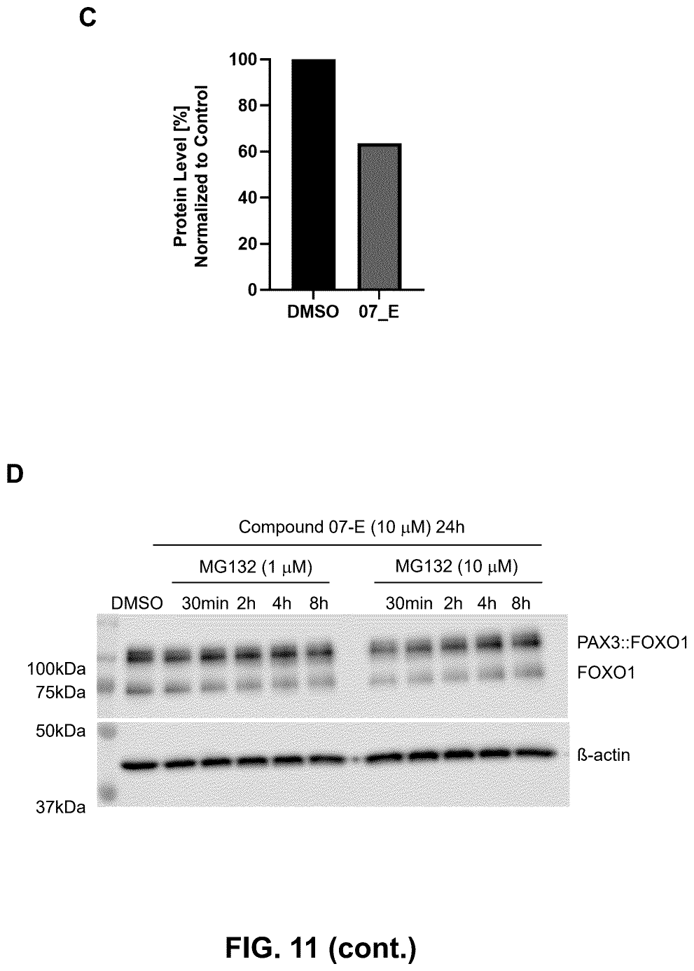

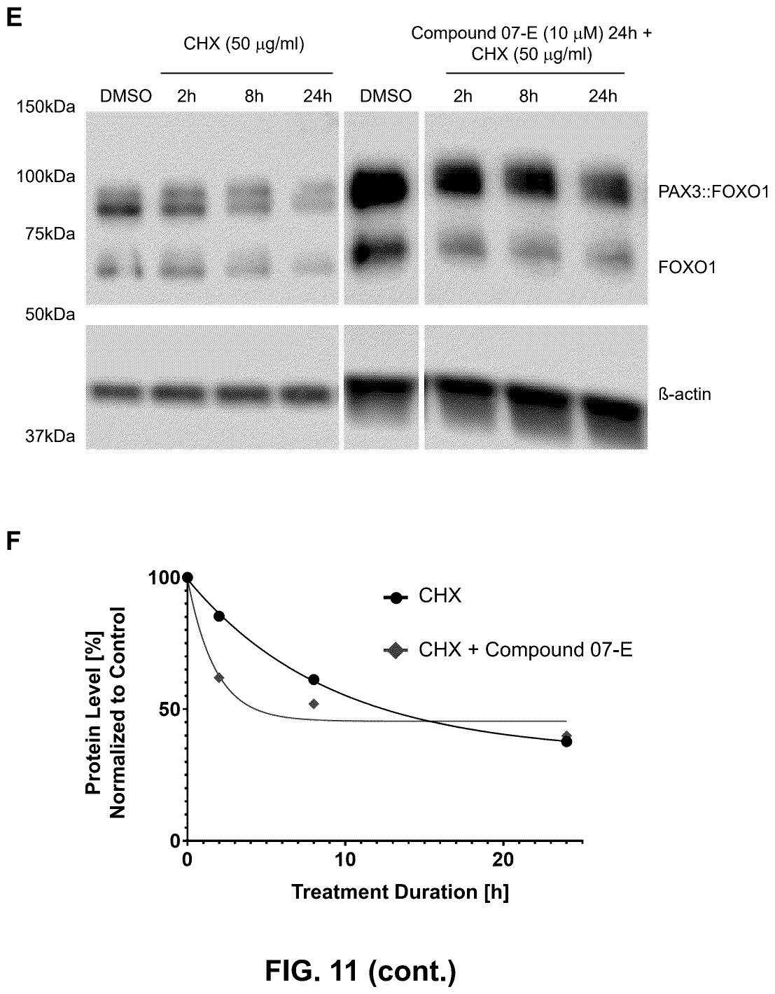

Compound 07-E was analyzed using three assays. In the first assay, RH30 cells were exposed to the PROTAC compound for up to 24 hours at concentration of 3 μM, and for 24 hours at a concentration of 10 μM. The results of the Western blot analysis are shown in FIG. 11, Panels A and B, and indicate that the degradation of PAX3::FOXO1 increases over time. Also, quantification of the PAX3::FOXO1 fusion protein in the cells show that protein level is about 40% less in cells treated by Compound 07-E as compared to DMSO control (see FIG. 11, Panel C). In the second assay, RH30 cells were treated with the PROTAC compound for 24 hours at a concentration of 10 μM and with MG132 at a concentration of 1 or 10 μM for 2, 4, or 8 hours. The results are shown in FIG. 11, Panel D, and confirm that, when treated with Compound 07-E, degradation of PAX3::FOXO1 is through the proteosome in the cell. The third assay was a CHX chase assay, in which RH30 cells were pre-treated with 50 μg/ml of CHX for 2, 8, or 24 hours, prior to treating with Compound 07-E at concentration of 10 μM. Results are shown in FIG. 11, Panels E and F, which demonstrate that treatment with the compound resulted in degradation of both PAX3::FOXO1 and was not due to decreased protein synthesis. This is also reflected in the half-life measurement of PAX3::FOXO1 in CHX- and Compound 07-E-treaterd cells, which was 6.324 and 1.209 hours, respectively. These results show that when new protein synthesis was blocked and PAX3::FOXO1 decay was observed, Compound 07-E accelerates PAX3::FOXO1 degradation and reduces its half-life.

### Example 5

A study was conducted in which three PROTAC compounds, comprising C6.12 or C6.15 or C6.18 as the PAX3::FOXO1 fusion protein binding moiety and AD-5-47a as the E3 ubiquitin ligase binding moiety, were evaluated for their cytotoxicity in RH30 cells. The PROTAC compounds are Compounds G-3A, G-3B, and G-3C described in the Compounds of the Present Invention section, infra.

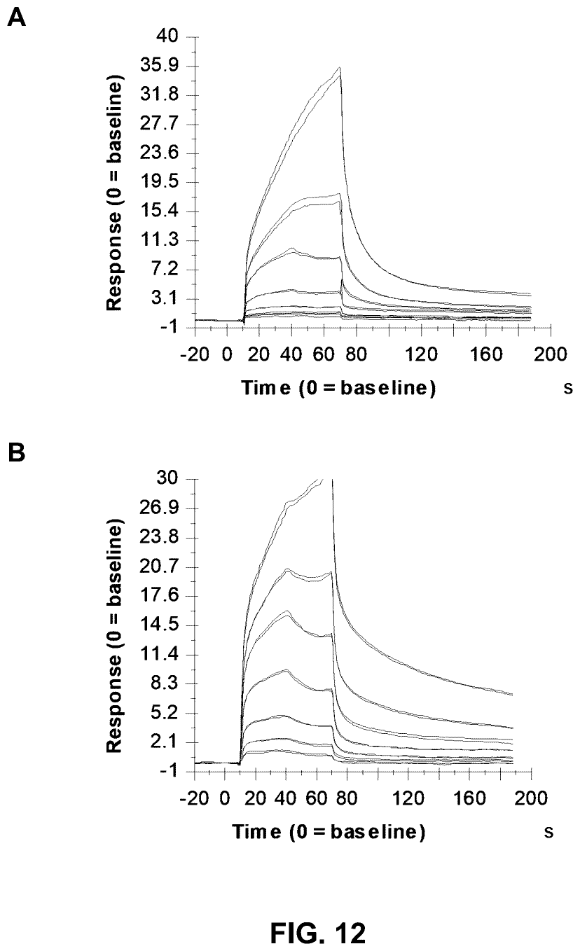

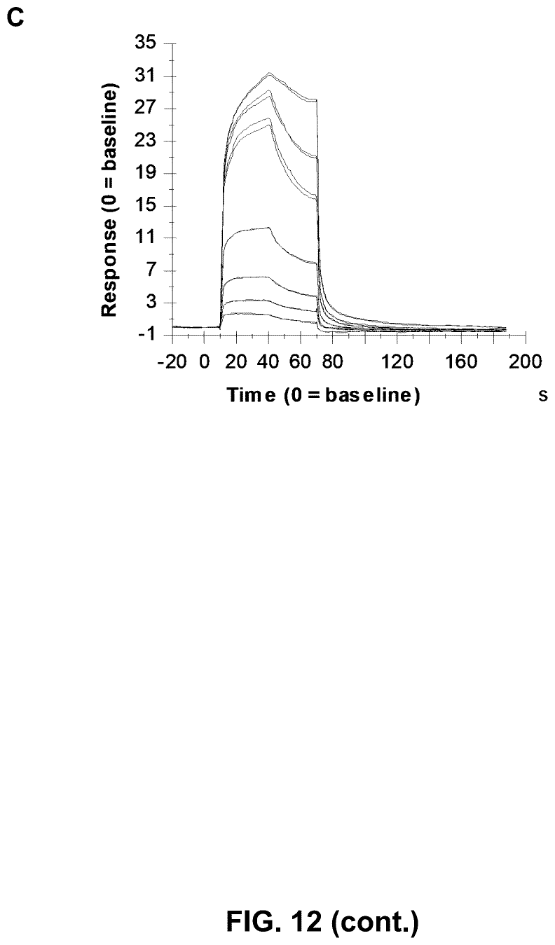

To calculate the binding affinity of the compounds to PAX3::FOXO1 protein, SPR experiments were conducted in which the bound PAX3::FOXO1 protein was exposed to the compounds at concentrations of 1.25, 2.5, 5, 10, 20, 40, and 80 μM. A binding affinity (K_{D}) of 38.3 μM, 27.2 μM, and 29.3 μM was detected between the PAX3::FOXO1 protein and Compounds G-3A, G-3B, and G-3C, respectively. Binding sensorgrams are presented in FIG. 12, Panels A-C.

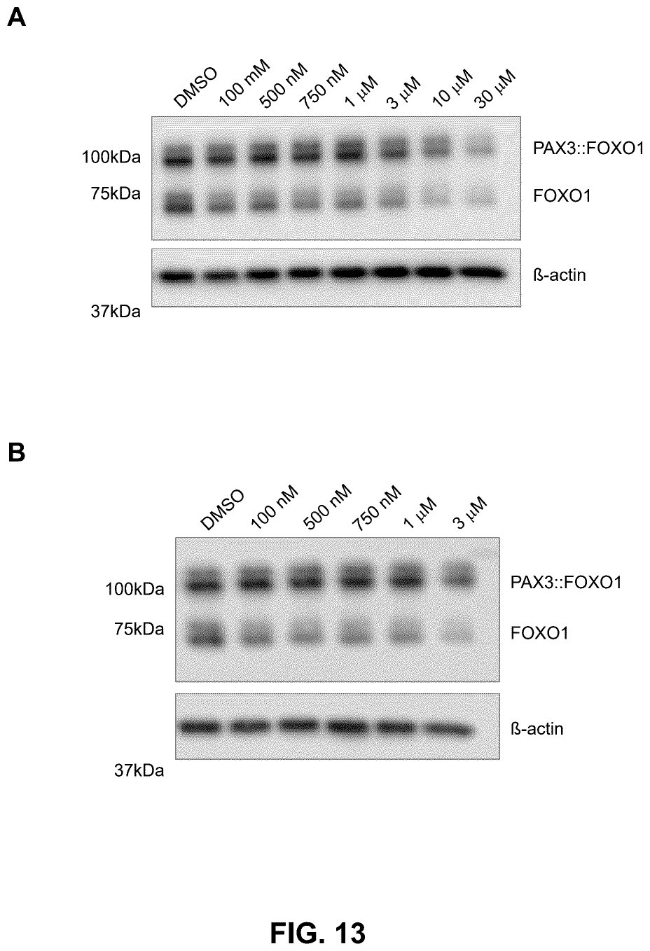

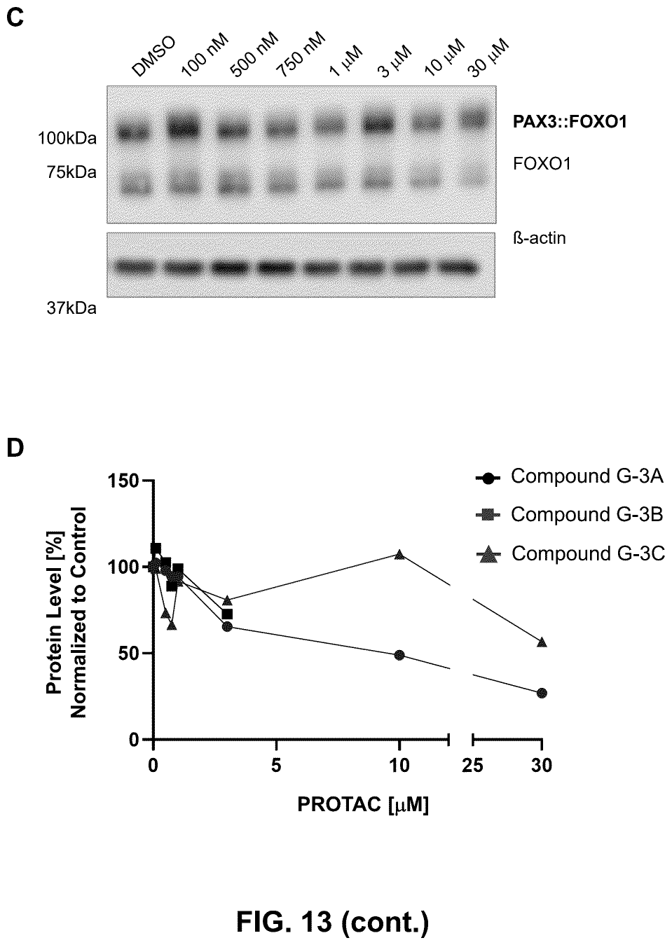

In a second assay, RH30 cells were exposed to the PROTAC compounds for 24 hours at concentrations of 100 nM, 500 nM, 750 nM, 1 μM, 3 μM, 10 μM, and 30 μM. The results of the Western blot analysis are shown in FIG. 13, Panels A-C, which show degradation of PAX3::FOXO1 that increases as the concentration of the compound increases. Quantification of the PAX3::FOXO1 fusion protein in the cells show a similar effect of increasing the concentration of the compound (see FIG. 13, Panel D).

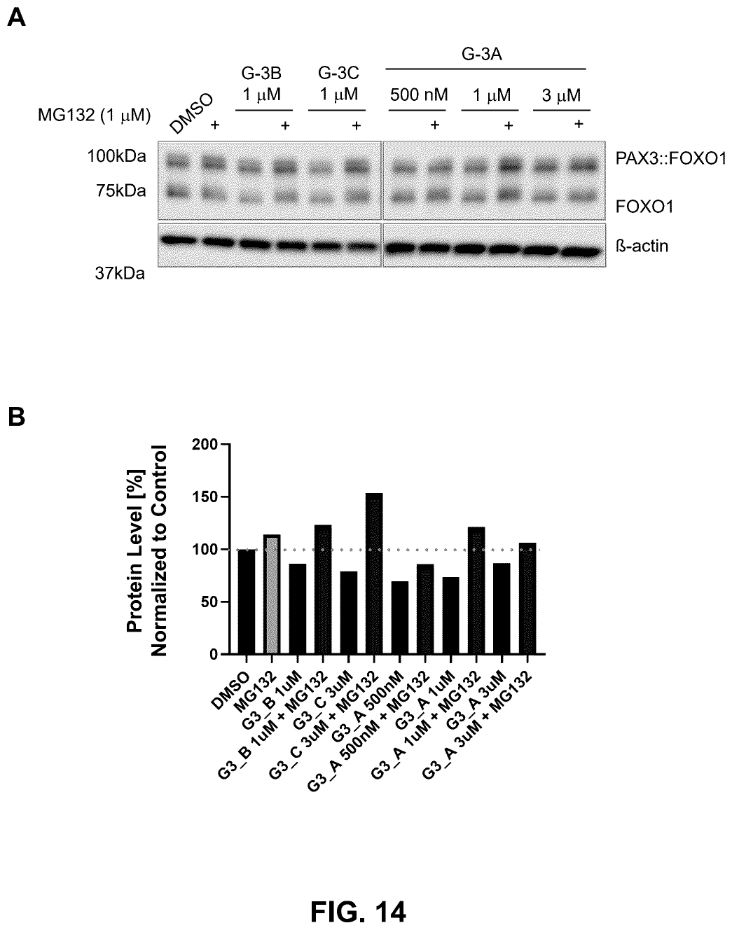

In a third assay, RH30 cells were treated with the PROTAC compounds for 24 hours at concentrations of 500 nM (for Compound G-3A only), 1 μM, and 3 μM, and with MG132 at a concentration of 1 μM. The results are shown in FIG. 14, Panels A and B, and confirm that degradation of PAX3::FOXO1 is through the proteosome in the cell.

### REFERENCES

[1] Shern J F, et al., Pediatric Rhabdomyosarcoma, Critical Reviews in Oncogenesis, 2015, 20(3-4): 227-243.
[2] Skapek S X, et al., Rhabdomyosarcoma, Nature Reviews: Disease Primers, 2020, 5(1): 1.
[3] Raze T, et al., PAX-FOXO1 fusion status in children and adolescents with alveolar rhabdomyosarcoma: impact on clinical, pathological, and survival features, Pediatric Blood & Cancer, 2023, 70(4): e30228.
[4] Sorensen P H, et al., PAX3-FKHR and PAX7-FKHR gene fusions are prognostic indicators in alveolar rhabdomyosarcoma: a report from the children's oncology group, Journal of Clinical Oncology, 2002, 20(11): 2672-2679.
[5] McEvoy M T, et al., Pediatric rhabdomyosarcoma incidence and survival in the United States: An assessment of 5656 cases, 2001-2017, Cancer Medicine, 2023, 12(3): 3644-3656.
[6] Barr F G, et al., Rearrangement of the PAX3 paired box gene in the paediatric solid tumour alveolar rhabdomyosarcoma, Nature Genetics, 1993, 3(2): 113-117.
[7] Davis R J, et al., Fusion of PAX7 to FKHR by the variant t(1; 13)(p36; q14) translocation in alveolar rhabdomyosarcoma, Cancer Research, 1994, 54(11): 2869-72.
[8] Barr F G, Gene fusions involving PAX and FOX family members in alveolar rhabdomyosarcoma, Oncogene, 2001, 20(40): 5736-5746.
[9] Skapek S X, et al., PAX-FOXO1 fusion status drives unfavorable outcome for children with rhabdomyosarcoma: a Children's Oncology Group report, Pediatric Blood & Cancer, 2013, 60(9): 1411-1417.
Fredericks W J, et al., The PAX3-FKHR fusion protein created by the t(2; 13) translocation in alveolar rhabdomyosarcomas is a more potent transcriptional activator than PAX3, Molecular and Cellular Biology, 1995, 15(3): 1522-1535.
Bennicelli J L, et al., Mechanism for transcriptional gain of function resulting from chromosomal translocation in alveolar rhabdomyosarcoma, Proceedings of the National Academy of Sciences USA, 1996, 93(11): 5455-5459.
Bennicelli J L, et al., PAX3 and PAX7 exhibit conserved cis-acting transcription repression domains and utilize a common gain of function mechanism in alveolar rhabdomyosarcoma, Oncogene, 1999, 18(3): 4348-4356.
Pandey P R, et al., PAX3-FOXO1 is essential for tumour initiation and maintenance but not recurrence in a human myoblast model of rhabdomyosarcoma, Journal of Pathology, 2017, 241(5): 626637.
Kikuchi K, et al., Cell-cycle dependent expression of a translocation-mediated fusion oncogene mediates checkpoint adaptation in rhabdomyosarcoma, PLOS Genetics, 2014, 10(1): e1004107.
Jothi M, et al., Small molecule inhibition of PAX3-FOXO1 through AKT activation suppresses malignant phenotypes of alveolar rhabdomyosarcoma, Moleclar Cancer Therapeutics, 2013, 12(12): 2663-2674.
Abraham J, et al., Lineage of origin in rhabdomyosarcoma informs pharmacological response, Genes & Development, 2014, 28(14): 1578-1591.
Herrero M D, et al., Cell-based small-molecule compound screen identifies fenretinide as potential therapeutic for translocation-positive rhabdomyosarcoma, PLOS One, 2013, 8(1): e55072.
Gryder B E, et al., PAX3-FOXO1 establishes myogenic super enhancers and confers BET bromodomain vulnerability, Cancer Discovery, 2017, 7(8): 884-889.
Ghayad S E, et al., The histone deacetylase inhibitor Suberoylanilide Hydroxamic Acid (SAHA) as a therapeutic agent in rhabdomyosarcoma, Cancer Biology & Therapy, 2019, 20(3): 272-283.
Bharathy N, et al., Preclinical testing of the glycogen synthase kinase-3β inhibitor tideglusib for rhabdomyosarcoma, Oncotarget, 2017, 8(38): 62976-62983.
Kaylani S Z, et al., Rapamycin targeting mTOR and hedgehog signaling pathways blocks human rhabdomyosarcoma growth in xenograft murine model, Biochemical and Biophysical Research Communications, 2013, 435(4): 557-561.
Li S Q, et al., Targeting wild-type and mutationally activated FGFR4 in rhabdomyosarcoma with the inhibitor ponatinib (AP24534), PLOS One, 2013, 8(10): e76551.
Ishita T & Ciulli A, E3 ligase ligands for PROTACs: how they were found and how to discover new ones, SLAS Discovery, 2021, 26(4): 484-502.
Olaru A, et al., Surface plasmon resonance (SPR) biosensors in pharmaceutical analysis, Critical Reviews in Analytical Chemistry, 2015, 45(2): 97-105.
Velazquez-Campoy A, et al., Characterization of protein-protein interactions by isothermal titration calorimetry, Methods in Molecular Biology, 2015, 1278:183-204.
Wienken C J, et al., Protein-binding assays in biological liquids using microscale thermophoresis, Nature Communications, 2010, 1:100.
Sultana A & Lee J E, Measuring protein-protein and protein-nucleic Acid interactions by biolayer interferometry, Current Protocols in Protein Science, 2015, 79:19.25.1-19.25.26.
Niesen F H, et al., The use of differential scanning fluorimetry to detect ligand interactions that promote protein stability, Nature Protcols, 2007, 2(9): 2212-2221.
Lea W A & Simeonov A, Fluorescence polarization assays in small molecule screening, Expert Opinion on Drug Discovery, 2011, 6(1): 17-32.
Cala O, et al., NMR-based analysis of protein-ligand interactions, Analytucal and Bioanalytical Chemistry, 2014, 406(4): 943-956.
Hong S H, et al., Exploiting the cullin E3 ligase adaptor protein SKP1 for targeted protein degradation, ACS Chemical Biology, 2024, 19(2): 442-450.
Eisenhauer E A, et al., New response evaluation criteria in solid tumours: Revised RECIST guideline (version 1.1), European Journal of Cancer, 2009, 45(2): 228-247.
]Nakazawa K, et al., Piperacetazine directly binds to the PAX3::FOXO1 fusion protein and inhibits its transcriptional activity, Cancer Research Communications, 3(10): 2030-2043.

1. A compound of formula (I): 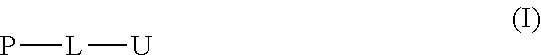
or a pharmaceutically acceptable salt thereof, wherein
P is a PAX3::FOXO1 fusion protein binding moiety;
L is a bivalent linker; and
U is an E3 ubiquitin ligase binding moiety; and
wherein the PAX3::FOXO1 fusion protein binding moiety comprises an agent selected from Table 1 or a derivative thereof.

2. The compound of claim 1, wherein the PAX3::FOXO1 fusion protein binding moiety comprises an agent selected from NSC697468, alcuronium chloride, NSC2805, didanosine, carboplatin, piperacetazine, and derivatives thereof.

3. The compound of claim 1, wherein the PAX3::FOXO1 fusion protein binding moiety comprises a derivative of piperacetazine selected from C6.12, C6.16, C6.21, C6.14, C6.15, C6.18a, and C.6.18b, or a pharmaceutically acceptable salt thereof.

4. (canceled)

5. The compound of claim 1, wherein the ubiquitin ligase binding moiety comprises a ligand that binds to an E3 ubiquitin ligase selected from Von Hippel-Lindau tumor suppressor protein (VHL), cereblon (CRBN), cellular inhibitor of apoptosis protein (cIAP), murine double minute 2 (MDM2), and S-phase kinase-associated protein 1 (SKP-1).

6. The compound of claim 5, wherein the E3 ubiquitin ligase binding moiety comprises a ligand that binds to VHL, wherein the ligand is selected from VH032, VH101, VH298, and derivatives thereof.

7. (canceled)

8. The compound of claim 5, wherein the E3 ubiquitin ligase binding moiety comprises a ligand that binds to CRBN, wherein the ligand is selected from thalidomide, pomalidomide, lenalidomide, and derivatives thereof.

9-13. (canceled)

14. The compound of claim 5, wherein the E3 ubiquitin ligase binding moiety comprises a ligand that binds to SKP-1, wherein the ligand is EN884, or a derivative thereof selected from AD-5-49, AD-5-47a, and AD-5-47b.

15-16. (canceled)

17. The compound of claim 1, wherein the bivalent linker comprises one or more alkyl chains, polyethylene glycol (PEG) chains, extended glycol chains, or a combination thereof.

18-19. (canceled)

20. The compound of claim 8, wherein the E3 ubiquitin ligase binding moiety comprises pomalidomide.

21. The compound of claim 20, wherein the PAX3::FOXO1 fusion protein binding moiety comprises NSC697468, and the compound is selected from the following:

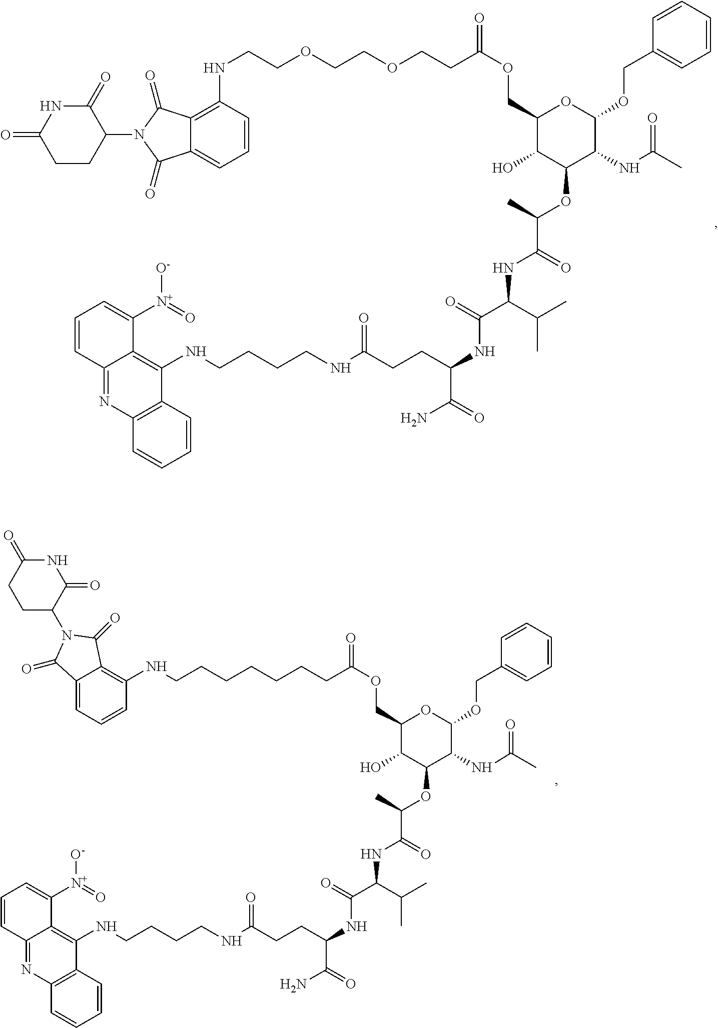 [SMILES: CC(=O)N[C@H]1[C@@H](OCc2ccccc2)O[C@H](COC(=O)CCOCCOCCNc2cccc3c2C(=O)N(C2CCC(=O)NC2=O)C3=O)[C@@H](O)[C@@H]1O[C@H](C)C(=O)N[C@H](C(=O)N[C@H](CCC(=O)NCCCCNc1c2ccccc2nc2cccc([N+](=O)[O-])c12)C(N)=O)C(C)C ; CC(=O)N[C@H]1[C@@H](OCc2ccccc2)O[C@H](COC(=O)CCCCCCCNc2cccc3c2C(=O)N(C2CCC(=O)NC2=O)C3=O)[C@@H](O)[C@@H]1O[C@H](C)C(=O)N[C@H](C(=O)N[C@H](CCC(=O)NCCCCNc1c2ccccc2nc2cccc([N+](=O)[O-])c12)C(N)=O)C(C)C]

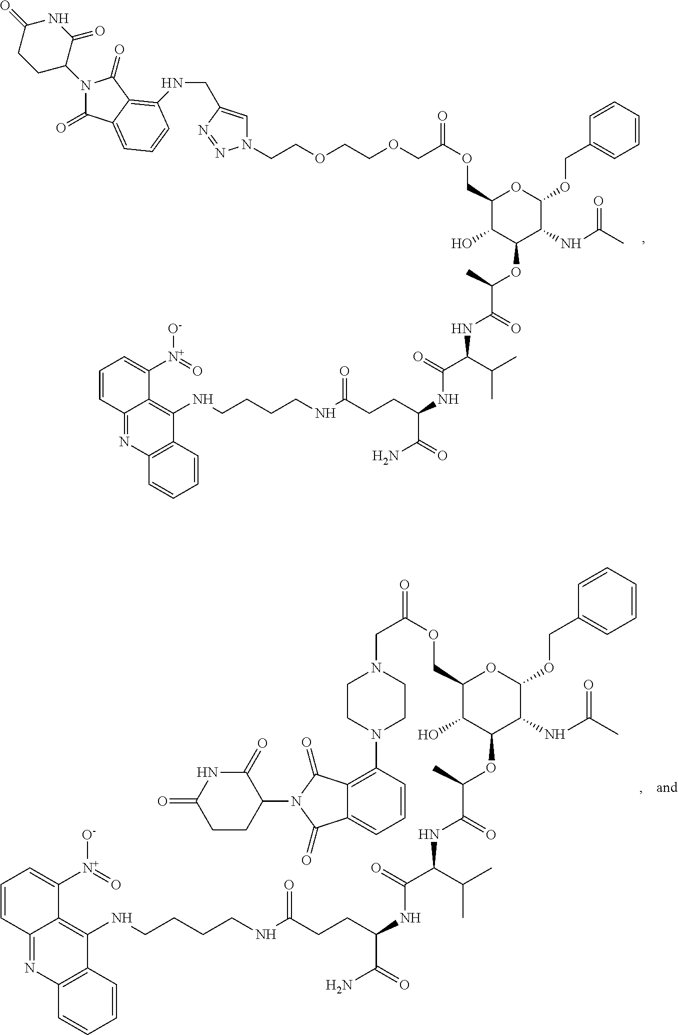 [SMILES: CC(=O)N[C@H]1[C@@H](OCc2ccccc2)O[C@H](COC(=O)COCCOCCn2cc(CNc3cccc4c3C(=O)N(C3CCC(=O)NC3=O)C4=O)nn2)[C@@H](O)[C@@H]1O[C@H](C)C(=O)N[C@H](C(=O)N[C@H](CCC(=O)NCCCCNc1c2ccccc2nc2cccc([N+](=O)[O-])c12)C(N)=O)C(C)C ; CC(=O)N[C@H]1[C@@H](OCc2ccccc2)O[C@H](COC(=O)CN2CCN(c3cccc4c3C(=O)N(C3CCC(=O)NC3=O)C4=O)CC2)[C@@H](O)[C@@H]1O[C@H](C)C(=O)N[C@H](C(=O)N[C@H](CCC(=O)NCCCCNc1c2ccccc2nc2cccc([N+](=O)[O-])c12)C(N)=O)C(C)C]

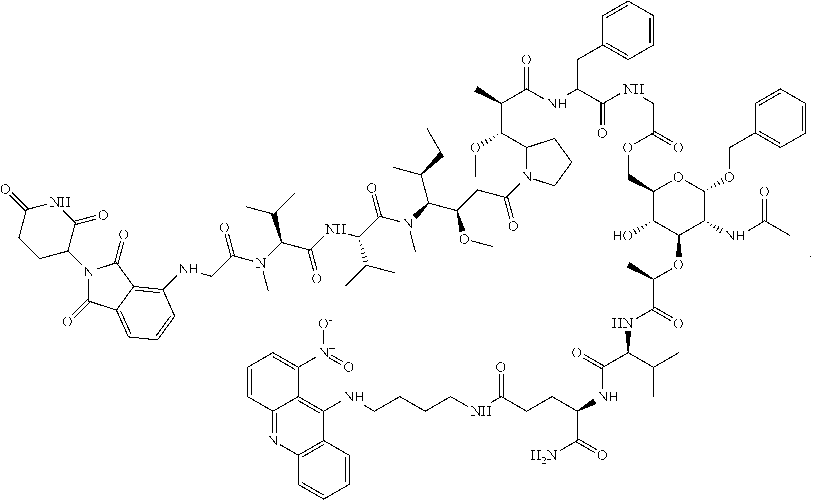 [SMILES: CC[C@H](C)[C@@H]([C@@H](CC(=O)N1CCCC1[C@H](OC)[C@@H](C)C(=O)NC(Cc1ccccc1)C(=O)NCC(=O)OC[C@H]1O[C@H](OCc2ccccc2)[C@H](NC(C)=O)[C@@H](O[C@H](C)C(=O)N[C@H](C(=O)N[C@H](CCC(=O)NCCCCNc2c3ccccc3nc3cccc([N+](=O)[O-])c23)C(N)=O)C(C)C)[C@@H]1O)OC)N(C)C(=O)[C@@H](NC(=O)[C@H](C(C)C)N(C)C(=O)CNc1cccc2c1C(=O)N(C1CCC(=O)NC1=O)C2=O)C(C)C]

22. (canceled)

23. The compound of claim 20, wherein the PAX3::FOXO1 fusion protein binding moiety is selected from C6.12, C6.16, C6.21, C6.14, C6.15, and a pharmaceutically acceptable salt thereof, and the compound is selected from the following:

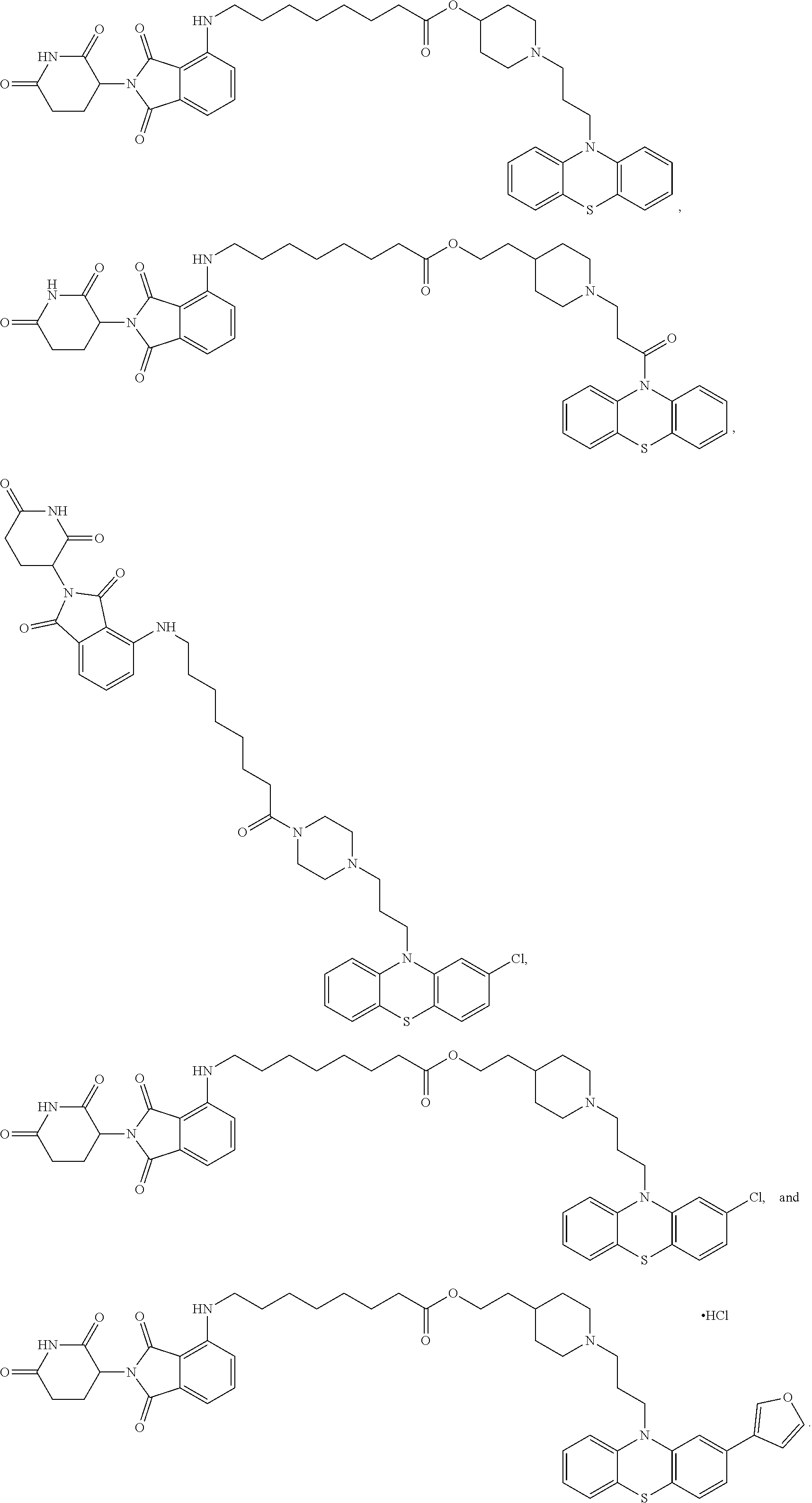

24. (canceled)

25. The compound of claim 20, wherein the PAX3::FOXO1 fusion protein binding moiety comprises C6.18b or a pharmaceutically acceptable salt thereof, and the compound is selected from the following:

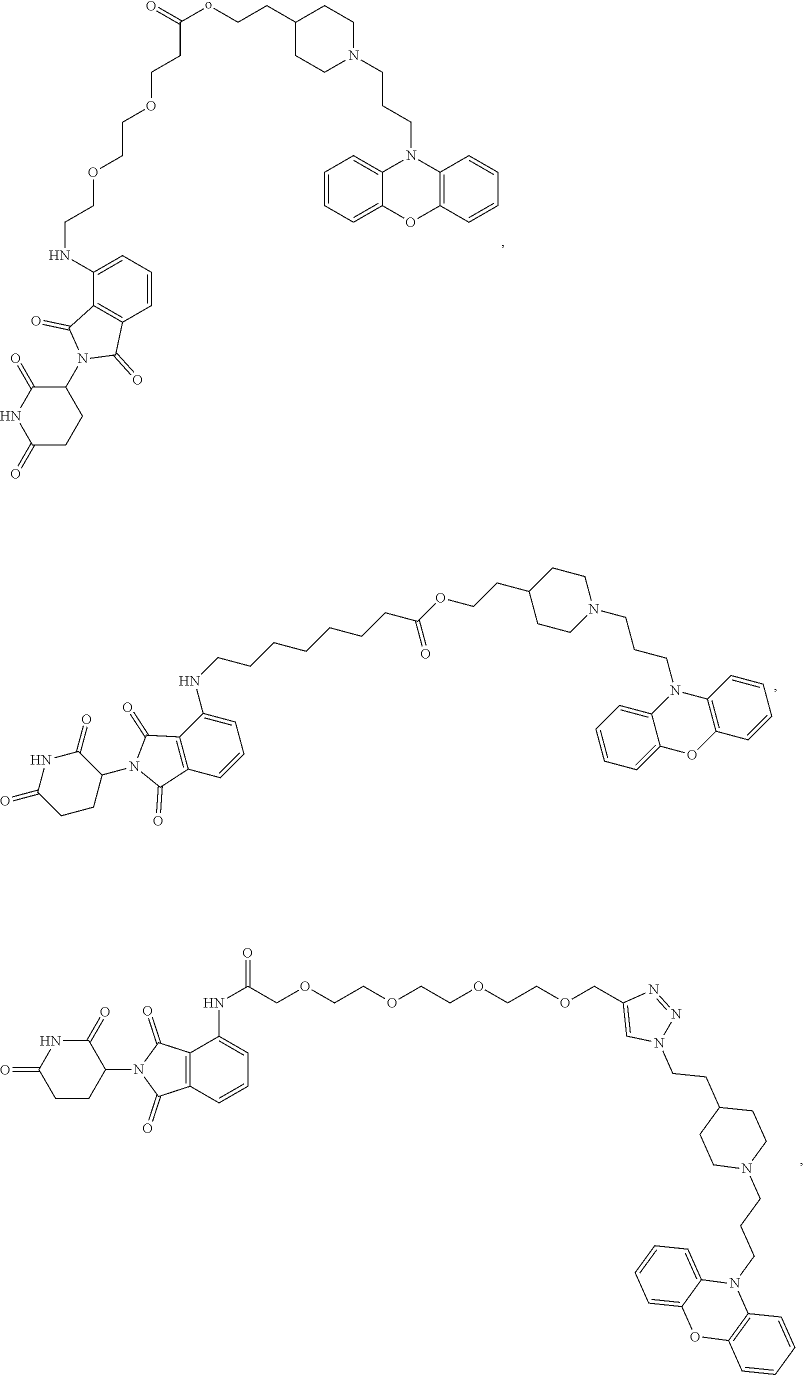

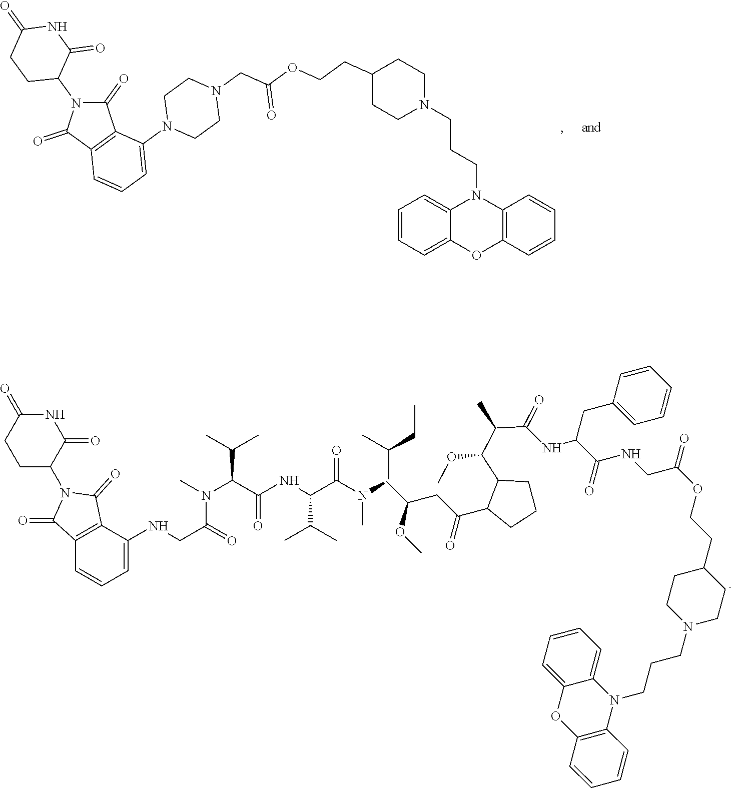

26. The compound of claim 14, wherein the E3 ubiquitin ligase binding moiety comprises AD-5-47a.

27. The compound of claim 26, wherein the PAX3::FOXO1 fusion protein binding moiety is selected from C6.12, C6.15, C6.18b, and a pharmaceutically acceptable salt thereof, and the compound is selected from the following:

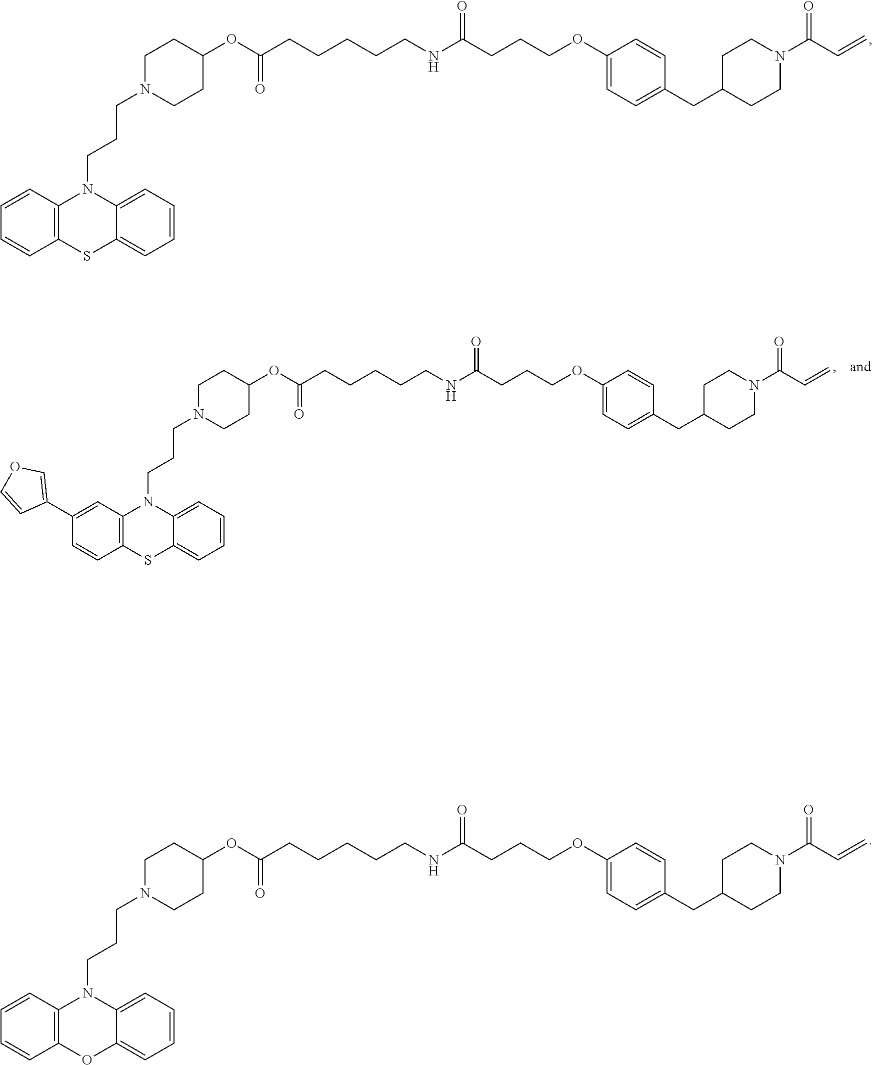 [SMILES: C=CC(=O)N1CCC(Cc2ccc(OCCCC(=O)NCCCCCC(=O)OC3CCN(CCCN4c5ccccc5Sc5ccccc54)CC3)cc2)CC1 ; C=CC(=O)N1CCC(Cc2ccc(OCCCC(=O)NCCCCCC(=O)OC3CCN(CCCN4c5ccccc5Sc5ccc(-c6ccoc6)cc54)CC3)cc2)CC1 ; C=CC(=O)N1CCC(Cc2ccc(OCCCC(=O)NCCCCCC(=O)OC3CCN(CCCN4c5ccccc5Oc5ccccc54)CC3)cc2)CC1]

28. A pharmaceutical composition comprising a compound of claim 1, and a pharmaceutically acceptable carrier.

29. A method of reducing PAX3::FOXO1 protein levels in a subject with fusion-positive rhabdomyosarcoma, the method comprising administering the pharmaceutical composition of claim 28 to the subject.

30. The method of claim 29, wherein the subject has fusion-positive alveolar rhabdomyosarcoma.

31. A method of ubiquitinating a PAX3::FOXO1 fusion protein in a cell, the method comprising administering the pharmaceutical composition of claim 28 to the cell.

32-33. (canceled)

34. A method of treating fusion-positive rhabdomyosarcoma in a subject in need thereof, the method comprising administering the pharmaceutical composition of claim 28 to the subject.

35. (canceled)

36. A method of treating a disease caused by the overexpression of PAX3::FOXO1 protein in a subject in need thereof, the method comprising administering the pharmaceutical composition of claim 28 to the subject.

37. (canceled)


SEQUENCE LISTING (4 sequences)

SEQ ID NO: 1 - DNA, 25 bp, synthetic construct
  source 1..25: mol_type = other DNA
accgtgactaatttaattagtcacg

SEQ ID NO: 2 - DNA, 25 bp, synthetic construct
  source 1..25: mol_type = other DNA
cgtgactaattaaattagtcacggt

SEQ ID NO: 3 - DNA, 34 bp, synthetic construct
  source 1..34: mol_type = other DNA
accggaaggaaggaaggaaggaaggaaggaagtg

SEQ ID NO: 4 - DNA, 34 bp, synthetic construct
  source 1..34: mol_type = other DNA
tggccttccttccttccttccttccttccttcac